(19) United States
(12) Patent Application Publication
(10) Pub. No.: US 2018/0209956 A1
(43) Pub. Date: Jul. 26, 2018

(54) HTS Assay for Identifying Small Molecule Inhibitors of RAD52 and Uses of Identified Small Molecule Inhibitors for Treatment and Prevention of BRCA-Deficient Malignancies

(71) Applicant: University of Iowa Research Foundation, Iowa City, IA (US)

(72) Inventors: Michael Ashley Spies, Iowa City, IA (US); Maria Spies, Iowa City, IA (US); Sarah R. Hengel, Coralville, IA (US)

(73) Assignee: University of Iowa Research Foundation, Iowa City, IA (US)

(21) Appl. No.: 15/745,587

(22) PCT Filed: Jul. 11, 2016
(86) PCT No.: PCT/US2016/042730

§ 371 Date: Jan. 17, 2018

Related U.S. Application Data
(60) Provisional application No. 62/347,216, filed on Jun. 8, 2016.
(60) Provisional application No. 62/193,908, filed on Jul. 17, 2015.

(51) Int. Cl. G01N 33/50; A61K 31/05; A61P 35/00; A61K 31/65; A61K 31/353; A61K 31/4468; A61K 31/122; A61K 31/341; A61K 31/19; C40B 30/02

(52) U.S. Cl. CPC G01N 33/502; A61K 31/05; A61P 35/00; A61K 31/65; C40B 30/02; A61K 31/4468; A61K 31/122; A61K 31/341; A61K 31/19; A61K 31/353

(57) ABSTRACT

Disclosed are methods, compositions, kits, and systems for identifying small-molecule drugs for treating cancer in a subject. The disclosed methods, compositions, kits, and systems may be utilized to identify small-molecule inhibitors of radiation sensitive protein 52 (RAD52) in order to treat cancer in a subject, such as breast cancer in a subject having a BRCA1-deficient, BRCA2-deficient, and/or PALB2-deficient phenotype by administering the identified small-molecule inhibitors to the subject.

### CROSS-REFERENCE TO RELATED PATENT APPLICATIONS

The present application claims the benefit of priority under 35 U.S.C. § 119(e) to U.S. Provisional Application No. 62/347,216, filed on Jun. 8, 2016, and to U.S. Provisional Application No. 62/193908, on Jul. 17, 2015, the contents of which are incorporated herein by reference in their entireties.

### STATEMENT REGARDING FEDERALLY SPONSORED RESEARCH OR DEVELOPMENT

This invention was made with government support under R01-GM097373 awarded by the National Institutes of Health. The government has certain rights in the invention.

### BACKGROUND

The field of the invention relates to methods, compositions, kits, and systems for identifying small-molecule drugs for treating cancer in a subject. In particular, the field of the invention relates to methods, compositions, kits, and systems for identifying small-molecule inhibitors of radiation sensitive protein 52 (RAD52) for treating cancer in a subject, such as breast cancer in a subject having a BRCA1-deficient, BRCA2-deficient, and/or PALB2-deficient phenotype.

### SUMMARY

Disclosed are methods, compositions, kits, and systems for identifying small-molecule drugs for treating cancer in a subject. The disclosed methods, compositions, kits, and systems may be utilized to identify small-molecule inhibitors of radiation sensitive protein 52 (RAD52) in order to treat cancer in a subject, such as breast cancer in a subject having a BRCA1-deficient, BRCA2-deficient, and/or PALB2-deficient phenotype.

The disclosed method may include screening methods for identifying inhibitors of RAD52 biological activity. The screening methods may include contacting RAD52 with a compound and determining if the compound binds RAD52 and inhibits binding of RAD52 to ssDNA.

Compounds identified by the screening methods may be formulated as pharmaceutical compositions for treating cancers associated with RAD52 biological activity. The disclosed methods of treating may include administering the pharmaceutical compositions to a subject in need thereof in order to treat and/or prevent cancer or a cell proliferative disorder in the subject, such as breast cancer, and in particular BRCA1-deficient, BRCA2-deficient, and/or PALB2-deficient cancer.

Also disclosed are small-molecule inhibitors identified by the disclosed methods for treating cancers associated with RAD52 biological activity, such as breast cancer in a subject having a BRCA1-deficient, BRCA2-deficient, and/or PALB2-deficient phenotype. The identified small-molecule inhibitors may be formulated as a pharmaceutical composition for treating cancers associated with RAD52 biological activity.

### BRIEF DESCRIPTION OF THE FIGURES

FIG. 1: High Throughput Screening of the MicroSource SPECTRUM collection identifies 12 compounds that inhibit the RAD52-ssDNA interaction. a) Control lanes from a 384 well: 16 negative control wells contain stoichiometric RAD52-Cy3-dT₃₀-Cy5 complexes (top filled circles), while 16 positive control wells contain a stoichiometric RAD52-Cy3-dT₃₀-Cy5 complex challenged with unlabeled polydT100 (bottom filled circles). Top and bottom lines with error bars at the ends indicate the average and the standard deviation for the negative and positive controls, respectively. Z′ factor of 0.94 was calculated for these control lanes, indicating excellent reliability of the assay. b) A representative 384 well plate from the HTS screen highlighting ‘1’, ‘5’, ‘6’, ‘7’, and ‘15’. Top and bottom lines with error bars at the ends indicate the average and the standard deviation for the negative and positive controls, respectively. c) Average of cherry-picked rescreening of compounds identified from screening all plates in the MicroSource SPECTRUM collection highlighting all 12 identified hits (numbered filled circles) along with a number of false positive compounds (non-numbered filled circles) that either showed poor reproducibility in subsequent rescreening or a linear dependence of the signal on the compound concentration. Top and middle lines with error bars at the ends indicate the average and the standard deviation for the negative and positive controls, respectively.

FIG. 2: Biochemical characterization of ‘1’. a) Aromatic region of the 1D ¹H NMR spectrum of compound ‘1’ alone (top line) and the WaterLOGSY spectrum of 20 μM compound ‘1’ in the presence of 3.3 μM RAD52 (bottom line). The nonexchangeable proton peaks are labeled using atom names as indicated on the structure of compound ‘1’. b) IC₅₀ values for inhibition of ssDNA binding and wrapping were determined using FRET-based assays that follow the change in geometry of a Cy3-dT30-Cy5 substrate (black circles). Computed IC₅₀ value is shown below the curve. Titration of the RAD52-dsDNA with ‘1’ (grey boxes) shows no perturbation of the dsDNA binding. c) IC₅₀ values for inhibition of RAD52-mediated ssDNA annealing were determined by fitting the dependence of the extent of oligonucleotide-based annealing reaction carried in the presence of increasing concentration of ‘1’. d) Aromatic region of the 1D ¹H NMR spectrum of compound ‘1’ alone (top line) and the WaterLOGSY spectrum of 20 μM compound ‘1’ in the presence of 3.3 μM RPA (bottom). e) Titration of the RAD52-RPA-Cy3-dT₃₀-Cy5 complex with ‘1’ (black circles). Computed IC₅₀ value is shown below the curve. Green squares show titration of the RPA-Cy3-dT₃₀-Cy5 complex with ‘1’. f) IC₅₀ values for inhibition of RAD52-mediated annealing of the RPA-coated ssDNA were determined by fitting the dependence of the extent of the annealing reaction carried out in the presence of increasing concentration of ‘1’.

FIG. 3: Stoichiometric complexes of RAD52 with ssDNA, RPA-coated ssDNA and dsDNA yield characteristic FRET values. a) FRET measurements were performed by titrating RAD52 protein into a solution containing 1 nM Cy3-dT₃₀-Cy5 ssDNA. The Cy3 fluorescence was excited directly and the emissions of Cy3 and Cy5 dyes were measured, and the respective FRET values calculated as described in the Materials and Methods. The highest separation in the FRET signal of unbound ssDNA and RAD52-ssDNA complex was achieved around 8 nM RAD52. At this concentration one RAD52 heptameric ring binds and wraps the ssDNA oligonucleotide bringing the Cy3 and Cy5 dyes close to one another. The details and control experiments for these measurements can be found in (Grimme et al., 2010, Grimme and Spies, 2011). Each data point represents average and standard deviation for at least three independent titrations. b) The titrations were performed similarly to those shown in a, except 1 nM RPA was added to a solution containing 1 nM Cy3-dT₃₀-Cy5 ssDNA prior to titration of RAD52. Under our experimental conditions, RPA forms stoichiometric complexes with the 30-mer DNA oligonucleotide with 1 RPA coating 1 molecule of ssDNA. The data points and error bars represent averages and standard deviation for three or more independent titrations. c) The titrations were performed similarly to those shown in a, except 1 nM dsDNA (Cy3-Oligo28-Cy5 annealed to Oligo28-REV) was used as a substrate. Stoichiometric complexes are achieved at 10 nM RAD52. The data points and error bars represent averages and standard deviation for three or more independent titrations.

FIG. 4: RAD52 FRET based ssDNA annealing assay in the presence of small molecules. a) Schematic of the FRET based annealing reaction. In two half-reactions, stoichiometric amounts of RAD52 were incubated with Target28Cy3 and Probe28Cy5 oligonucleotides, respectively. Upon mixing of the two half-reactions, RAD52 facilitated annealing of the two complementary oligonucleotides, which can be observed as an increase in FRET between Cy3 and Cy5 dyes. b) Annealing reactions performed in the absence (top line) or presence of increasing concentrations of ‘1’. The average of three or more independent annealing reactions are shown for each curve. Grey continuous lines show fits to double exponentials. Bottom dots correspond to the DNA only reaction and the annealing reaction containing the DNA and 100 μM ‘1’.

FIG. 5: None of the tested compounds affect the oligomeric state of RAD52 protein. Possible effect of the identified compounds on the oligomeric state of RAD52 protein was probed in the dynamic light scattering experiments, which measured the average hydrodynamic radius of RAD52 (15.8 μM) alone or in the presence of equimolar concentrations of each compound. Measurements were recorded at 25° C. in buffer containing 50 mM Tris-HCl pH7.5, 200 mM KCl, 1 mM DTT, and 0.5 mM EDTA. A total of 10 measurements with 10 accumulations were collected, had monomodal distributions, and sum of squares (SOS) values of ≤0.65. Measurements were recorded with a DynaPro NanoStar (Wyatt Tech. Corp.) and the hydrodynamic radius were calculated with the DYNAMICS software.

FIG. 6: Compounds ‘1’ and ‘6’ have no effect on the interaction between RAD52 and RPA proteins. Ni-NTA Agarose (15 uL buffer equilibrated bead slurry) was incubated with 3 μM RAD52, 3 μM RPA in the presence or absence of 3 μM ‘1’ or ‘6’, in the binding buffer (30 mM Tris-Acetate pH7.5, 1 mM βME, 150 mM KCl, 30 mM Imidazole, 5% glycerol, and 0.2% Nonidet P40 substitute). After 30 minutes incubation on a neutator at 4° C. samples were spun down, and the aliquots of unbound (“free”) proteins from each reaction were saved. Then the beads were washed and the bound proteins were eluted with 20 uL elution buffer (the same as the binding buffer, but with 400 mM Imidazole) and saved for gel electrophoresis. Free proteins and proteins co-eluted from the beads (“bound”) were separated on the 12% SDS PAGE gel. Lane 1 is a loading control, which shows RAD52 and the three subunits of RPA (RPA70, RPA35, and RPA14). The proteins and the compounds present in each reaction are indicated in the table above the gel. The carton on the left of the gel schematically depicts the experiment: RAD52 protein binds to the Ni-NTA beads through the interaction with its 6× His tag; RPA is untagged and can be retained on the beads only through a specific interaction with RAD52 (Grimme et al., 2010). The experiment was repeated three times (a representative gel is shown) and no change in the ratio of RAD52 and RPA co-eluted from the beads in the presence and absence of ‘1’ or ‘6’ was detected.

FIG. 7: Biochemical characterization of ‘6’. a) Aromatic region of the 1D ¹H NMR spectrum of compound ‘6’ alone (top line) and the WaterLOGSY spectrum of 40 μM compound ‘6’ in the presence of 3.3 μM RAD52 (bottom line). The nonexchangeable proton peaks are labeled using atom names as indicated on the structure of compound ‘6’. b) IC₅₀ values for inhibition of ssDNA binding and wrapping were determined using FRET-based assays that follow the change in geometry of a Cy3-dT30-Cy5 substrate (black circles). Computed IC₅₀ value is shown above the curve. Titration of the RAD52-dsDNA with ‘6’ (grey boxes) shows that this inhibitor also perturbs the RAD52-dsDNA interaction. c) IC₅₀ values for inhibition of RAD52-mediated ssDNA annealing were determined by fitting the dependence of the extent of oligonucleotide-based annealing reaction carried in the presence of increasing concentration of ‘6’. d) Aromatic region of the 1D ¹H NMR spectrum of compound ‘6’ alone (top line) and the WaterLOGSY spectrum of 40 μM compound ‘6’ in the presence of 3.3 μM RPA (bottom line). e) Titration of the RAD52-RPA-Cy3-dT₃₀-Cy5 complex with ‘6’ (black circles). Computed IC₅₀ value is shown below the curve. Green squares show titration of the RPA-Cy3-dT₃₀-Cy5 complex with ‘6’. f) IC₅₀ values for inhibition of RAD52-mediated annealing of the RPA-coated ssDNA were determined by fitting the dependence of the extent of the annealing reaction carried out in the presence of increasing concentration of ‘6’.

FIG. 8: Virtual screening places the RAD52 inhibitors within the ssDNA binding groove. a) Three individual monomers of the RAD52-NTD undecameric ring (PDB 1KNO) are shown. ‘1’ and ‘6’ occupy similar sites at the interface of two subunits. Two dashed grey lines in each panel indicate the approximate boundaries of the ssDNA-binding groove. b). MOE ligand maps highlight water mediated interactions as well as interactions with amino acids. ‘1’ likely mediates interactions through R55, V128, E140, and E145, as well as through water contacts made with G59, M56, and K141. ‘6’ likely binds via hydrogen bonding via D149 and 1166 as well as through water interactions with E140, K144, and R153.

FIG. 9: Inhibiting the RAD52-ssDNA interaction interferes with RAD52/MUS81-mediated DSB formation essential for replication fork recovery in check point deficient cells. a) Representative images showing fields of cells from the comet assay for untreated, as well as from UCN01 (300 nM) and HU (2 mM) treated cells in the presence and absence of ‘1’, ‘6’, and siRAD52. b) ‘1’ and ‘6’ at 1 or 25 μM recapitulate RAD52 depletion. GM1604 cells, transfected or not with siRNAs against RAD52, were treated as indicated, in the presence or absence of the inhibitor. At the end of treatment, DSBs were analyzed by neutral comet assay. Data are presented as the mean±SEM from two independent experiments; p values are shown in the graph when differences are statistically significant. c-d) Inhibitors ‘1’ and ‘6’, respectively decrease the mean tail moment following HU treatment with IC₅₀ values ranging from mid nanomolar to low micromolar. Cells were treated as in (b). Data are from three independent experiments.

FIG. 10: Compound ‘1’ does not affect MUS81 activity. a) GM01604 wild-type fibroblasts were transfected with CTRL or MUS81 siRNA. Forty eight hours after transfection the fibroblasts were lysed and analysed by WB with the indicated antibodies. b) Forty eight hours after transfection, GM01604 cells were exposed to 0.204 aphidicolin (APH) for 24 h in the presence (grey bars) or absence (white bars) of increasing concentration of ‘1’ added in the last 3 h before aphidicolin treatment. Results are presented as mean±SEM from two independent replicates. c) The presence of anaphase bridges, as shown in the representative images, was scored in DAPI-stained cells. The white bars indicate 10 μm.

FIG. 11: Inhibition of ssDNA binding by RAD52 is sufficient to stimulate cell death in the absence of the MUS81 nuclease or BRCA2 tumor suppressor. a) The WB shows the analysis of RNAi. b) Evaluation of cell death after replication stress. Forty eight hours after transfection with the BRCA2 or MUS81 siRNAs, alone or in combination, the GM01604 cells were treated with compound ‘1’ or solvent (DMSO). Where indicated, the CHK1 inhibitor UCN-01 and HU was added and the cells were treated for 6 h, followed by 18 h of recovery in drug-free medium. Compound ‘1’ was present during the 6 h of treatment. Cell viability was evaluated by LIVE/DEAD assay as described in “Materials and Methods”. Data are presented as percentage of dead cells and are mean values from three independent experiments. Error bars represent SEM. The numbers shown in the graph represent the p value; the first p value of each pair refers to untreated cells while the second to the treated cells (2 way ANOVA). c) Representative images of live cells (green) and dead cells (red).

FIG. 12: Inhibition of ssDNA binding to RAD52 is sufficient to stimulate cell death in the absence of the BRCA2 tumor suppressor. a) Western blot analysis of BRCA2, RAD52, and GAPDH (loading control) protein levels in GM01604 cells treated with Ctrl, BRCA2, and RAD52 siRNAs. b) Evaluation of cell death after replication stress in cells treated with ‘1’. GM01604 cells were transfected with the RAD52 or BRCA2 siRNAs, alone or in combination, and 48 h thereafter treated as indicated. The ‘1’ inhibitor or solvent (DMSO) was added to media 1 h prior to replication stress. Cell viability was evaluated by LIVE/DEAD assay as described in the “Materials and Methods”. Data are presented as percentage of dead cells and are mean values from three independent experiments. Error bars represent standard error. The numbers shown in the graph represent the p value; the first p value of each pair refers to untreated cells while the second to the treated cells (2 way ANOVA). c) Representative images: live cells are stained green, while dead cells are red.

FIG. 13: In silico screening campaign identifies novel small molecule that inhibits the RAD52-ssDNA interaction. a) Docking workflow involved RAD52-NTD undecameric ring (PDB 1KNO) pre-processing, AnalytiCon Discovery MEGx Natural Products Screening Library pre-processing, and classical docking using the Dock utility of MOE. Top ranking poses (those with the lowest energy scores from the London dG scoring function) were subjected to a refining docking step involving force field-based energy minimization. From these complexes, predicted binding free energies were calculated. Workflow validation involved RAD52-NTD pre-processing, pre-processing of ‘1’ and associated decoys (DUD-E), followed by classical docking and score ranking. ROC curves were then generated and analyzed. Scores of the conformations of inhibitor compounds and of conformations of their respective decoy compounds were compared. b) Docking scores (kcal/mol) for the individual conformations of compound ‘1’ and decoy compounds were binned and plotted as histograms. The low docking scores, as indicated by the more negative predicted free energies of ‘1’ when compared to decoys, indicate more favorable poses, and highlight a distinct separation between true positives and true negatives. c) Receiving-operating characteristic (ROC) curve shows that the classifier used, i.e. the scoring function, was close to optimal in distinguishing compound ‘1’ conformations from those of decoys confirmed by AUC analysis yielding a value of 0.9973. d) Electrostatic surface potential of three monomers of the RAD52-NTD undecameric ring (PDB 1KNO) depicting NP-004255 within the ssDNA binding groove. e). MOE ligand maps highlight water mediated interactions with E145 and D149 as well as via hydrogen bonding with amino acids R55.

FIG. 14: Biochemical characterization of NP-004255. a) Aromatic region of the 1D ¹H NMR spectrum of compound NP-004255 alone (top line) and the WaterLOGSY spectrum of 40 μM compound NP-004255 in the presence of 3.3 μM RAD52 (bottom line). The nonexchangeable proton peaks are labeled using atom names as indicated on the structure of compound NP-004255. b) IC₅₀ values for inhibition of ssDNA binding and wrapping were determined using FRET-based assays that follow the change in geometry of a Cy3-dT30-Cy5 substrate (black circles). Computed IC₅₀ value is shown above the curve. Titration of the RAD52-dsDNA with NP-004255 (grey boxes) shows that this inhibitor does not perturb the RAD52-dsDNA interaction. c) Aromatic region of the 1D ¹H NMR spectrum of compound NP-004255 alone (top line) and the WaterLOGSY spectrum of 40 μM compound NP-004255 in the presence of 3.3 μM RPA (bottom line). d) Titration of the RAD52-RPA-Cy3-dT₃₀-Cy5 complex with NP-004255 (black circles). Computed IC₅₀ value is shown below the curve. Bottom line shows titration of the RPA-Cy3-dT₃₀-Cy5 complex with NP-004255 indicating NP-004255 does not perturb the RPA-ssDNA interaction.

### DETAILED DESCRIPTION

Disclosed are methods, compositions, kits, and systems for identifying small-molecule drugs for treating cancer in a subject. The disclosed methods, compositions, kits, and systems may be utilized to identify small-molecule inhibitors of radiation sensitive protein 52 (RAD52) in order to treat cancer in a subject, such as breast cancer in a subject having a BRCA1-deficient, BRCA2-deficient, and/or PALB2-deficient phenotype. The disclosed methods, compositions, kits, and systems may be further described and defined as follows.

Unless otherwise specified or indicated by context, the terms “a”, “an”, and “the” mean “one or more.” In addition, singular nouns such as “RAD52 inhibitor” should be interpreted to mean “one or RAD52 inhibitors.”

As used herein, “about”, “approximately,” “substantially,” and “significantly” will be understood by persons of ordinary skill in the art and will vary to some extent on the context in which they are used. If there are uses of the term which are not clear to persons of ordinary skill in the art given the context in which it is used, “about” and “approximately” will mean plus or minus ≤10% of the particular term and “substantially” and “significantly” will mean plus or minus ≥10% of the particular term.

As used herein, the terms “include” and “including” have the same meaning as the terms “comprise” and “comprising.” The terms “comprise” and “comprising” should be interpreted as being “open” transitional terms that permit the inclusion of additional components further to those components recited in the claims. The terms “consist” and “consisting of” should be interpreted as being “closed” transitional terms that do not permit the inclusion of additional components other than the components recited in the claims. The term “consisting essentially of” should be interpreted to be partially closed and allowing the inclusion only of additional components that do not fundamentally alter the nature of the claimed subject matter.

The terms “subject,” “patient,” or “host” may be used interchangeably herein and may refer to human or non-human animals. Non-human animals may include, but are not limited to non-human primates, dogs, cats, and mice.

The terms “subject,” “patient,” or “individual” may be used to a human or non-human animal having or at risk for acquiring a cell proliferative disease or disorder. Individuals who are treated with the compositions disclosed herein may be at risk for cancer or may have already acquired cancer including cancers associated with RAD52 biological activity including breast cancer and in particular breast cancer in a subject having a BRCA1-deficient, BRCA2-deficient, and/or PALB2-deficient phenotype. Other cancers that may be associated with RAD52 biological activity may include but are not limited to adenocarcinoma, lymphoma, melanoma, myeloma, sarcoma, and teratocarcinoma and particularly cancers of the adrenal gland, bladder, bone, bone marrow, brain, cervix, gall bladder, ganglia, gastrointestinal tract, heart, kidney, liver, lung, muscle, ovary, pancreas, parathyroid, prostate, skin, testis, thymus, and uterus.

The terms “subject,” “patient,” or “individual” may be a human having breast and a BRCA1, BRCA2, or PALB2 deficient phenotype, as understood in the art. (See Prakash R., ZHANG, Y., FENG, W. & JASIN, M. 2015. Homologous Recombination and Human Health: The Roles of BRCA1, BRCA2, and Associated Proteins. Cold Spring Harbor Perspectives in Biology, 7, the content of which is incorporated herein by reference in its entirety). A subject that is deficient in BRCA1, BRCA2, or PALB2 may include a subject having one or more mutations in BRCA1, BRCA2, or PALB2 that render the encoded protein product of BRCA1, BRCA2, or PALB2 non-expressed, defective, or non-operable. A subject that is deficient in BRCA1, BRCA2, or PALB2 may include a subject that is homozygous for one or mutations in BRCA1, BRCA2, or PALB2 that render the encoded protein product of BRCA1, BRCA2, or PALB2 non-expressed, defective, or non-operable.

Compounds and uses thereof are disclosed herein. The disclosed compounds may be referred to as “small-molecule compounds.” In referring to the compounds disclosed herein, the term “alkyl” includes a straight-chain or branched alkyl radical in all of its isomeric forms. Similarly, the term “alkoxy” refers to any alkyl radical which is attached via an oxygen atom (i.e., a radical represented as “alkyl-O—*”). As used herein, an asterisk “*” or plus sign “+” is used to designate the point of attachment for any radical group or substituent group.

As used herein, the phrase “effective amount” shall mean that drug dosage of a compound that provides the specific pharmacological response for which the drug is administered in a significant number of patients in need of such treatment. An effective amount of a drug that is administered to a particular patient in a particular instance will not always be effective in treating the conditions/diseases described herein, even though such dosage is deemed to be a therapeutically effective amount by those of skill in the art.

The present application relates to Radiation Sensitive Protein 52 [Homo sapiens] (RAD52), which may have the following amino acid sequence (GenBank: AAA85793.1) (SEQ ID NO:1):

| | |
|---|---|
| 1 | msgteeailg grdshpaagg gsvlcfgqcq ytaeeyqaiq kalrqrlgpe yissrmaggg |
| 61 | qkvcyieghr vinlanemfg yngwahsitq qnvdfvdlnn gkfyvgvcaf vrvqlkdgsy |
| 121 | hedvgygvse glkskalsle karkeavtdg lkralrsfgn algncildkd ylrslnklpr |
| 181 | qlplevdltk akrqdlepsv eearynscrp nmalghpqlq qvtspsrpsh avipadqdcs |
| 241 | srslsssave seathqrklr qkqlqqqfre rmekqqvrvs tpsaekseaa ppappvthst |
| 301 | pvtvseplle kdflagvtqe liktlednse kwavtpdagd gvvkpssrad paqtsdtlal |
| 361 | nnqmvtqnrt phsvchqkpq aksgswdlqt ysadqrttgn weshrksqdm kkrkydps |

As would be understood in the art, RAD52 has biological activities that include, but are not limited to, homo-oligomerization, binding to ssDNA (optionally in the presence of Recombinase protein A (RPA)), and annealing of ssDNA (optionally in the presence of RPA)).

The disclosed compounds may modulate the biological activity of RAD52. As used herein, the term “modulate” means decreasing or inhibiting activity and/or increasing or augmenting activity. For example, modulating RAD52 biological activity means decreasing or inhibiting RAD52 biological activity and/or increasing or augmenting RAD52 biological activity. The compounds disclosed herein may be administered to modulate RAD52 biological activity for example, as an inhibitor, a chaperone, or an activator. Preferably, the disclosed compounds inhibit one or more biological activities of RAD52.

The compounds disclosed herein may have several chiral centers, and stereoisomers, epimers, and enantiomers are contemplated. In the formulas disclosed herein, unless indicated, a formula should be interpreted to encompass all stereoisomers, epimers, and enantiomers of the formula. The compounds may be optically pure with respect to one or more chiral centers (e.g., some or all of the chiral centers may be completely in the S configuration; some or all of the chiral centers may be completely in the R configuration; etc.). Additionally or alternatively, one or more of the chiral centers may be present as a mixture of configurations (e.g., a racemic or another mixture of the R configuration and the S configuration). Compositions comprising substantially purified stereoisomers, epimers, or enantiomers, or analogs or derivatives thereof are contemplated herein (e.g., a composition comprising at least about 90%, 95%, or 99% pure stereoisomer, epimer, or enantiomer.) As used herein, formulae which do not specify the orientation at one or more chiral centers are meant to encompass all orientations and mixtures thereof.

The compounds employed in the compositions and methods disclosed herein may be administered as pharmaceutical compositions and, therefore, pharmaceutical compositions incorporating the compounds are considered to be embodiments of the compositions disclosed herein. Such compositions may take any physical form which is pharmaceutically acceptable; illustratively, they can be orally administered pharmaceutical compositions. Such pharmaceutical compositions contain an effective amount of a disclosed compound, which effective amount is related to the daily dose of the compound to be administered. Each dosage unit may contain the daily dose of a given compound or each dosage unit may contain a fraction of the daily dose, such as one-half or one-third of the dose. The amount of each compound to be contained in each dosage unit can depend, in part, on the identity of the particular compound chosen for the therapy and other factors, such as the indication for which it is given. The pharmaceutical compositions disclosed herein may be formulated so as to provide quick, sustained, or delayed release of the active ingredient after administration to the patient by employing well known procedures.

The compounds for use according to the methods of disclosed herein may be administered as a single compound or a combination of compounds. For example, a compound that modulates RAD52 biological activity may be administered as a single compound or in combination with another compound that modulates RAD52 biological activity or that has a different pharmacological activity.

As indicated above, pharmaceutically acceptable salts of the compounds are contemplated and also may be utilized in the disclosed methods. The term “pharmaceutically acceptable salt” as used herein, refers to salts of the compounds which are substantially non-toxic to living organisms. Typical pharmaceutically acceptable salts include those salts prepared by reaction of the compounds as disclosed herein with a pharmaceutically acceptable mineral or organic acid or an organic or inorganic base. Such salts are known as acid addition and base addition salts. It will be appreciated by the skilled reader that most or all of the compounds as disclosed herein are capable of forming salts and that the salt forms of pharmaceuticals are commonly used, often because they are more readily crystallized and purified than are the free acids or bases.

Acids commonly employed to form acid addition salts may include inorganic acids such as hydrochloric acid, hydrobromic acid, hydroiodic acid, sulfuric acid, phosphoric acid, and the like, and organic acids such as p-toluenesulfonic, methanesulfonic acid, oxalic acid, p-bromophenylsulfonic acid, carbonic acid, succinic acid, citric acid, benzoic acid, acetic acid, and the like. Examples of suitable pharmaceutically acceptable salts may include the sulfate, pyrosulfate, bisulfate, sulfite, bisulfate, phosphate, monohydrogenphosphate, dihydrogenphosphate, metaphosphate, pyrophosphate, bromide, iodide, acetate, propionate, decanoate, caprylate, acrylate, formate, hydrochloride, dihydrochloride, isobutyrate, caproate, heptanoate, propiolate, oxalate, malonate, succinate, suberate, sebacate, fumarate, maleat-, butyne-.1,4-dioate, hexyne-1,6-dioate, benzoate, chlorobenzoate, methylbenzoate, hydroxybenzoate, methoxybenzoate, phthalate, xylenesulfonate, phenylacetate, phenylpropionate, phenylbutyrate, citrate, lactate, alpha-hydroxybutyrate, glycolate, tartrate, methanesulfonate, propanesulfonate, naphthalene-l-sulfonate, naphthalene-2-sulfonate, mandelate, and the like.

Base addition salts include those derived from inorganic bases, such as ammonium or alkali or alkaline earth metal hydroxides, carbonates, bicarbonates, and the like. Bases useful in preparing such salts include sodium hydroxide, potassium hydroxide, ammonium hydroxide, potassium carbonate, sodium carbonate, sodium bicarbonate, potassium bicarbonate, calcium hydroxide, calcium carbonate, and the like.

The particular counter-ion forming a part of any salt of a compound disclosed herein is may not be critical to the activity of the compound, so long as the salt as a whole is pharmacologically acceptable and as long as the counterion does not contribute undesired qualities to the salt as a whole. Undesired qualities may include undesirably solubility or toxicity.

Pharmaceutically acceptable esters and amides of the compounds can also be employed in the compositions and methods disclosed herein. Examples of suitable esters include alkyl, aryl, and aralkyl esters, such as methyl esters, ethyl esters, propyl esters, dodecyl esters, benzyl esters, and the like. Examples of suitable amides include unsubstituted amides, monosubstituted amides, and disubstituted amides, such as methyl amide, dimethyl amide, methyl ethyl amide, and the like.

In addition, the methods disclosed herein may be practiced using solvate forms of the compounds or salts, esters, and/or amides, thereof. Solvate forms may include ethanol solvates, hydrates, and the like.

The pharmaceutical compositions may be utilized in methods of treating a disease or disorder associated with RAD52 biological activity. For example, the pharmaceutical compositions may be utilized to treat patients having or at risk for acquiring a proliferative disease or disorder such as cancer, and in particular, breast cancer (e.g., BRCA1-deficient, BRCA2-deficient, and/or PALB2-deficient breast cancer).

As used herein, the terms “treating” or “to treat” each mean to alleviate symptoms, eliminate the causation of resultant symptoms either on a temporary or permanent basis, and/or to prevent or slow the appearance or to reverse the progression or severity of resultant symptoms of the named disease or disorder. As such, the methods disclosed herein encompass both therapeutic and prophylactic administration.

As used herein the term “effective amount” refers to the amount or dose of the compound, upon single or multiple dose administration to the subject, which provides the desired effect in the subject under diagnosis or treatment. The disclosed methods may include administering an effective amount of the disclosed compounds (e.g., as present in a pharmaceutical composition) for treating a disease or disorder associated with RAD52 biological activity.

An effective amount can be readily determined by the attending diagnostician, as one skilled in the art, by the use of known techniques and by observing results obtained under analogous circumstances. In determining the effective amount or dose of compound administered, a number of factors can be considered by the attending diagnostician, such as: the species of the subject; its size, age, and general health; the degree of involvement or the severity of the disease or disorder involved; the response of the individual subject; the particular compound administered; the mode of administration; the bioavailability characteristics of the preparation administered; the dose regimen selected; the use of concomitant medication; and other relevant circumstances.

A typical daily dose may contain from about 0.01 mg/kg to about 100 mg/kg (such as from about 0.05 mg/kg to about 50 mg/kg and/or from about 0.1 mg/kg to about 25 mg/kg) of each compound used in the present method of treatment.

Compositions can be formulated in a unit dosage form, each dosage containing from about 1 to about 500 mg of each compound individually or in a single unit dosage form, such as from about 5 to about 300 mg, from about 10 to about 100 mg, and/or about 25 mg. The term “unit dosage form” refers to a physically discrete unit suitable as unitary dosages for a patient, each unit containing a predetermined quantity of active material calculated to produce the desired therapeutic effect, in association with a suitable pharmaceutical carrier, diluent, or excipient.

Oral administration is an illustrative route of administering the compounds employed in the compositions and methods disclosed herein. Other illustrative routes of administration include transdermal, percutaneous, intravenous, intramuscular, intranasal, buccal, intrathecal, intracerebral, or intrarectal routes. The route of administration may be varied in any way, limited by the physical properties of the compounds being employed and the convenience of the subject and the caregiver.

As one skilled in the art will appreciate, suitable formulations include those that are suitable for more than one route of administration. For example, the formulation can be one that is suitable for both intrathecal and intracerebral administration. Alternatively, suitable formulations include those that are suitable for only one route of administration as well as those that are suitable for one or more routes of administration, but not suitable for one or more other routes of administration. For example, the formulation can be one that is suitable for oral, transdermal, percutaneous, intravenous, intramuscular, intranasal, buccal, and/or intrathecal administration but not suitable for intracerebral administration.

The inert ingredients and manner of formulation of the pharmaceutical compositions are conventional. The usual methods of formulation used in pharmaceutical science may be used here. All of the usual types of compositions may be used, including tablets, chewable tablets, capsules, solutions, parenteral solutions, intranasal sprays or powders, troches, suppositories, transdermal patches, and suspensions. In general, compositions contain from about 0.5% to about 50% of the compound in total, depending on the desired doses and the type of composition to be used. The amount of the compound, however, is best defined as the “effective amount”, that is, the amount of the compound which provides the desired dose to the patient in need of such treatment. The activity of the compounds employed in the compositions and methods disclosed herein are not believed to depend greatly on the nature of the composition, and, therefore, the compositions can be chosen and formulated primarily or solely for convenience and economy.

Capsules are prepared by mixing the compound with a suitable diluent and filling the proper amount of the mixture in capsules. The usual diluents include inert powdered substances (such as starches), powdered cellulose (especially crystalline and microcrystalline cellulose), sugars (such as fructose, mannitol and sucrose), grain flours, and similar edible powders.

Tablets are prepared by direct compression, by wet granulation, or by dry granulation. Their formulations usually incorporate diluents, binders, lubricants, and disintegrators (in addition to the compounds). Typical diluents include, for example, various types of starch, lactose, mannitol, kaolin, calcium phosphate or sulfate, inorganic salts (such as sodium chloride), and powdered sugar. Powdered cellulose derivatives can also be used. Typical tablet binders include substances such as starch, gelatin, and sugars (e.g., lactose, fructose, glucose, and the like). Natural and synthetic gums can also be used, including acacia, alginates, methylcellulose, polyvinylpyrrolidine, and the like. Polyethylene glycol, ethylcellulose, and waxes can also serve as binders.

Tablets can be coated with sugar, e.g., as a flavor enhancer and sealant. The compounds also may be formulated as chewable tablets, by using large amounts of pleasant-tasting substances, such as mannitol, in the formulation. Instantly dissolving tablet-like formulations can also be employed, for example, to assure that the patient consumes the dosage form and to avoid the difficulty that some patients experience in swallowing solid objects.

A lubricant can be used in the tablet formulation to prevent the tablet and punches from sticking in the die. The lubricant can be chosen from such slippery solids as talc, magnesium and calcium stearate, stearic acid, and hydrogenated vegetable oils.

Tablets can also contain disintegrators. Disintegrators are substances that swell when wetted to break up the tablet and release the compound. They include starches, clays, celluloses, algins, and gums. As further illustration, corn and potato starches, methylcellulose, agar, bentonite, wood cellulose, powdered natural sponge, cation-exchange resins, alginic acid, guar gum, citrus pulp, sodium lauryl sulfate, and carboxymethylcellulose can be used.

Compositions can be formulated as enteric formulations, for example, to protect the active ingredient from the strongly acid contents of the stomach. Such formulations can be created by coating a solid dosage form with a film of a polymer which is insoluble in acid environments and soluble in basic environments. Illustrative films include cellulose acetate phthalate, polyvinyl acetate phthalate, hydroxypropyl methylcellulose phthalate, and hydroxypropyl methylcellulose acetate succinate.

As one skilled in the art will also appreciate, the formulation can be prepared with materials (e.g., actives excipients, carriers (such as cyclodextrins), diluents, etc.) having properties (e.g., purity) that render the formulation suitable for administration to humans. Alternatively, the formulation can be prepared with materials having purity and/or other properties that render the formulation suitable for administration to non-human subjects, but not suitable for administration to humans.

Methods for Treating Cancers Associated with RAD52 Biological Activity

Disclosed are methods for treating cancers. Particularly disclosed are methods for treating cancers that are associated with RAD52 biological activity. The disclosed methods typically include administering a therapeutic agent that inhibits the biological activity of RAD52.

The disclosed methods may be practiced for treating breast cancer in a subject in need thereof. Suitable breast cancers that may be treated by the methods may include, but are not limited to BRCA1-deficient breast cancer, BRCA2-deficient breast cancer, and PALB2-deficient cancer.

The therapeutic agent that is administered in the disclosed methods typically inhibits one or more biological activities of RAD52. In some embodiments, the therapeutic agent binds to RAD52, preferably with a K_{D} of less than about 100, 50, 20, 10, 5, 2, 1, 0.5, 0.2, 0.1, 0.05, 0.02, or 0.01 μM. In some embodiments, the therapeutic agent inhibits binding between RAD52 and ssDNA, preferably with an IC₅₀ of less than about 100, 50, 20, 10, 5, 2, 1, 0.5, 0.2, 0.1, 0.05, 0.02, or 0.01 μM. In some embodiments, the therapeutic agent inhibits binding between RAD52 and ssDNA that is coated with Replication protein A (RPA) (i.e., RPA-coated ssDNA), preferably with an IC₅₀ of less than about 100, 50, 20, 10, 5, 2, 1, 0.5, 0.2, 0.1, 0.05, 0.02, or 0.01 μM. In some embodiments, the therapeutic agent inhibits RNA52 annealing of ssDNA (e.g., to another strand of ssDNA), preferably with an IC₅₀ of less than about 100, 50, 20, 10, 5, 2, 1, 0.5, 0.2, 0.1, 0.05, 0.02, or 0.01 μM. In some embodiments, the therapeutic agent inhibits RAD52 annealing of RPA-coated ssDNA, preferably with an IC₅₀ of less than about 100, 50, 20, 10, 5, 2, 1, 0.5, 0.2, 0.1, 0.05, 0.02, or 0.01 μM.

The therapeutic agent utilized in the disclosed methods may be referred to as a “small molecule compound.” In some embodiments, the small molecule compound is selected from the following compounds, hydrates thereof, or pharmaceutically acceptable salts thereof:

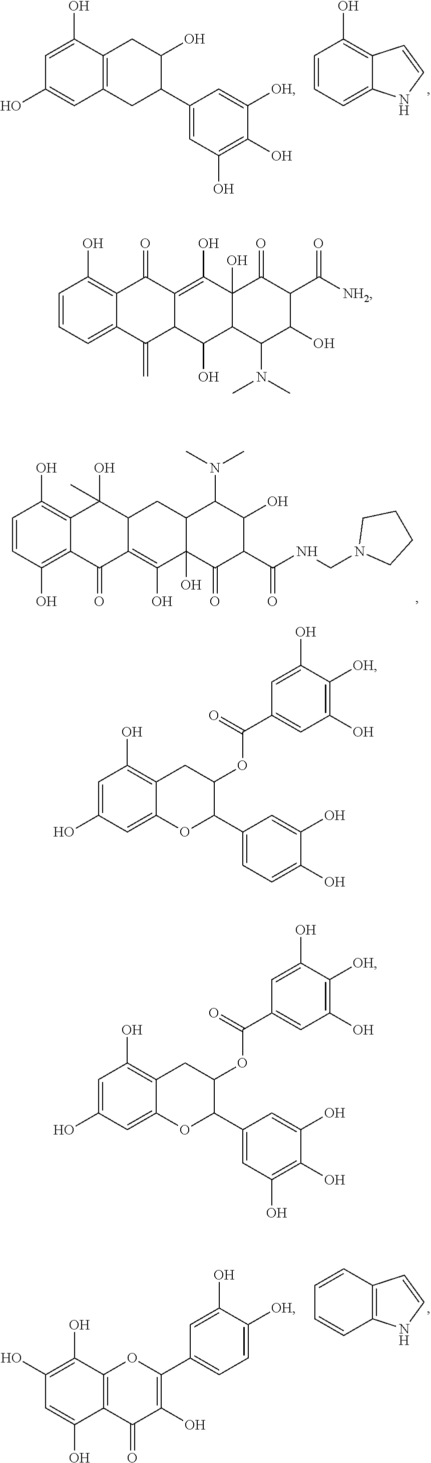

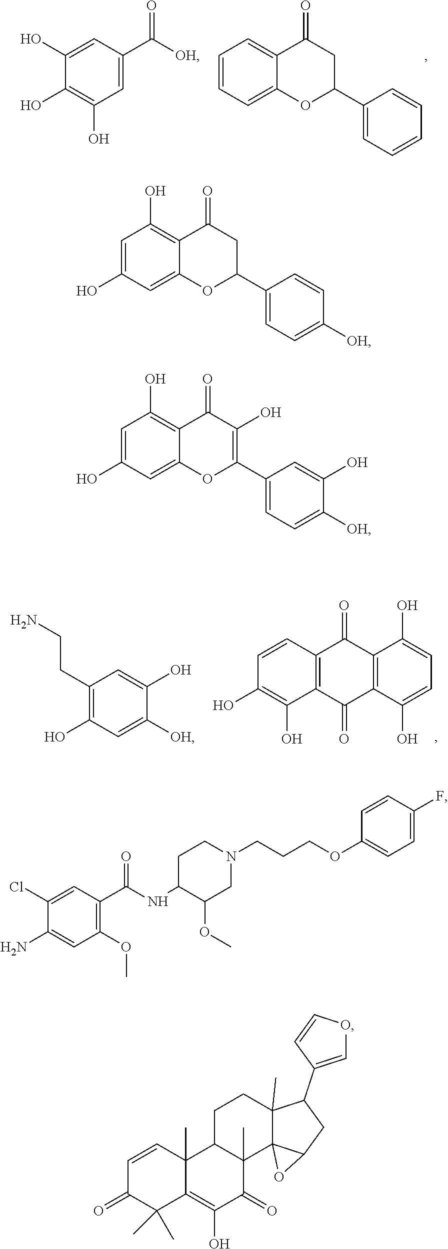

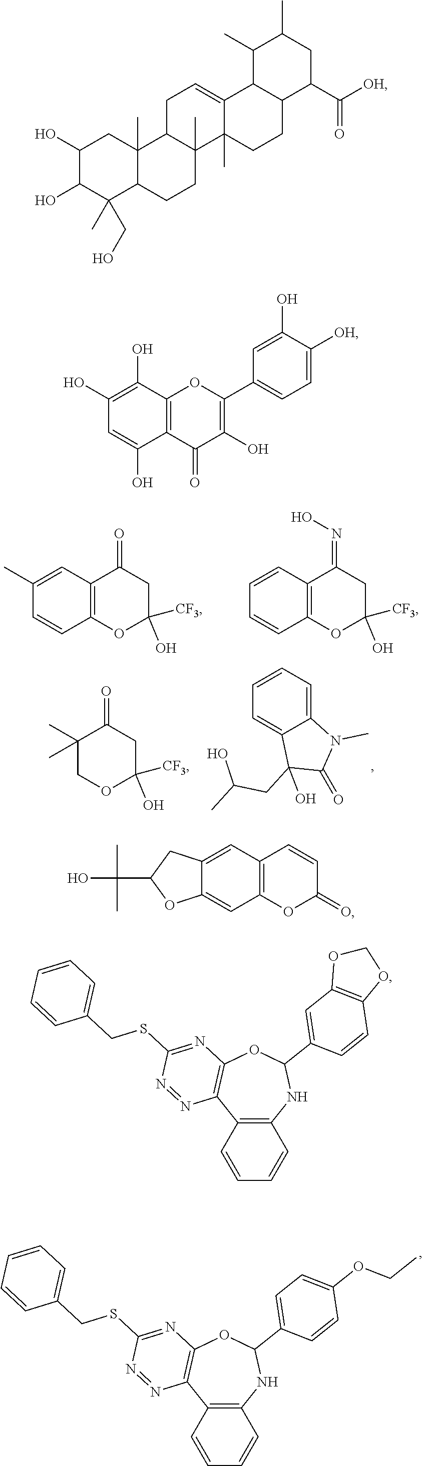

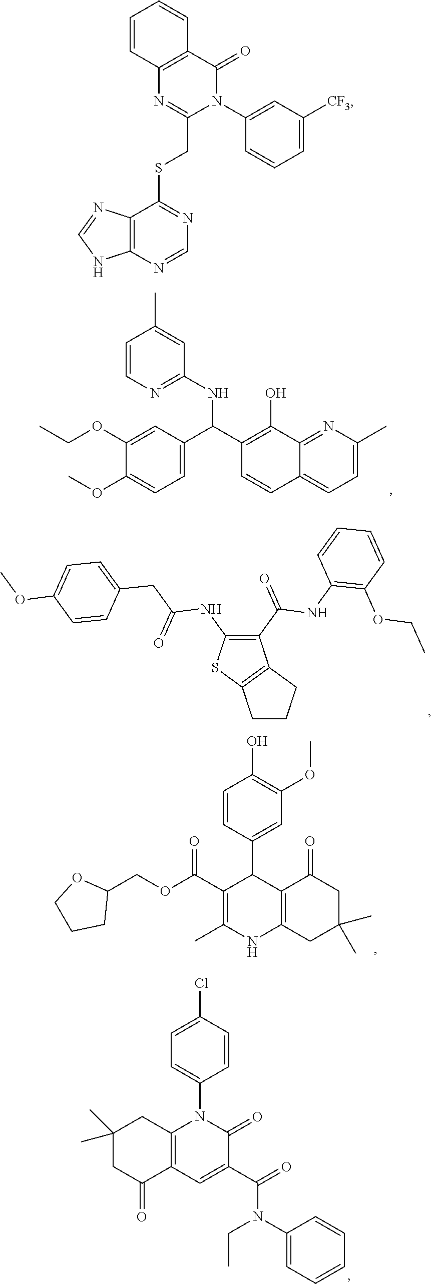

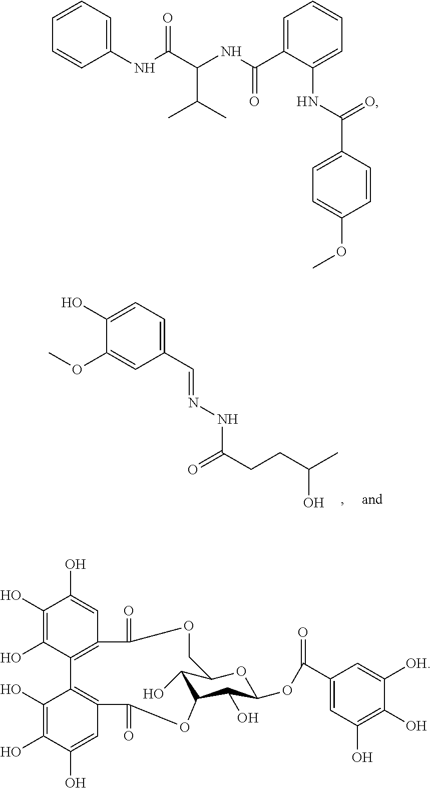 [SMILES: COc1ccc(C(=O)Nc2ccccc2C(=O)NC(C(=O)Nc2ccccc2)C(C)C)cc1 ; COc1cc(/C=N/NC(=O)CCC(C)O)ccc1O ; O=C(O[C@@H]1OC2COC(=O)c3cc(O)c(O)c(O)c3-c3c(cc(O)c(O)c3O)C(=O)O[C@H](C1O)[C@@H]2O)c1cc(O)c(O)c(O)c1]

In further embodiments of the disclosed methods, the therapeutic agent administered in the methods is a compound having a structure:

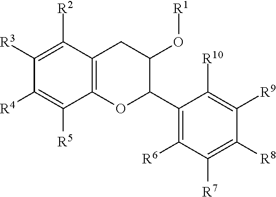

wherein R¹ is H, C1-C6 alkyl, C1-C6 alkoxy, or

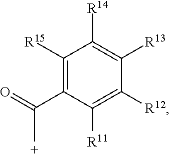

wherein R¹¹, R¹², R¹³, R¹⁴, and R¹⁵ are each independently selected from H, —OH, halo, C1-C6 alkyl, and C1-C6 alkoxy; and R², R³, R⁴, R⁵, R⁶, R⁷, R⁸, R⁹ and R¹⁰ are each independently selected from H, —OH, halo, C1-C6 alkyl, and C1-C6 alkoxy.

In particular, the therapeutic agent administered in the disclosed methods may be a compound having a formula:

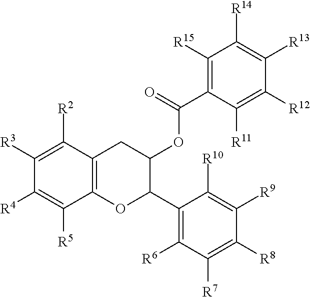

where R², R³, R⁴, R⁵, R⁶, R⁷, R⁸, R⁹, R¹⁰, R¹¹, R¹², R¹³, R¹⁴ and R¹⁵ are as described above.

In further embodiments of the disclosed methods, the therapeutic agent administered in the methods is a compound selected from the group consisting of (−)-epigallocatechin, (−)-epicatechin gallate; epigallocatechin-3-monogallate, quercetin, naringenin, taxifolin, fisetin, myricetin, tricetin, cyanidin, eriodictyol, 3-methylquercetin, robinetin, tamarixetin, epiafzelechin, 3′-O-methylepicatechin, meciadanol, theaflavin, 5,7,3′-trihydroxy-3,4′-dimethoxyflavone, 2H-1-benzopyran-3,7-dio1,2-(3,4-dihydroxyphenyl)-3,4-dihydro-, petunidin, 4′-methylepigallocatechin, delphinidin, (+)-Epicatechin, taxifolin, mearnsetin, Fisetin 3-methyl ether, 7,3′,4′,5′-Tetrahydroxyflavone, 3,5,7,4′-Tetrahydroxyflavan, fustin, leucocyanidin, melacacidin, ampelopsin, cyrtominetin, (−)-Gallocatechin, 2H-1-Benzopyran-5,7-diol, 3,4-dihydro-2-(4-hydroxyphenyl)-, robinetinidol, 3′-O-methylcatechin, Epicatechin 3′,4′-dimethyl ether, leukoefdin, 2-(3,4-dihydroxyphenyl)-3,5,7-trihydroxy-2,3-dihydronaphthalene-1,4-dione, flavan-3-ol, luteoforol, 7,4′-Dihydroxyflavan, luteoforol, leukoefdin, afzelechin, Fisetinidol, Apiforol, Dihydrokaempferide, leukoefdin, Laricitrin, 5,7,4′-Tri-O-methylcatechin, (?)-Epicatechin quione, 4H-1-Benzopyran-4-one, 5,7,8-trihydroxy-2-(3,4,5-trihydroxyphenyl)-, 3′-Hydroxy-4′-O-methylglabridin, Mesquitol, Tricetinidin, (+)-Epiaromadendrin, L-Epicatechin, 1,2-Benzenediol, 4-(3,4-dihydro-7-hydroxy-2H-1-benzopyran-2-yl)-, Pinomyricetin, Epidistenin, 4′-O-methyepicatechin, Hibiscetin, Epimesquitol-4beta-ol, 4H-1-Benzopyran-4-one, 5,7-dihydroxy-2-(4-hydroxyphenyl)-3-methyl-, 4H-1-Benzopyran-4-one, 5-hydroxy-2-(3,4,5-trihydroxyphenyl)-, 2,3-Dihydrogossypetin, 2-(4-hydroxyphenyl)-3,4-dihydro-2h-chromene-4,5,7-triol, epi-Catechol, (2S)-dihydrotricetin, Taxifolin 3-O-acetate, Arachidoside, Leuco-fisetinidin, 3,5,7-trihydroxy-2-(3,4,5-trihydroxyphenyl)-3,4-dihydro-2H-1-benzopyran-4-one, “Isoetin, Guibourtinidol, 4′-O-Methylcatechin, Epicatechin 5,3′-dimethyl ether, 3-O-Methylepicatechin, Keto-teracacidin, Apigeniflavan, 3,5,8,3′,4′,5′-and Hexahydroxyflavone.

Also disclosed are pharmaceutical compositions comprising as a therapeutic agent a compound selected from the following compounds, hydrates thereof, or pharmaceutically acceptable salts thereof:

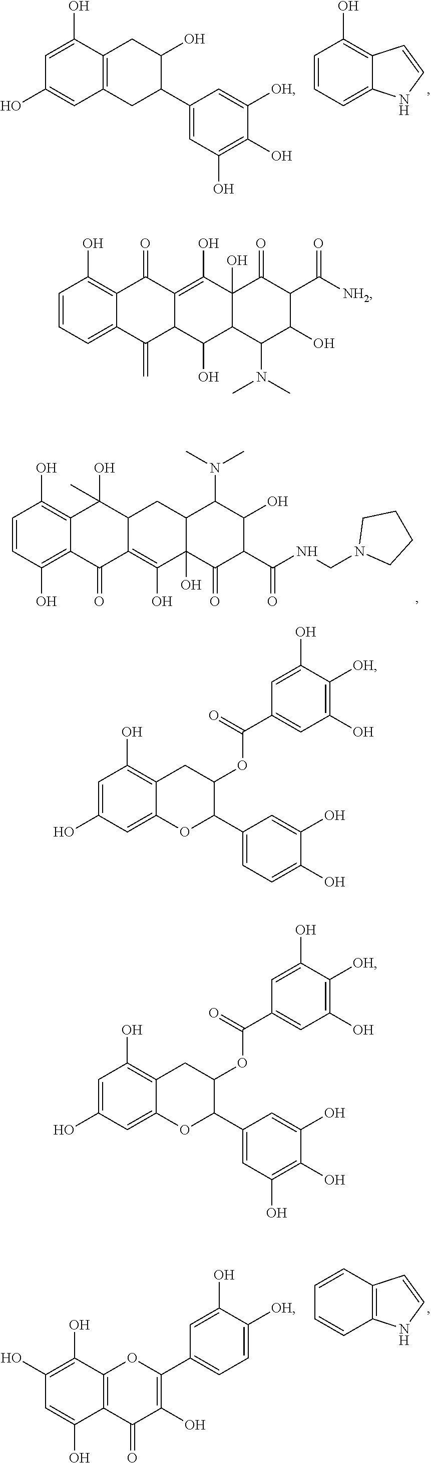

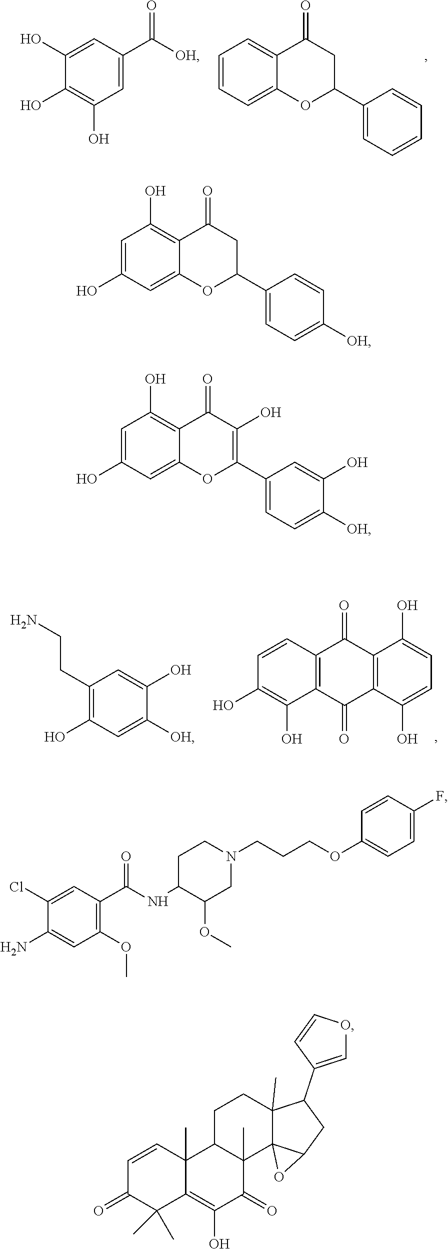

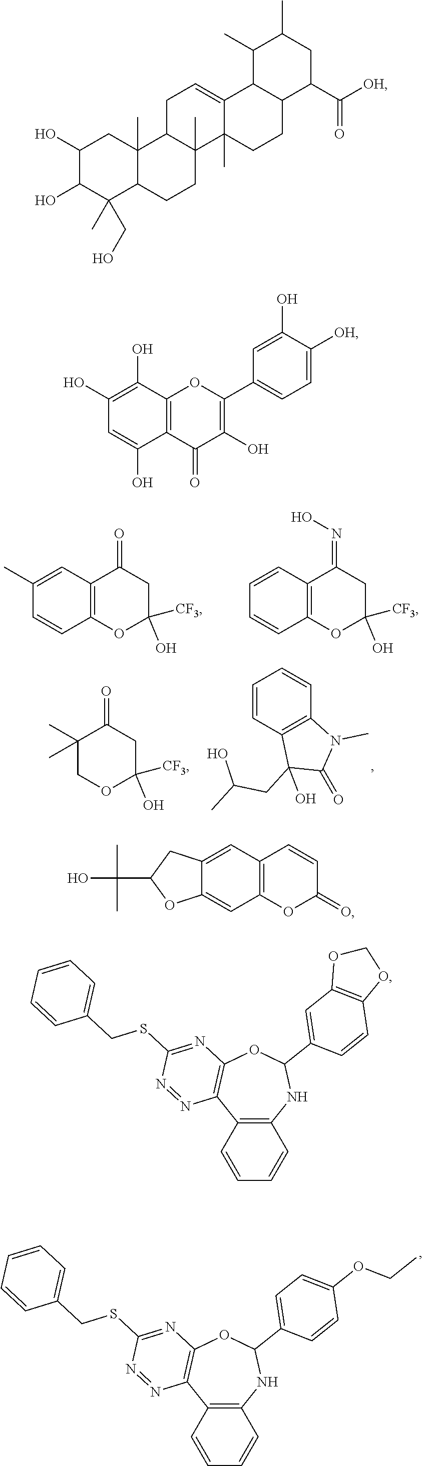

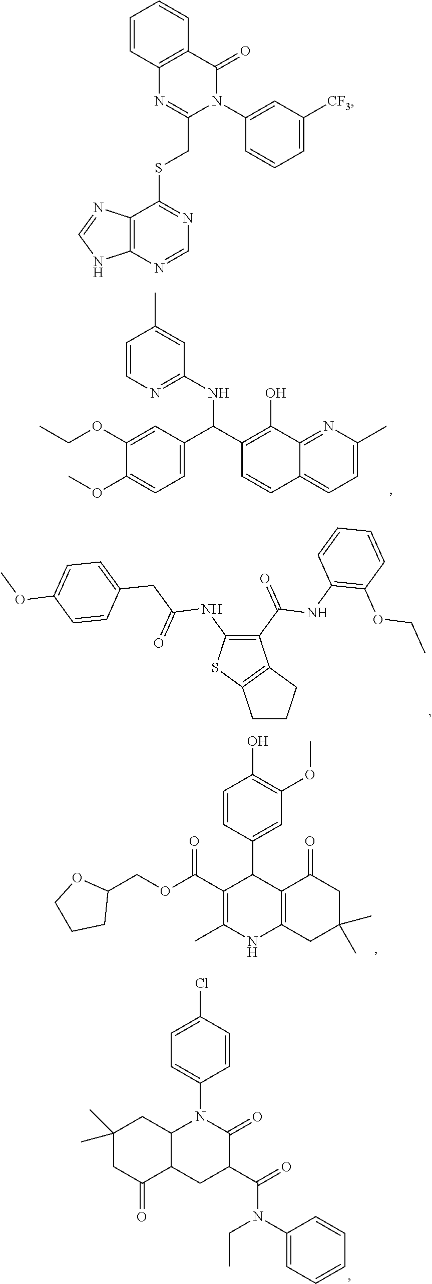

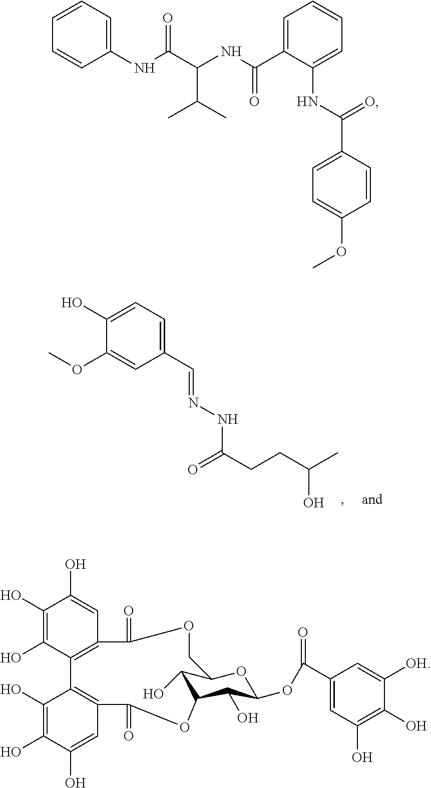 [SMILES: COc1ccc(C(=O)Nc2ccccc2C(=O)NC(C(=O)Nc2ccccc2)C(C)C)cc1 ; COc1cc(/C=N/NC(=O)CCC(C)O)ccc1O ; O=C(O[C@@H]1OC2COC(=O)c3cc(O)c(O)c(O)c3-c3c(cc(O)c(O)c3O)C(=O)O[C@H](C1O)[C@@H]2O)c1cc(O)c(O)c(O)c1]

In further embodiments, the pharmaceutical compositions may comprise as a therapeutic agent a compound selected from the following compounds, hydrates thereof, or pharmaceutically acceptable salts thereof:

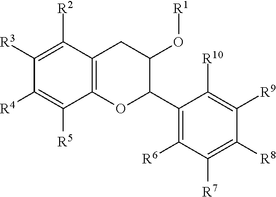

wherein R¹ is H, C1-C6 alkyl, C1-C6 alkoxy, or

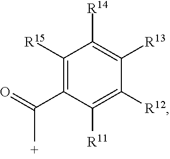

wherein R¹¹, R¹², R¹³, R¹⁴, and R¹⁵ are each independently selected from H, —OH, halo, C1-C6 alkyl, and C1-C6 alkoxy; and
R², R³, R⁴, R⁵, R⁶, R⁷, R⁸, R⁹ and R¹⁰ are each independently selected from H, —OH, halo, C1-C6 alkyl, and C1-C6 alkoxy.

In further embodiments, the pharmaceutical compositions comprise a compound having a formula:

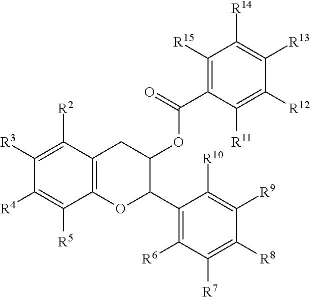

wherein R², R³, R⁴, R⁵, R⁶, R⁷, R⁸, R⁹, R¹⁰, R¹¹, R¹², R¹³, R¹⁴, and R¹⁵ are as described above.

In further embodiments, the pharmaceutical compositions comprise as a therapeutic agent a compound selected from the following compounds, hydrates thereof, or pharmaceutically acceptable salts thereof:method of any of the foregoing claims wherein the compound is selected from the group consisting of (−)-epigallocatechin, (−)-epicatechin gallate; epigallocatechin-3-monogallate, quercetin, naringenin, taxifolin, fisetin, myricetin, tricetin, cyanidin, eriodictyol, 3-methylquercetin, robinetin, tamarixetin, epiafzelechin, 3′-O-methylepicatechin, meciadanol, theaflavin, 5,7,3′-trihydroxy-3,4′-dimethoxyflavone, 2H-1-benzopyran-3,7-dio1,2-(3,4-dihydroxyphenyl)-3,4-dihydro-, petunidin, 4′-methylepigallocatechin, delphinidin, (+)-Epicatechin, taxifolin, mearnsetin, Fisetin 3-methyl ether, 7,3′,4′,5′-Tetrahydroxyflavone, 3,5,7,4′-Tetrahydroxyflavan, fustin, leucocyanidin, melacacidin, ampelopsin, cyrtominetin, (−)-Gallocatechin, 2H-1-Benzopyran-5,7-diol, 3,4-dihydro-2-(4-hydroxyphenyl)-, robinetinidol, 3′-O-methylcatechin, Epicatechin 3′,4′-dimethyl ether, leukoefdin, 2-(3,4-dihydroxyphenyl)-3,5,7-trihydroxy-2,3-dihydronaphthalene-1,4-dione, flavan-3-ol, luteoforol, 7,4′-Dihydroxyflavan, luteoforol, leukoefdin, afzelechin, Fisetinidol, Apiforol, Dihydrokaempferide, leukoefdin, Laricitrin, 5,7,4′-Tri-O-methylcatechin, (?)-Epicatechin quione, 4H-1-Benzopyran-4-one, 5,7 ,8-trihydroxy-2-(3,4,5-trihydroxyphenyl)-, 3′-Hydroxy-4′-O-methylglabridin, Mesquitol, Tricetinidin, (+)-Epiaromadendrin, L-Epicatechin, 1,2-Benzenediol, 4-(3,4-dihydro-7-hydroxy-2H-1-benzopyran-2-yl)-, Pinomyricetin, Epidistenin, 4′-O-methyepicatechin, Hibiscetin, Epimesquitol-4beta-ol, 4H-1-Benzopyran-4-one, 5,7-dihydroxy-2-(4-hydroxyphenyl)-3-methyl-, 4H-1-Benzopyran-4-one, 5-hydroxy-2-(3,4,5-trihydroxyphenyl)-, 2,3-Dihydrogossypetin, 2-(4-hydroxyphenyl)-3,4-dihydro-2h-chromene-4,5,7-triol, epi-Catechol, (2S)-dihydrotricetin, Taxifolin 3-O-acetate, Arachidoside, Leuco-fisetinidin, 3,5,7-trihydroxy-2-(3,4,5-trihydroxyphenyl)-3,4-dihydro-2H-1-benzopyran-4-one, “Isoetin, Guibourtinidol, 4′-O-Methylcatechin, Epicatechin 5,3′-dimethyl ether, 3-O-Methylepicatechin, Keto-teracacidin, Apigeniflavan, 3,5,8,3′,4′,5′-Hexahydroxyflavone.

Also disclosed are methods for identifying modulators of RAD52 biological activity, preferably for identifying inhibitors of RAD52 biological activity. The methods typically include contacting RAD52 with a compound and determining if the compound binds RAD52 and/or inhibits binding of RAD52 to ssDNA and/or inhibits RAD52 annealing of ssDNA, thereby identifying the inhibitor of RAD52 biological activity. In the disclosed methods, the RAD52 protein may be oligomerized such the ssDNA may be labeled at each end with one member of a FRET pair such that when the RAD52 protein binds the ssDNA in a reaction mixture, the FRET pair are brought into proximity for FRET to occur. Typically the ssDNA has a length of approximately 30 nucleotides. In the reaction mixture, the oligomerized RAD52 protein and the ssDNA may be present in approximate stoichiometric equivalence.

The compositions disclosed herein may be formulated as pharmaceutical composition for administration to a subject in need thereof. Such compositions can be formulated and/or administered in dosages and by techniques well known to those skilled in the medical arts taking into consideration such factors as the age, sex, weight, and condition of the particular patient, and the route of administration.

### EXAMPLES

The following examples are illustrative and should not be interpreted to limit the scope of the claimed subject matter.

### Example 1

### Small-Molecule Inhibitors Identify the RAD52-ssDNA Interaction as Critical for Recovery from Replication Stress and for Survival of BRCA2 Deficient Cells

Abstract

The DNA repair protein RAD52 is an emerging therapeutic target of high importance for BRCA-deficient tumors. Depletion of RAD52 is synthetically lethal with defects in tumor suppressors BRCA1, BRCA2 and PALB2. RAD52 also participates in recovery of the stalled replication forks. Anticipating that ssDNA binding activity underlies the RAD52 cellular functions, we carried out a high throughput screening campaign to identify compounds that disrupt the RAD52-ssDNA interaction. Lead compounds were confirmed as RAD52 inhibitors in biochemical assays. Computational analysis predicted that these compounds bind within the ssDNA-binding groove of the RAD52 oligomeric ring. The nature of the ligand-RAD52 complex was validated through an in silica screening campaign, culminating in the discovery of an additional RAD52 inhibitor. Cellular studies with our inhibitors showed that the RAD52-ssDNA. interaction enables its function at stalled replication forks, and that the inhibition of RAD52-ssDNA binding acts additively with BRCA2 or MUS81 depletion in cell killing.

Introduction

Understanding of synthetically lethal relationships between genome caretaker proteins will help to define the molecular mechanisms underlying the maintenance of genomic integrity and may lead to the advancement of personalized cancer treatments. Depletion of the human DNA repair protein RAD52 is synthetically lethal with defects in tumor suppressors, BRCA1, BRCA2, or PALB2 (Feng et al., 2011, Lok et al., 2012, Cramer-Morales et al., 2013). Importantly, this synthetic lethality requires both copies of the tumor suppressor gene to be defective and should not manifest in the heterozygous cells. Therefore, specific RAD52 inhibitors are expected to selectively kill cancerous cells lacking one of these three tumor suppressors. Replacing or supplementing standard radiation and chemotherapies with the RAD52 inhibitors will help to decrease the toxicity associated with these treatments.

BRCA1 and BRCA2 are tumor suppressors that are commonly mutated or depleted in hereditary and sporadic breast cancers, and have important roles in homologous recombination (HR) (Prakash et al., 2015), a template directed pathway that accurately repairs DNA lesions affecting both strands of the DNA duplex (Couedel et al., 2004, Moynahan and Jasin, 2010, Jasin and Rothstein, 2013, Kowalczykowski, 2015, Heyer, 2015b). BRCA1 regulates repair pathway choice after DNA damage by promoting HR (Kass and Jasin, 2010, Prakash et al., 2015). BRCA2 is a recombination mediator, which facilitates assembly of the RAD51 nucleoprotein filament on ssDNA downstream of BRCA1 activities (Couedel et al., 2004, Jensen et al., 2010, Liu et al., 2010, Thorslund et al., 2010, Prakash et al., 2015). PALB2 mediates interaction between BRCA1 and BRCA2 proteins, acts as a scaffold connecting numerous tumor suppressors (Park et al., 2014), stimulates Polmdependent DNA synthesis (Buisson et al., 2014) and RAD51 recombinase activity (Dray et al., 2010). Finally, the three tumor suppressors cooperate with Fanconi Anemia proteins in the repair of inter-strand DNA cross-links (Kim and D'Andrea, 2012).

Rad52 was identified in yeast as the main recombination mediator and the central player in the single-strand annealing pathway of mutagenic homology-directed DNA repair (Mortensen et al., 2009). In contrast to the severe recombination and repair phenotypes observed in yeast, deletion of RAD52 has only a mild effect on recombination in vertebrates (Rijkers et al., 1998, Yamaguchi-Iwai et al., 1998, Yanez and Porter, 2002). Although it is clear that RAD52 is important for survival and uncontrolled proliferation of BRCA-deficient cancer cells, the molecular mechanism by which RAD52 allows BRCA-deficient cells to survive is unknown. The proposed mechanisms included the putative RAD52 recombination mediator function and its role in single-strand annealing pathway of homology-directed DSB repair (Lok and Powell, 2012). Functional interactions between BRCA1, BRCA2, PALB2 and RAD52, as well as the ability of RAD52 to promote BRCA-independent cell survival, are commonly expected to involve the HR related mechanisms. The recent discovery that BRCA proteins act together with the Fanconi Anemia pathway to support and protect replication forks points to a potentially more complex scenario (Schlacher et al., 2011, Schlacher et al., 2012). Additionally, RAD52 cooperates with the structure-selective nuclease MUS81/EME1 to generate DNA double-strand breaks (DSBs) essential for the recovery of stalled replication forks in the absence of the replication check point (Murfuni et al., 2013).

Known biochemical functions of human RAD52 include annealing of two complementary ssDNA strands in the presence of replication protein A (RPA) (Van Dyck et al., 2001, Grimme et al., 2010) and the ability to pair ssDNA to complementary homologous regions in supercoiled DNA (Kagawa et al., 2001, Murfuni et al., 2013). Putative recombination mediator activity of RAD52 (Benson et al., 1998) should also require ssDNA binding. Therefore, if the cellular functions of the RAD52 protein depend on the ssDNA binding, then inhibition of the RAD52-ssDNA interaction should have similar consequences as RAD52 depletion. RAD52 forms an oligomeric ring (Kagawa et al., 2002, Lloyd et al., 2002, Singleton et al., 2002a, Stasiak et al., 2000), where the primary ssDNA binding site is located in the narrow groove spanning the ring circumference (Lloyd et al., 2005, Mortensen et al., 2002). We designated this ssDNA-binding groove as the feature to be targeted by small molecule inhibitors. While disrupting the protein-ssDNA interaction with small molecules presents a formidable challenge (Yap et al., 2012) that has only been overcome in a handful of cases, the ssDNA binding groove of RAD52 (for reasons discussed below) is a promising target and is distinct from the ssDNA binding sites of other ssDNA binding proteins.

Here we report development of a novel FRET-based high throughput screening (HTS) assay that led to the identification of compounds that disrupt the RAD52-ssDNA interaction. Initial HTS hits were biochemically validated in RAD52 functional assays and tested in two separate cellular assays. Two available high resolution crystal structures (PDB: 1H2I and 1KNO) of the conserved ssDNA-binding domain of RAD52 highlight the unique nature of this target (Singleton et al., 2002b, Kagawa et al., 2002). The ssDNA-binding region is continuous around the circumference of the ring and has shallow sub-pockets that are repeating in each monomer. While the truncated version of RAD52 in the crystal structures may differ from the full length RAD52, it likely recapitulates the structural features of the ssDNA-binding groove. Computational docking followed by all-atom simulated annealing placed all identified RAD52 inhibitors into two distinct sub-pockets within the ssDNA-binding groove. Compounds ‘1’ ((−)-Epigallocatechin) and ‘6’ (Epigallocatechin-3-monogallate) predicted to bind within the RAD52 ssDNA-binding site, inhibited formation of the RAD52-dependent DSBs in hydroxyurea (HU)-stressed, checkpoint deficient cells to the same level as RAD52 depletion. Moreover, ‘1’ acts additively with the MUS81 depletion to kill cells treated with hydroxyurea (HU), which perturbs replication, and with checkpoint inhibitor UCN01. These data strongly suggest that the ssDNA binding activity of RAD52 is required for recovery of stalled replication forks in checkpoint deficient cells. We also show that ‘1’ selectively kills cells depleted of BRCA2, further supporting the importance of the RAD52-ssDNA interaction in BRCA deficient cells and the potential therapeutic value of RAD52 inhibition. Finally, in order to validate the strength of our hypotheses about the structural nature of the ligand-RAD52 complex, we developed a validated in silico screening campaign, based on our HTS results, using a library of four thousand natural products. We describe the discovery of NP-004255, a macrocyclic compound, which we show by NMR WaterLOGSY and biophysical assays to be a completely novel and effective inhibitor of the RAD52-ssDNA interaction. The implication of these findings for the discovery of novel therapeutics that specifically inhibit the activity of RAD52 is discussed.

Results

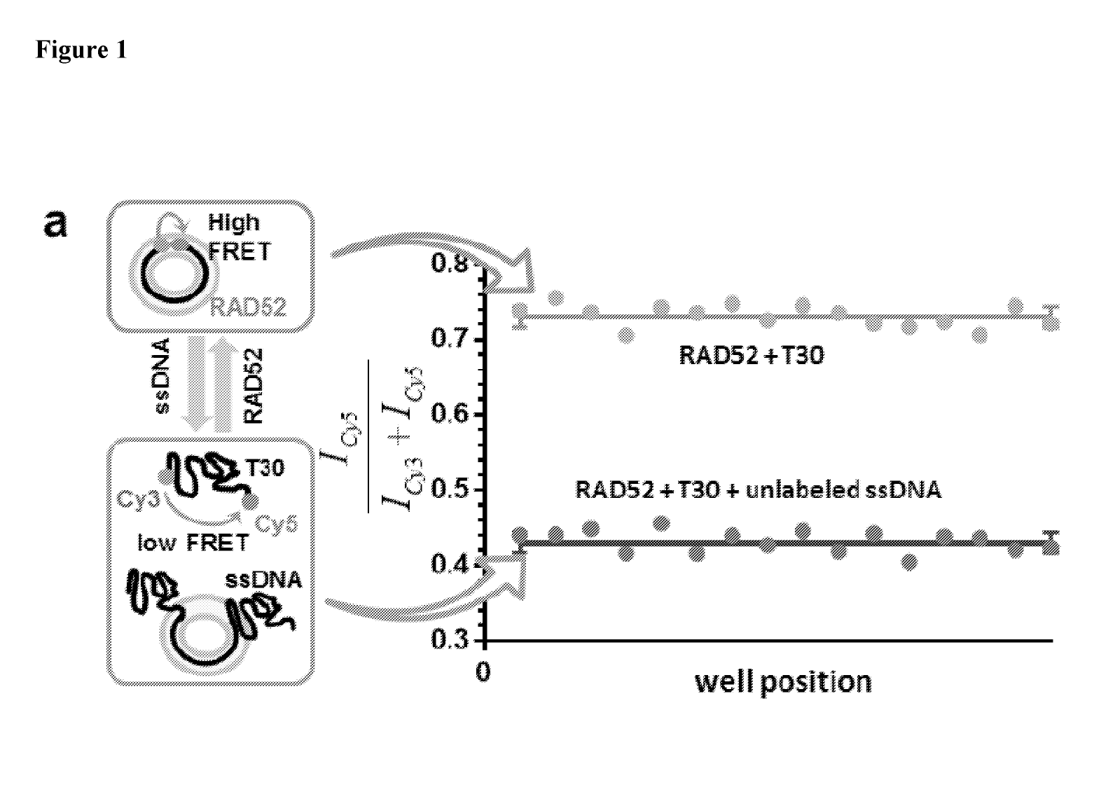

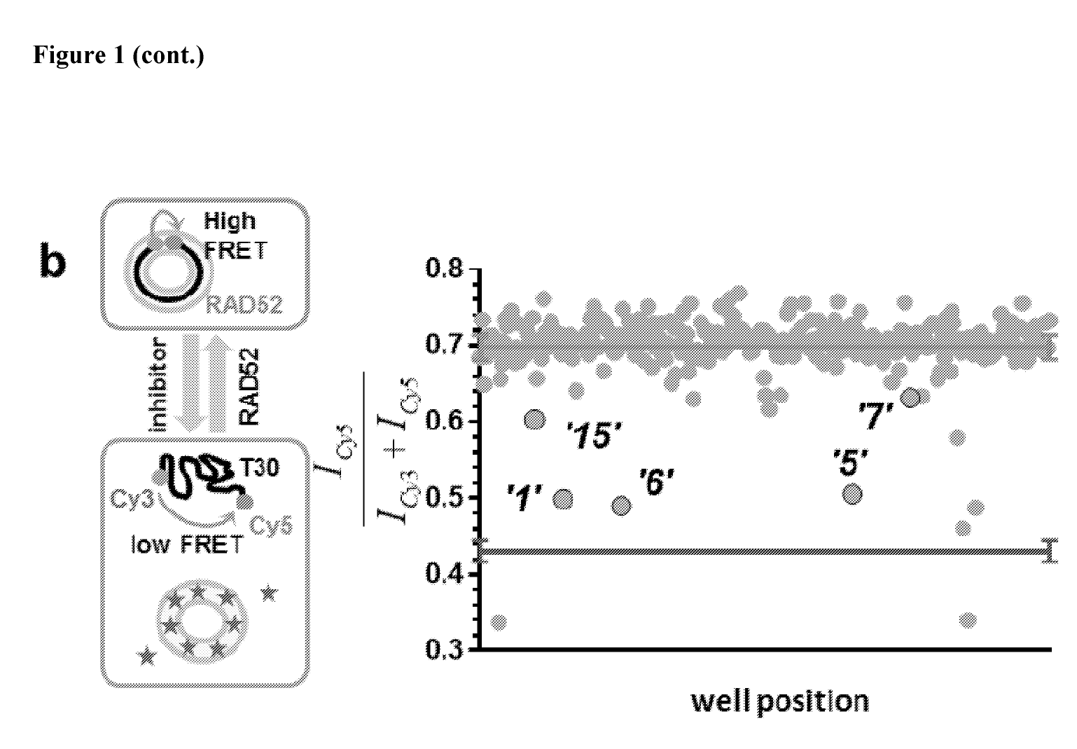

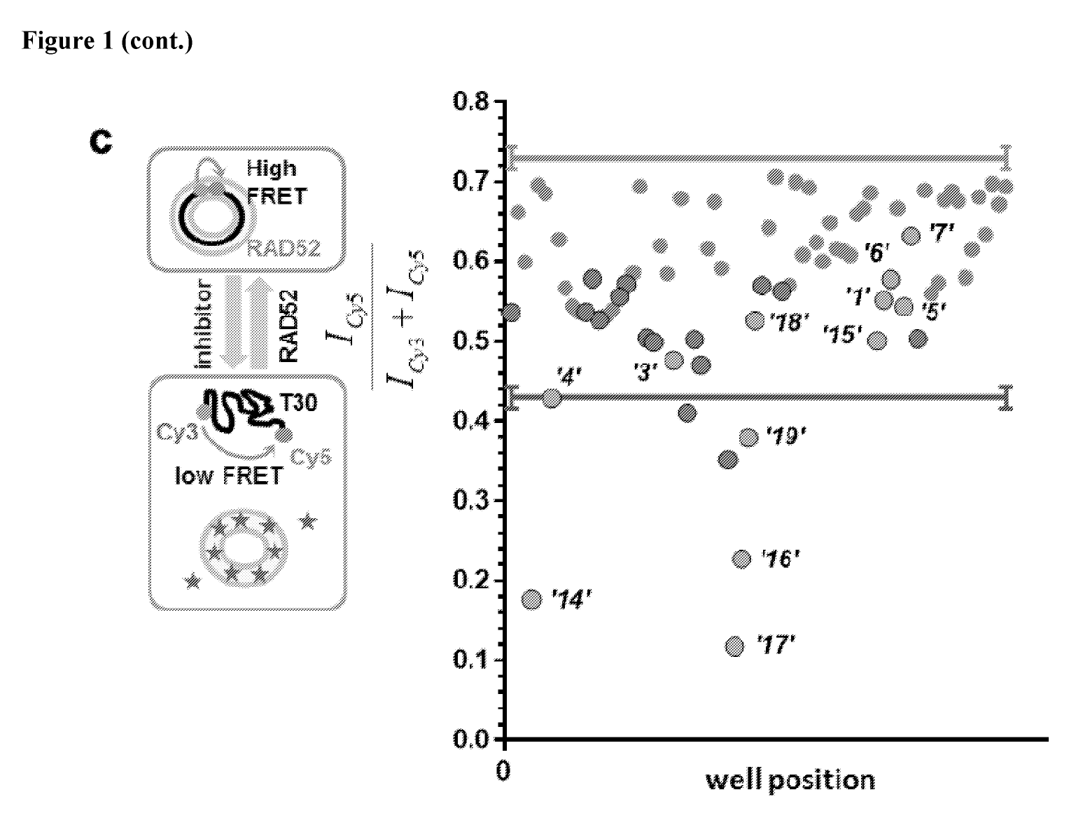

High Throughput Screening (HTS) of the MicroSource SPECTRUM collection identifies compounds that inhibit the RAD52-ssDNA interaction: To identify compounds that disrupt the RAD52-ssDNA interaction we adapted a previously developed FRET-based assay (Grimme and Spies, 2011, Grimme et al., 2010) to the HTS format. The RAD52-ssDNA interaction is independent of sequence and involves a binding site size of 4 nucleotides per monomer (Singleton et al., 2002a). Our FRET-based assay relies on the ability of RAD52 to bind and wrap ssDNA around the narrow groove spanning the circumference of the protein ring (Grimme and Spies, 2011, Grimme et al., 2010). A Förster Resonance Energy Transfer (FRET) donor (Cy3) and acceptor (Cy5) fluorophores are positioned at the ends of a 30-mer ssDNA (Cy3-dT₃₀-Cy5). When this ssDNA forms a stoichiometric complex with RAD52 (one 30-mer ssDNA molecule per one heptameric ring of RAD52), the two fluorophores are brought close to one another resulting in an increase in the FRET signal. The assay was successfully adapted to the 384-well plates HTS format. In each well, we recorded the fluorescence signal of the Cy3 dye, which was excited directly, and the signal of Cy5 dye, which was excited via the energy transfer from Cy3. The apparent FRET signal was then calculated as described in the Materials and Methods. The separation between the positive control (a stoichiometric complex of RAD52 with Cy3-dT₃₀-Cy5 substrate challenged with excess of unlabeled ssDNA (Poly dT100)) and the negative control (an unperturbed stoichiometric complex of RAD52 with Cy3-dT₃₀-Cy5) initially resulted in a Z′ factor of 0.66 when calculated for the whole plate. Further optimization increased the Z′ factor calculated for the control rows in the screening experiments to 0.94, indicating excellent reliability of the assay (FIG. 1a). Using this assay, we screened the MicroSource SPECTRUM collection, which contains 2,320 drug and drug-like synthetic compounds as well as natural products, which represent a wide structural diversity and a range of known biological activities. The screening was carried out at 15 μM concentration of each compound in the library. Of the 2,320 compounds examined, 96 were identified as preliminary hits. The results for a one plate in the collection are shown in FIG. 1b with initial hits that were validated in the follow up experiments highlighted in green. These preliminary hits were selected based on the criterion of their separation from the negative control (RAD52+Cy3-dT₃₀-Cy5) of at least 5 S.D. The 96 preliminary hits were assembled into a “cherry picked plate” and were tested in two more rounds of screening FIG. 1c. Compounds that showed reproducible and nearly complete inhibition were re-tested at a range of small-molecule concentrations. Seven of the compounds tested showed a promising decrease in FRET in the re-screening assays and six were selected for biochemical validation (shown in green). One compound was excluded due to a low molecular weight and promiscuous binding observed in the follow-up biochemical assays. Additionally, we selected six compounds that elicited the FRET values below the positive control. These molecules were expected either to be “false positives” (i.e. molecules that are fluorescent in the Cy3 channel or interact with DNA) or to have a significant absorbance in the region of Cy3 emission and/or Cy5 excitation. We purchased 12 compounds and confirmed their chemical structures by 1D NMR. The identified compounds and their chemical structures are listed in the Table 1.

TABLE 1
The twelve hits from the FRET-based HTS assay aimed at finding inhibitors of the RAD52-ssDNA interaction.

| | Small | | IC₅₀ | | |
|---|---|---|---|---|---|
| | Molecule | | (DNA binding); | IC₅₀ | SAEM |
| | Name; | | FRET value | Annealing | ΔG |
| # | CAS # | Small Molecule Structure | at saturation | Extent) | (kcal/mol) |
| ‘1’ | (−)-Epigallo- catechin; 970-74-1 | 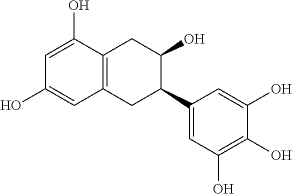 [SMILES: Oc1cc(O)c2c(c1)C[C@H](c1cc(O)c(O)c(O)c1)[C@H](O)C2] | ssDNA: 1.8 ± 0.1 μM; 0.45 ± 0.004 ssDNA-RPA: 1.6 ± 0.1 μM; | ssDNA: 4.9 ± 0.4 μM ssDNA-RPA 4.8 ± 1.8 μM; | −8.60 |
| ‘3’ | Methacycline Hydrochloride; 3963-95-9 | 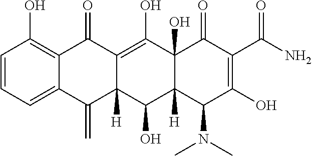 [SMILES: C=C1c2cccc(O)c2C(=O)C2=C(O)[C@]3(O)C(=O)C(C(N)=O)=C(O)[C@@H](N(C)C)[C@@H]3[C@@H](O)[C@H]12] | 2.0 ± 0.17 μM; 0.47 ± 0.01 | 3.8 ± 0.2 μM | −4.61 |
| ‘4’ | Rolitetra- cycline; 751-97-3 | 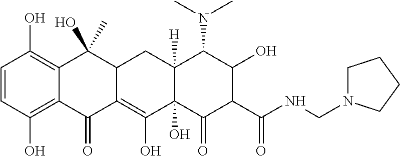 [SMILES: CN(C)[C@@H]1C(O)C(C(=O)NCN2CCCC2)C(=O)[C@@]2(O)C(O)=C3C(=O)c4c(O)ccc(O)c4[C@@](C)(O)C3C[C@@H]12] | 29 ± 8.2 μM; 0.56 ± 0.04 | NI | −10.5 |
| ‘5’ | (−)-Epicatechin gallate; 1257-08-5 | 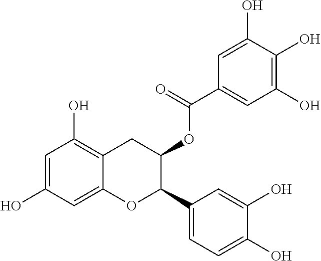 [SMILES: O=C(O[C@@H]1Cc2c(O)cc(O)cc2O[C@@H]1c1ccc(O)c(O)c1)c1cc(O)c(O)c(O)c1] | 255 ± 16 nM; 0.41 ± 0.004 | 20 ± 0.7 μM | −9.87 |
| ‘6’ | Epigallo- catechin-3- monogallate; 989-51-5 | 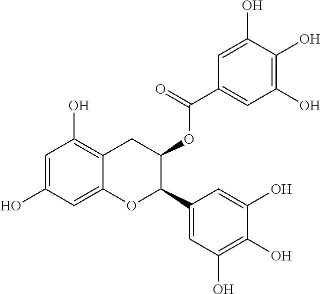 [SMILES: O=C(O[C@@H]1Cc2c(O)cc(O)cc2O[C@@H]1c1cc(O)c(O)c(O)c1)c1cc(O)c(O)c(O)c1] | ssDNA: 277 ± 22 nM; 0.46 ± 0.01 ssDNA-RPA: 1.6 ± 0.5 μM; | ssDNA: 6.7 ± 2.1 μM ssDNA-RPA: 3.7 ± 0.5 μM; | −10.69 |
| ‘7’ | (−)-Epicatechin; 490-46-0 | 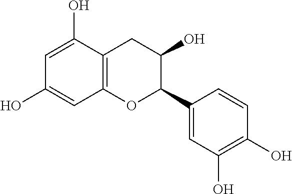 [SMILES: Oc1cc(O)c2c(c1)O[C@H](c1ccc(O)c(O)c1)[C@H](O)C2] | 1.45 ± 0.11 μM; 0.51 ± 0.01 | NI | −9.03 |
| ‘14’ | Oxidopamine; 28094-15-7 1199-18-4 | 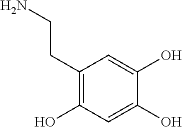 [SMILES: NCCc1cc(O)c(O)cc1O] | 779 ± 51 nM; 0.50 ± 0.01 | NI | −5.71 |
| ‘15’ | Quinalizarin; 81-61-8 | 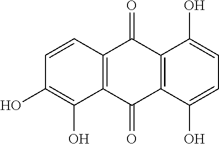 [SMILES: O=C1c2ccc(O)c(O)c2C(=O)c2c(O)ccc(O)c21] | 563 ± 40 nM; 0.51 ± 0.01 | 5.6 ± 0.6 μM | −9.17 |
| ‘16’ | Cisapride Monohydrate; 260779-88-2 81098-60-4 | 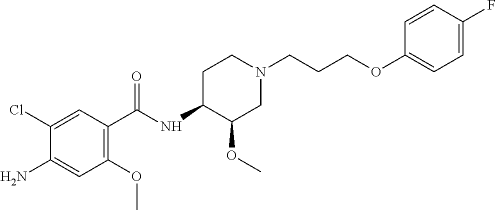 [SMILES: COc1cc(N)c(Cl)cc1C(=O)N[C@H]1CCN(CCCOc2ccc(F)cc2)C[C@H]1OC] | 1.06 ± 0.05 μM; 0.50 ± 0.01 | NI | −8.39 |
| ‘17’ | Cedrelone; 1254-85-9 | 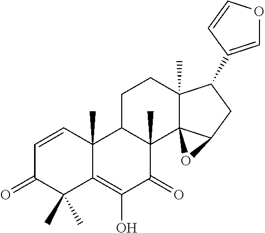 | >300 μM | NI | −10.0 |
| ‘18’ | Asiatic Acid; 464-92-6 18449-41-7 | 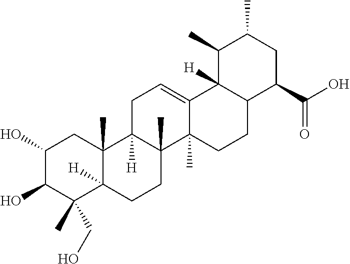 | >800 μM | >100 μM | −11.33 |
| ‘19’ | Gossypetin; 489-35-0 | 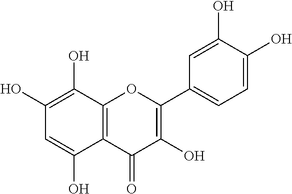 [SMILES: O=c1c(O)c(-c2ccc(O)c(O)c2)oc2c(O)c(O)cc(O)c12] | 913 ± 58 nM; 0.49 ± 0.01 | 6.0 ± 2.3 μM | −9.30 |

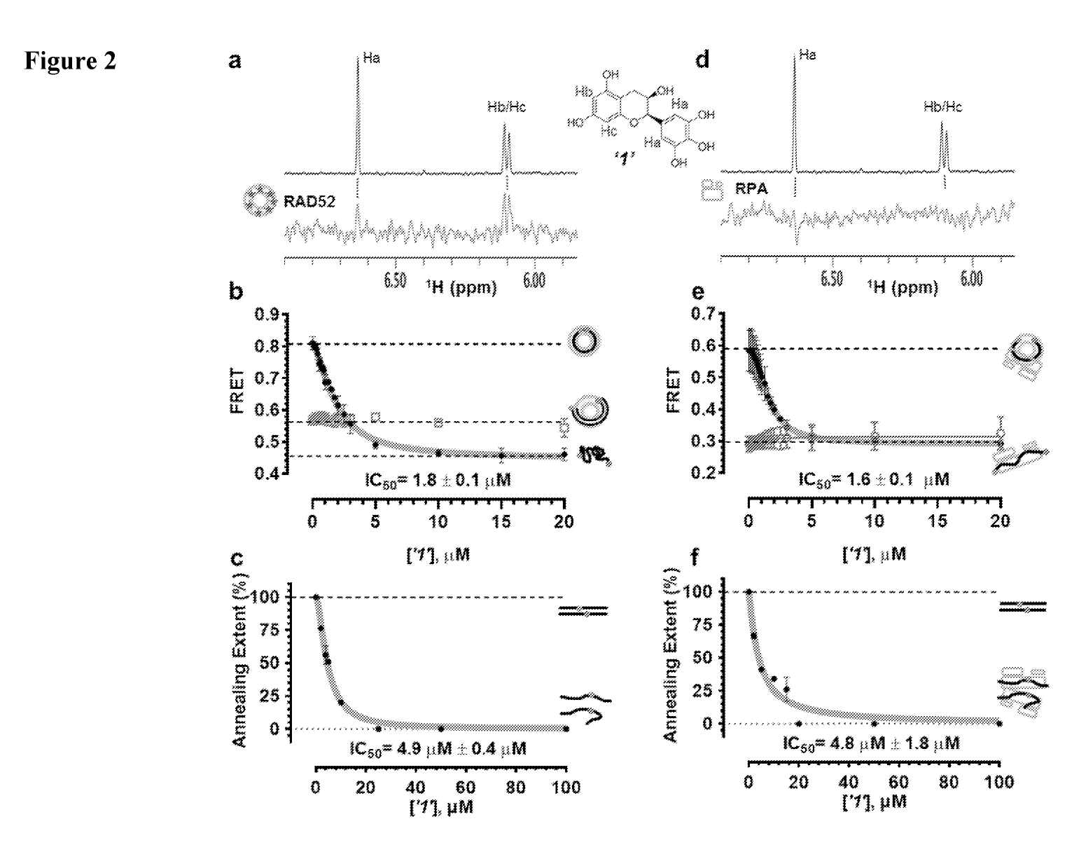

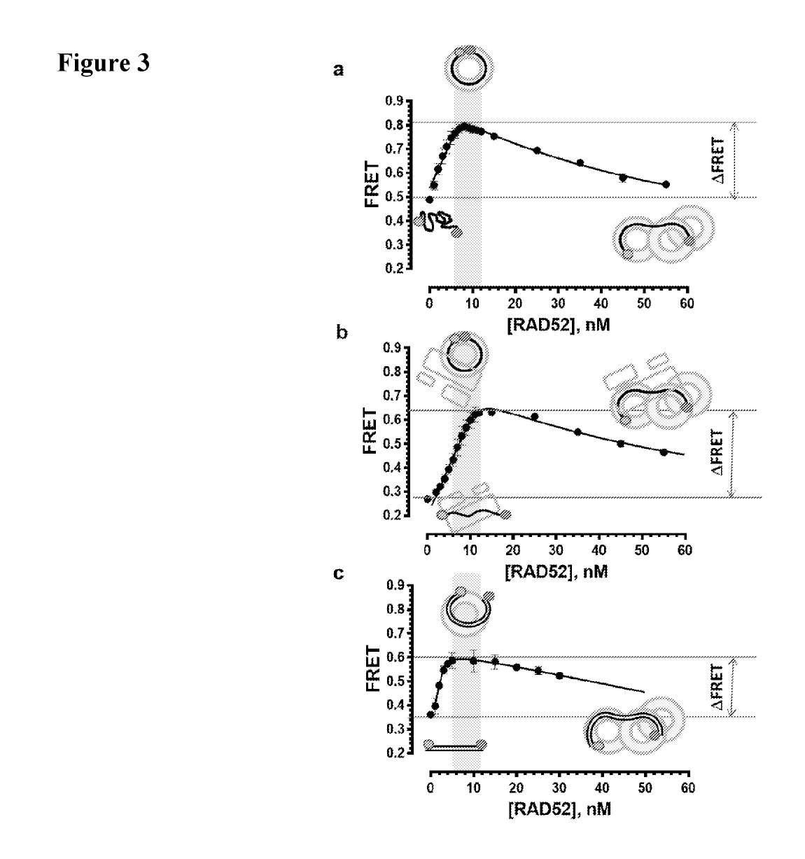

Selected compounds inhibit ssDNA binding and wrapping by RAD52 with IC₅₀ values ranging from mid-nanomolar to high-micromolar range: In order to determine how the selected compounds affect known RAD52 functions we performed FRET-based assays that recapitulate the HTS screen, yet have higher precision and yield a calibrated FRET signal. We titrated increasing amounts of each compound into a cuvette containing preformed stoichiometric RAD52-Cy3-dT₃₀-Cy5 complexes (1 nM Cy3-dT₃₀-Cy5 and 8 nM RAD52). As the compounds bind and disrupt the ssDNA-RAD52 interaction we observed a decrease in FRET between the DNA-tethered Cy3 and Cy5 fluorophores. At each concentration of the compound, the FRET signal was adjusted for the change in Cy3 and Cy5 fluorescence in the presence of the compound, but in the absence of protein. From the hyperbolic inhibition curves we then calculated IC₅₀ value for each compound under these conditions (FIGS. 2b and 3b, Table 1). IC₅₀ values were in the nano-molar range for compounds ‘5’, ‘6’, ‘14’, ‘15’ and ‘19’. We calculated IC₅₀ values in the micro-molar range for compounds ‘1’, ‘3’, ‘7’, ‘13’, and ‘16’. Compound ‘4’ had IC₅₀ value in the mid micro-molar range. Compounds ‘17’ and ‘18’ were poor inhibitors of ssDNA binding with IC₅₀ values in the high micro-molar range. These compounds were likely false positives in our HTS screen.

Selection of the compounds that inhibit RAD52-mediated ssDNA annealing: To determine how the selected compounds affect the ssDNA annealing function of RAD52 we performed FRET-based annealing assays (Grimme et al., 2010, Grimme and Spies, 2011). These assays utilize two complementary single stranded 28-nucleotide-long substrates, which contain either Cy3 (T-28) or Cy5 (P-28) incorporated into the middle of the respective DNA strand. When the substrates are annealed by RAD52, the Cy3 and Cy5 dyes are separated by 3 base pairs and yield a high FRET signal (FIG. 2 Supplement 2). Negative controls containing T-28 and P-28 with the compounds in the absence of RAD52 displayed no change in FRET suggesting the small molecules do not do not promote ssDNA annealing by themselves. The annealing reactions were initiated by mixing two half reactions and observing the change in FRET over time in the presence of varying concentrations of small molecules. An increasing FRET value over time indicates formation of the dsDNA duplex which brings the two dyes in close proximity (FIG. 2 Supplement 2). Fitting the annealing data to a double exponential allowed us to calculate and compare the final extent of annealing at varying concentrations of each compound compared to RAD52 alone. We plotted the final extent of annealing vs the concentration of the compound and calculated an IC₅₀ of annealing inhibition. A full set of the annealing time courses recorded at different concentrations of ‘1’ is shown in FIG. 2 Supplement 2. As the concentration of the compound increases, the final extent of annealing is reduced compared to RAD52 alone. Since we showed previously that ssDNA wrapping around the RAD52 ring is necessary for the most efficient annealing (Grimme et al., 2010, Honda et al., 2011), it was expected that the compound that blocks access of the ssDNA to the ssDNA binding groove of RAD52 will compete with DNA annealing, thus shifting the equilibrium away from the dsDNA product. It is notable that the IC₅₀ values for DNA annealing were generally higher than IC₅₀ values for the ssDNA binding. We attribute this to the dynamic nature of the RAD52-ssDNA complex as well as to RAD52 ability to bypass regions of heterology and other obstacles during the homology search process (Rothenberg et al., 2008). To confirm specificity of the two compounds (‘1’ and ‘6’) selected for the in-depth follow-up characterization as disruptors of the RAD52-ssDNA interaction, we tested the ability of these compounds to interfere with the RAD52-dsDNA interaction, which involves a different site on the RAD52 ring (Kagawa et al., 2008, Grimme et al., 2010). At the stoichiometric RAD52: dsDNA ratio, (1 nM dsDNA: 10 nM RAD52) the dsDNA is bent upon RAD52 binding, which allows us to distinguish the RAD52-bound and free dsDNA (FIG. 2 Supplement 1). Interestingly, ‘1’ had no effect on the RAD52-dsDNA interaction (FIG. 2b open grey squares), which indirectly confirms its specificity for the ssDNA-binding groove of RAD52. In contrast, ‘6’ was able to displace dsDNA from the RAD52-dsDNA complex (FIG. 3b open grey squares). Dynamic light scattering experiments conducted in the presence of equimolar concentrations of each compound and RAD52 showed that the presence of these compounds neither breaks up the oligomeric ring of RAD52 nor causes protein aggregation (FIG. 2 Supplement 3). Notably, this means that our compounds act differently from the RAD52 inhibitor 6-hydroxy-DL-dopa (Chandramouly et al., 2015), which disrupts supramolecular assembly of the RAD52 protein. We further confirmed that the inhibition of the ssDNA binding does not occur due to aggregation of compounds as annealing FRET trajectories in the presence of 0.01% Triton X-100 are identical to those in the absence of Triton X-100.

Compounds ‘1’ and ‘6’ physically interact with RAD52: To confirm that the selected compounds bind RAD52, we employed water-ligand observation with gradient spectroscopy (WaterLOGSY), an NMR technique, which is based on transfer of magnetization from bulk water to the protein-bound compound of interest (Dalvit et al., 2001, Dalvit et al., 2000). In WaterLOGSY spectrum, if a compound binds to a protein, the compound will receive negative nuclear Overhauser effects (NOEs) due to the slow tumbling of the protein-compound complex, leading to a positive WaterLOGSY peak. In contrast, if a compound does not bind to a protein, the compound will receive positive NOEs due to the fast tumbling of the compound itself, resulting in a negative WaterLOGSY peak. FIG. 2a and FIG. 3a show that both ‘1’ and ‘6’ physically interact with RAD52 protein. FIG. 2a shows the aromatic region of the 1D ¹H NMR spectrum of compound ‘1’ alone (black) and the WaterLOGSY spectrum of 20 μM compound ‘1’ in the presence of 3.3 μM RAD52 (red). Clearly, positive WaterLOGSY peaks are observed for the compound ‘1’, indicating the binding of ‘1’ to RAD52. Similarly, FIG. 3a depicts the aromatic region of the 1D ¹H NMR spectrum of compound ‘6’ alone (black) and the WaterLOGSY spectrum of 40 μM compound ‘6’ in the presence of 3.3 μM RAD52 (red). Again, positive WaterLOGSY peaks are clearly detected for the compound ‘6’, thus confirming the binding of ‘6’ to RAD52. Notably, ‘6’ also binds to RPA as shown by the positive WaterLOGSY peaks (FIG. 3d), thought it does not interfere with the RPA-ssDNA interaction (FIG. 3e), while ‘1’ neither binds to RPA as shown by the negative WaterLOGSY peak (FIG. 2d) nor interferes with the RPA-ssDNA interaction (FIG. 2e).

Compounds ‘1’ and ‘6’ inhibit RAD52 binding to and annealing of the RPA-coated ssDNA: In the cell, ssDNA is typically found in complex with Replication protein A (RPA), which is the major eukaryotic ssDNA-binding protein essential for DNA replication, repair and recombination (Wold, 1997, Oakley and Patrick, 2010, Chen and Wold, 2014). The RPA-ssDNA complex is a natural substrate for the RDA52-mediated strand annealing. To confirm that compounds ‘1’ and ‘6’ can inhibit the RAD52 binding to and annealing of the RPA-coated ssDNA we added stoichiometric amounts of RPA (1 RPA per 30 nucleotides of ssDNA) to the FRET-based ssDNA binding/wrapping and ssDNA annealing experiments described above. RPA binds ssDNA with high affinity and extends the ssDNA to its contour length. In our assays such an extension manifests as a distinct FRET state of ˜0.3, which is readily distinguished from a FRET state of ˜0.48 of free Cy3-dT₃₀-Cy5 ssDNA, as well as ˜0.63 FRET of the stoichiometric ssDNA-RPA-RAD52 complex (see FIG. 2 Supplement 1 and (Grimme and Spies, 2011) for details). Notably, neither compound ‘1’ nor compound ‘6’ affected the RPA-ssDNA interaction over the range of the tested compound concentrations (FIG. 2e and FIG. 3e). Both, however, inhibited the RAD52 binding to and wrapping of RPA-coated ssDNA with IC₅₀ values identical to those determined without RPA (FIG. 2e and FIG. 3e). Similarly, we confirmed that both compounds ‘1’ and ‘6’ inhibit the RAD52-mediated annealing of RPA-coated ssDNA with the IC₅₀ values comparable to the inhibition of ssDNA annealing (FIG. 2f and FIG. 3f). Notably, this inhibition is not due to the disruption of RAD52-RPA interaction as neither ‘1’ nor ‘6’ interfered with the interaction between the two proteins (FIG. 2 Supplement 4).

Virtual screening places the RAD52 inhibitors within the ssDNA binding groove: In order to gain insight into the binding determinants of the various polyphenol hits obtained from the HTS screening, we undertook a computational investigation using the structure of the oligomeric ring formed by the conserved ssDNA-binding domain of RAD52 (PDB 1KNO). We utilized a layered approach involving docking and all atom simulated annealing with explicit solvent, using a knowledge based force field (Krieger et al., 2004). The long circular ssDNA binding groove of RAD52 oligomeric ring yielded excellent “druggability” scores (˜4.0), based on the pocket metric of Sugo et al., (Soga et al., 2007). Docking approaches generally generate many potential poses, and many false positives. Initially, top scoring poses in either Triangle Matcher (placement), London dG (affinity scoring function) or MM/GBSA (physics based scoring) were retained for further analysis (see Methods section for details).

An all atom force field-based protocol was employed to distinguish binding affinities from a variety of docking poses that possessed various docking metrics. We compared the different scoring metrics, such as Triangle Matcher placement scores followed by rescoring with the affinity function versus the computationally expensive force field-based ligand refinement and subsequent MM/GBSA scoring. The use of all atom simulations (including explicit solvent models), when combined with docking, has been shown to significantly boost docking procedures' ability to predict and rank compound affinities. Therefore, we can compare the differences in free energy changes due to ligand binding for each of the poses, an ability that is not all within the realm of classical docking procedures (Ellingson et al., 2015, Whalen et al., 2011, Warren et al., 2006, Head, 2010). Thirty four unique docking poses were selected for comparison for each compound. Comparisons of the results of the various scoring metrics for docking of ligands to the RAD52 complex showed a clear lack of consensus between the three methods, except for compound ‘6’, which resulted in a single pose scoring the highest in all three methods. To determine which methodology consistently provides the most accurate scoring, we employed a conservative approach, in which the 34 selected top scoring complexes were further subjected to all atom simulated annealing studies, using explicit solvent and salt conditions.

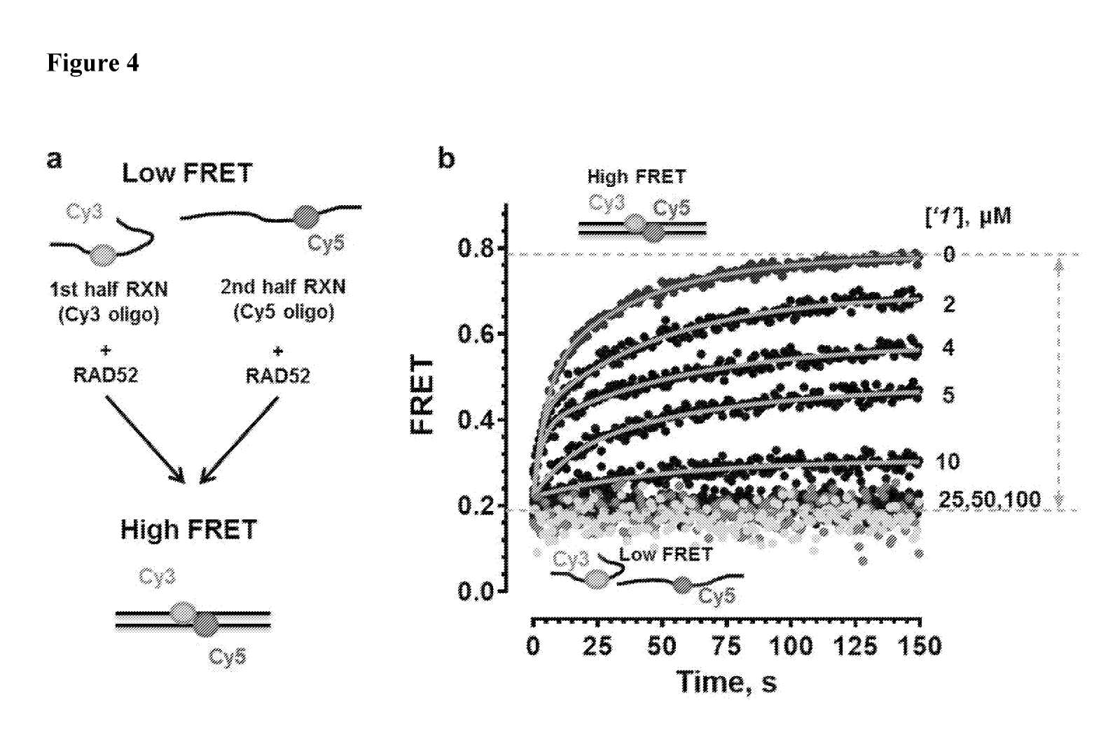

The binding affinities computationally determined using the Simulated Annealing Energy Minimization (SAEM) Docking approach are listed in the Table 1). Interestingly, in most cases, the RAD52-ligand complex resulting from the best placed docking pose, rather than the more computationally intensive MM/GBSA (physics-based) scoring function, yielded the final SAEM-generated RAD52-ligand complex with the lowest energy. Compounds ‘1’ and ‘6’ yielded complexes with unique binding sub-pockets or “hotspots” along the RAD52 binding groove, suggesting that they may have distinct biological activities and/or efficacies with regard to their ability to compete with ssDNA binding (FIG. 4). The SAEM-generated complexes indicate that compounds ‘1’ and ‘6’ occupy complex pockets lying at the interface of two RAD52 monomers. Notably, all final compound placements include interactions, directly or through the interstitial water molecules, with key RAD52 residues, which have been previously shown to be involved in ssDNA binding (Lloyd et al., 2005) (FIG. 4). In particular, R55, Y65, K152, R153 and R156 found in the vicinity of the docked compounds (FIG. 4b) have been shown to impact ssDNA binding (Lloyd et al., 2005). Additional participants in the binding of our inhibitors include K141 and K144 residues that are important to distinct cellular functions of yeast Rad52. A highly conserved K144 corresponds to K159 in S. cerevisiae Rad52. Its K159A substitution results in severe deficiency in mitotic recombination, mild γ-ray sensitivity, but unperturbed recombination between direct repeats (Mortensen et al., 2002). K141 corresponds to S. cerevisiae R156, whose substitution to alanine causes γ-ray sensitivity only (Mortensen et al., 2002).

All of the tested compounds yielded complexes in which interstitial solvent plays a role in the binding of the ligand. Unlike classic enzyme pockets, which often have large desolvated volumes, the RAD52 ssDNA binding groove cannot truly be evaluated for the ability to bind to compounds without understanding the role of solvent in its various sub-pockets. The SAEM method used here was specifically designed to capture these complex recognition parameters. Unlike the case of deeply buried waters that occur in many active site pockets of enzymes, the waters along the RAD52 ssDNA-binding groove are mostly not involved in productive interstitial H-bonding with the ligand, but rather, represent a van der Waals binding surface, suggesting opportunities for future ligand improvement.

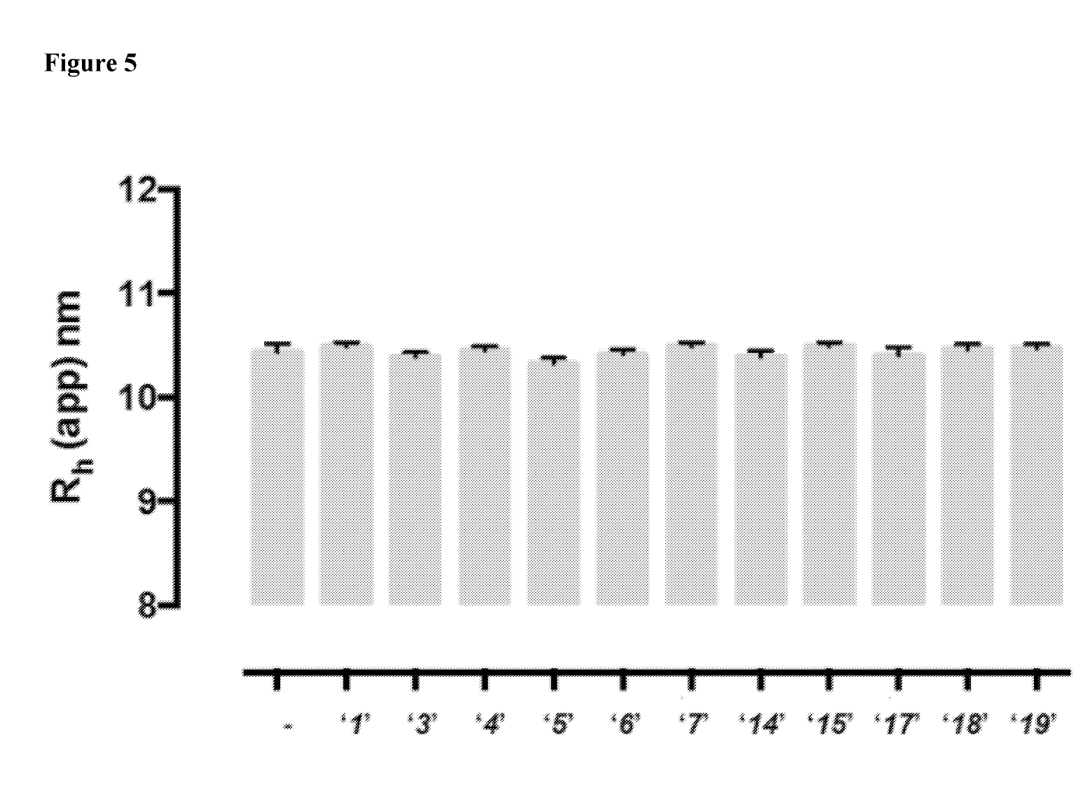

Inhibiting the RAD52-ssDNA interaction interferes with RAD52/MUS81-mediated DSB formation essential for the replication fork recovery in check point deficient cells: In human cells, RAD52 may perform both limited recombination-mediator function in the RAD51-dependent pathway (Lok and Powell, 2012, Benson et al., 1998, Feng et al., 2011) as well as additional RAD51-independent roles (Lok and Powell, 2012, Murfuni et al., 2013, Mcllwraith and West, 2008). One of these HR independent roles of RAD52 involves stimulation of MUS81/EME1-dependent DSB formation at the replication forks stalled by hydroxyurea (HU) treatment in the absence of cellular checkpoints (Murfuni et al., 2013). Since the most likely targets of these MUS81/EME1/RAD52-dependent DSBs are the DNA structures produced by RAD52 (Murfuni et al., 2013), we expected this activity to depend on the RAD52-ssDNA interaction (FIG. 5a). To confirm this, we assessed DSB formation in the checkpoint deficient cells using the neutral comet assay. These assays monitor MUS81/EME1/RAD52-dependent DSB formation upon induction of replication stress by HU treatment in primary fibroblasts immortalized by hTERT expression and treated with UNC01 to inhibit CHK1 kinase (FIG. 5b). Our data show that increasing amounts of ‘1’ and ‘6’ decrease the mean tail moment indicative of the decrease in the MUS81/EME1/RAD52-dependent DSB formation (FIG. 5). Importantly, these inhibitors recapitulate RAD52 depletion by inhibiting RAD52-MUS81-dependent DSBs at stalled replication forks (FIG. 5a-b). Notably, even at 500 nM, ‘1’ had the same effect of reduction in DSBs as siRNA depletion of RAD52. These data strongly support the idea that inhibiting the RAD52-ssDNA interaction in cells recapitulates the effects of RAD52 depletion with respect to its role at the distressed replication forks. It also confirms our previous supposition that the target of MUS81/EME1-mediated cleavage under these conditions are indeed the structures annealed by RAD52. Interestingly, the concentrations of ‘1’ sufficient to inhibit the MUS81/EME1/RAD52-mediated DSBs correlate well with the IC₅₀ values for inhibition of ssDNA binding/wrapping in vitro (compare FIG. 5c with FIGS. 2b, and e). These values are significantly lower than those required for inhibiting annealing of short, complementary oligonucleotides (FIGS. 2c and f). Higher concentration of ‘6’ required to inhibit MUS81/EME1/RAD52-mediated DSBs (FIG. 5d) is likely due to the particular chemical nature of this compound, which is a promiscuous binder; not only does it interact with RPA and binds within the dsDNA binding site of RAD52, but has been identified as an inhibitor in 192 different HTS assays (Pubchem). Treatment of RAD52-depleted cells with a dose of “6” that is sufficient to reduce DSBs as efficiently as 1 μM of “1” consistently failed to further decrease the formation of DNA breaks (FIG. 5b). We previously reported that concomitant depletion of RAD52 and MUS81 gives raise to the MUS81-independent DSBs (Murfuni et al., 2013). In agreement with a specific activity towards RAD52, treatment of the MUS81-depleted cells with “1” resulted in an appearance of the MUS81-independent DSBs upon replication stress induced by CHK1 inhibition. Due to its lower capacity to inhibit the MUS81/EME1/RAD52-mediated DSBs, and its expected off-target effects, we have eliminated ‘6’ from further analysis and focused all our subsequent cellular studies on ‘1’, appeared more specific in our biochemical studies and had no Pubchem hits. The fact that none of the compounds we tested showed additive effects on DSBs with RAD52 siRNA depletion, suggests that the effect of these inhibitors is specific to RAD52 at least with respect to recovery from replication stress. Furthermore, accumulation of anaphase bridges, a phenotype associated with impairment of the RAD52-independent mitotic function of MUS81/EME1 was completely unaffected by inhibition of RAD52, whereas it was strongly stimulated by MUS81 silencing (FIG. 5 Supplement 1). This observation strongly suggests that the suppression of the DSB formation is not due to direct inhibition of MUS81.

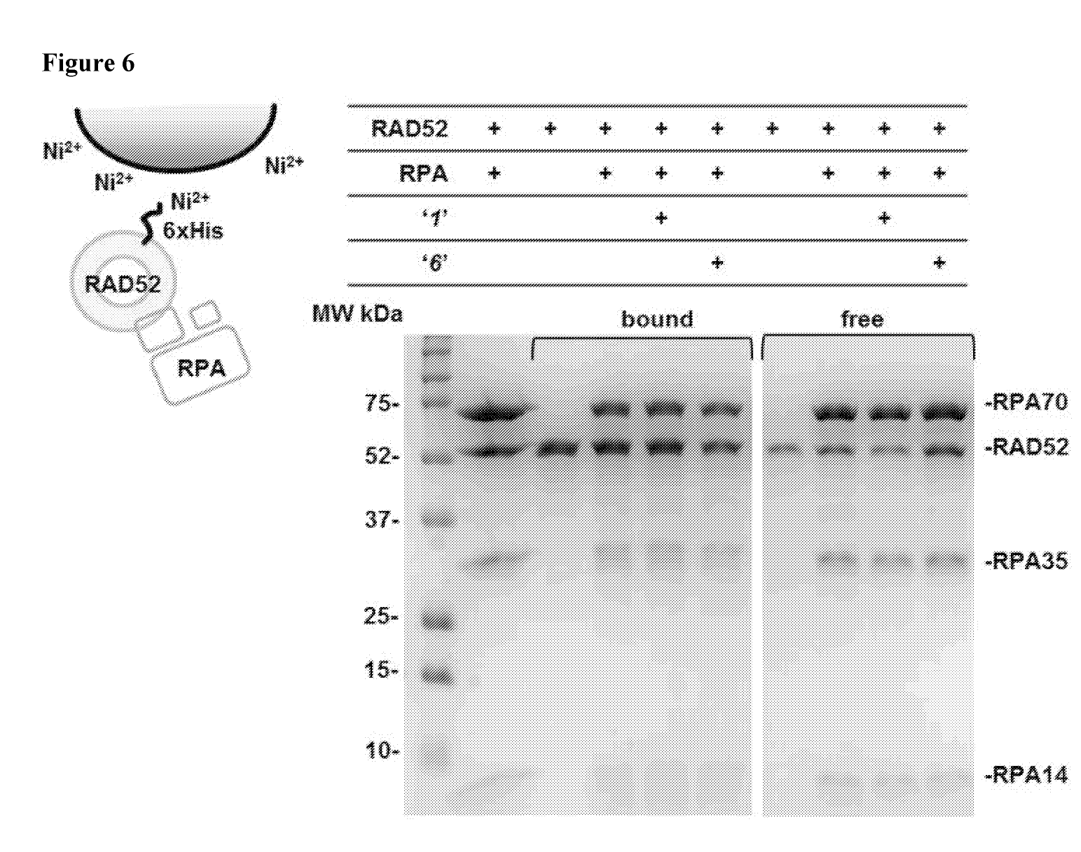

Inhibiting the RAD52-ssDNA interaction kills BRCA2-depleted cells, as well as MUS81-depleted cells under pathological replication conditions: Compounds ‘1’ and ‘6’ are able to interfere with MUS81-dependent DSBs formation under pathological replication, mimicking RAD52 depletion (FIG. 5). To address whether inhibition of the ssDNA-RAD52 binding reduces viability of MUS81-depleted cells, as reported for RAD52 depletion (Murfuni et al., 2013), we evaluated cell death after inducing replication stress by pharmacological CHK1 inhibition (FIG. 6). In cells transfected with the control (Ctrl) siRNAs, replication stress induced by HU treatment resulted in a 20% of cell death, which was increased similarly by MUS81 or BRCA2 knock-down by nearly 2-fold. Treatment with ‘1’ also potentiated the effect of the combined HU+UCN01 treatment and, strikingly enhanced cell death observed in MUS81-depleted cells. Interestingly, inhibition of RAD52 increased cell death of MUS81-depleted cells even under unperturbed cell growth to approximately the same level as RAD52 depletion by siRNA (Murfuni et al., 2013). Strikingly, no additive effect on cell death was detected in cells depleted of RAD52 and treated with the RAD52 inhibitor, as compared with the cells transfected with the RAD52 siRNAs alone (FIG. 6b).

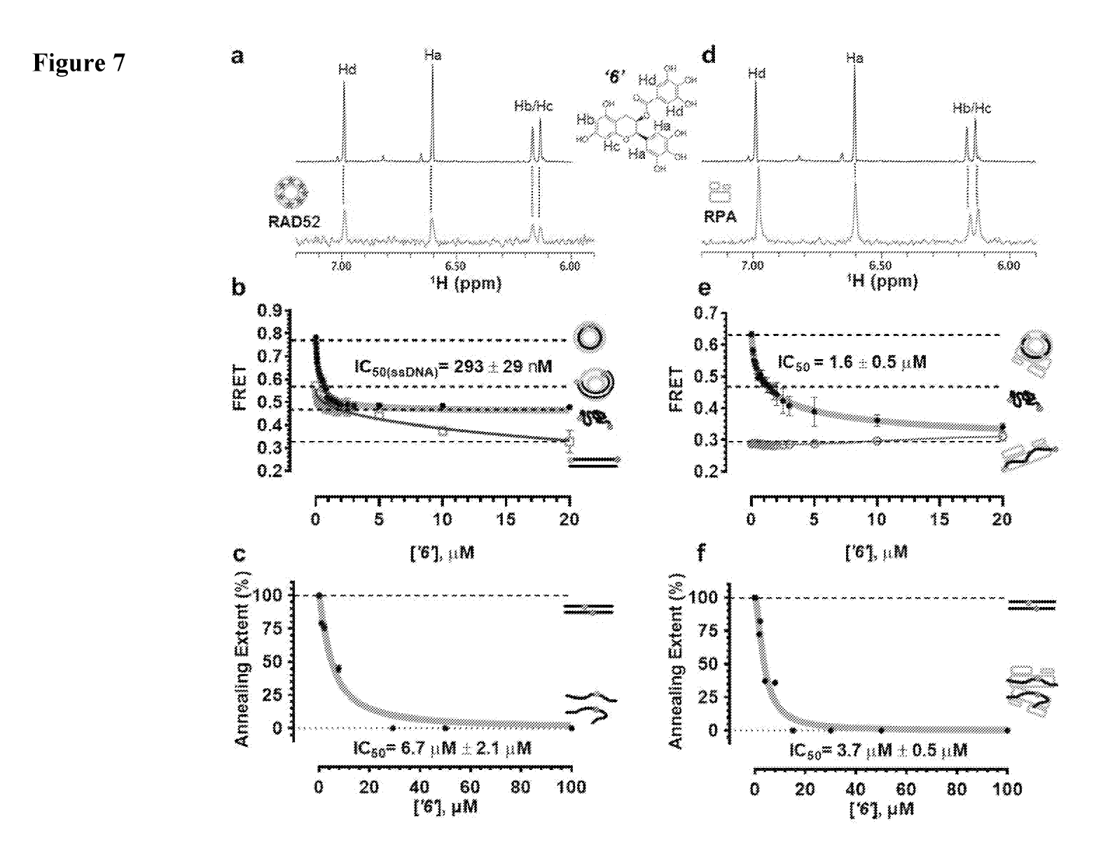

Depletion of RAD52 not only enhances cell death of MUS81-depleted cells, but also reduces viability of BRCA2-deficient cells, making RAD52 an attractive target for potential treatment of BRCA2-deficient tumors (Feng et al., 2011, Warren et al., 2006). Strikingly, treatment with ‘1’ acted additively with loss of BRCA2 resulting in ˜80% of cell death after replication stress (FIGS. 6b and c). Interestingly, concomitant depletion of BRCA2 and MUS81 also resulted in an additive effect on cell death, even under an unperturbed cell growth (FIGS. 6b and c). To further investigate the effect of ‘1’ on cell viability under the conditions of pathological replication, we depleted cells with BRCA2 or RAD52 siRNAs and challenged them with HU for 18 h in the presence or absence of ‘1’. As reported in FIG. 7, and in agreement with previous reports, co-depletion of BRCA2 and RAD52 increased cell death with respect to each single depletion. Interestingly, RAD52 inhibition mimicked RNAi-mediated gene knockdown and induced a substantial increase in cell death of BRCA2-depleted cells after prolonged replication arrest, further supporting the possibility that inhibiting the RAD52-ssDNA interaction may be a useful strategy for targeting the BRCA2-deficient tumor cells.

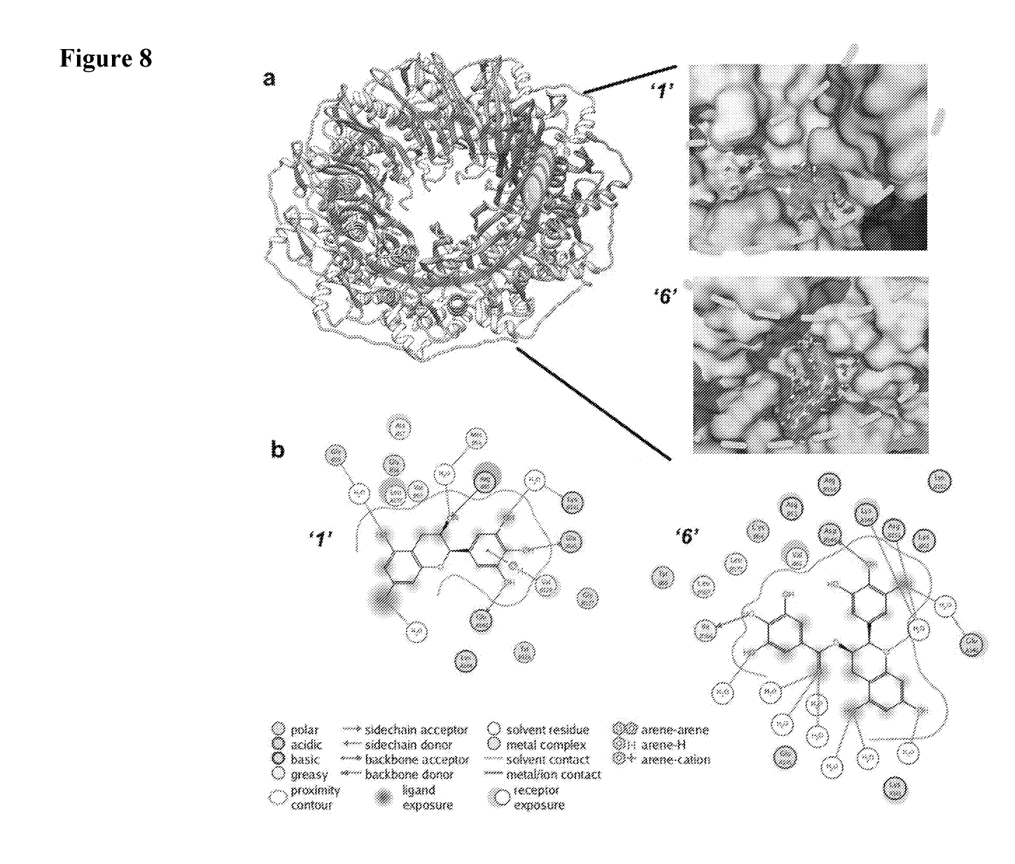

In Silico Screening and Discovery of NP-00425: translating structure-activity relationships from HTS into novel inhibitors: We hypothesized that the computationally determined RAD52-‘1’ and RAD52-‘6’ complexes could be used to validate an in silico screening workflow directed towards identifying a novel inhibitor of the RAD52-ssDNA interaction. This approach should facilitate further discovery of novel drug lead compounds that possess similar or improved activities as ‘1’ and ‘6’, but with fundamentally different chemical space. Natural products have an unrivaled history in drug discovery, and often represent the first and most significant hits against a metabolic pathway. The AnalytiCon Discovery MEGx Natural Products Screen Library, which is the in silico version of an actual library of purified natural products from plant, fungal and microbial sources, was subjected to an in silico screening campaign (FIG. 8a). The campaign was designed based on the ability to optimally minimize false positives and false negatives, and to maximize true positives and true negatives. More specifically, the experimental hits identified in the HTS campaign described above, constitute the true positives, while specifically selected decoy compounds constitute the true negatives. The details of this optimization approach, known as Receiver Operator Characteristic (ROC), are described in the Methods section. The ROC procedure in this in silico screening study was designed to challenge the value of the docking and scoring methods by employing decoy compounds (so called ‘DUDS’ compounds; see Methods section), which possess similar chemical properties, but different topologies than true positives. Importantly, the ROC approach uses the experimental HTS hits to optimize the in silico screening assay vis-à-vis minimizing false positives (which are rampant in all docking-based in silico screening approaches). The ROC curve for ‘1’ (FIG. 8b) shows that the optimized in silico selection process is nearly ideal in separating false positives from true positives. Finally, an in silico screen of the AnalytiCon Discovery MEGx Natural Products data base resulted in 9 compounds that had poses with scores better than those of compound ‘1’. The best scoring of those structures was ordered from AnalytiCon for in vitro inhibition studies. The compound that was identified, NP-004255 is known as corilagin, and is a member of the class of secondary plant metabolites called ellagitannins. Corilagin is a macrocylic ester consisting of three trihydroxylated phenolic moieties. NP-004255 binds to RAD52 in a similar manner as ‘1’ and ‘6’, in that it uses a buried interstitial water network, and is able to adopt a conformation that fits nicely into the ssDNA binding groove (FIGS. 8d and e). The binding and the inhibitor activity of this prediction was validated by both NMR and FRET-based assays, as described below.

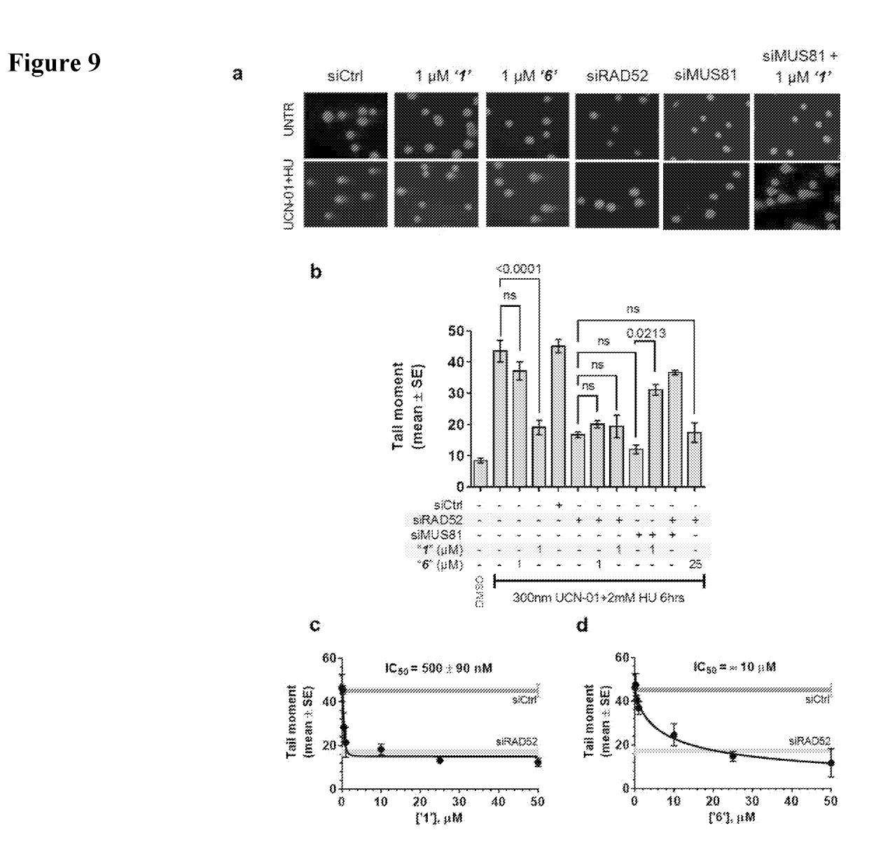

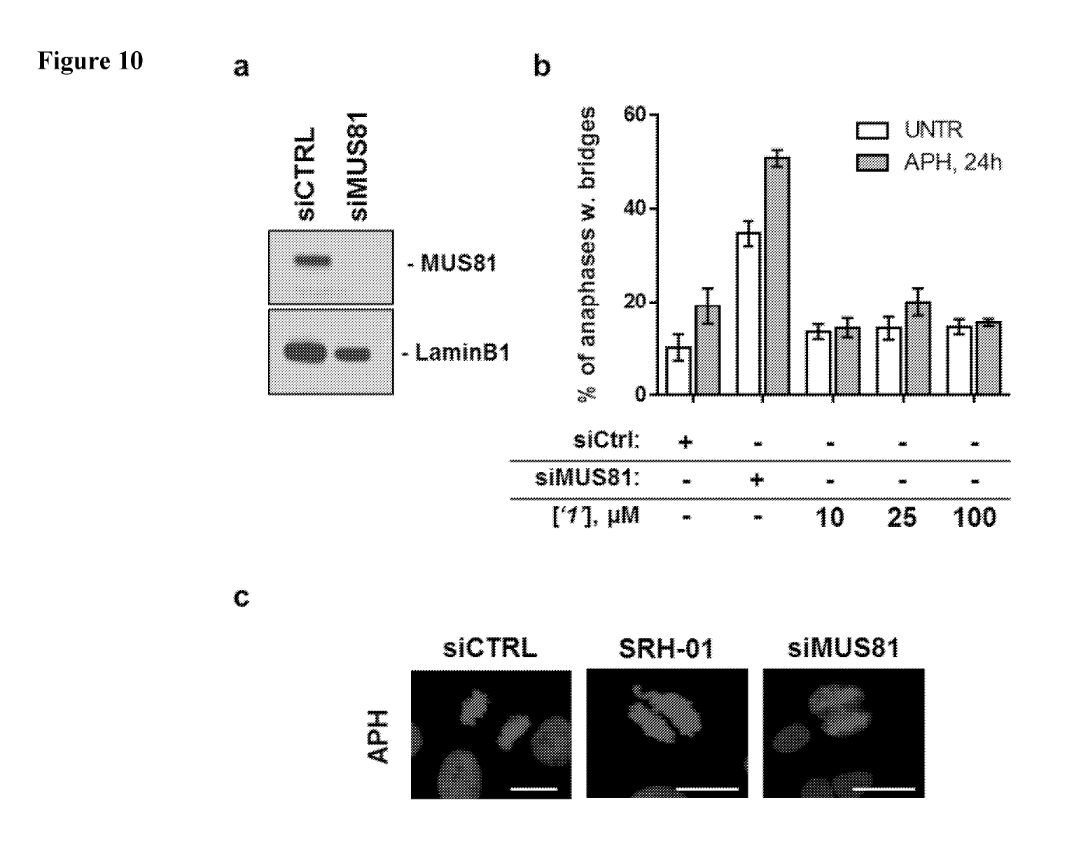

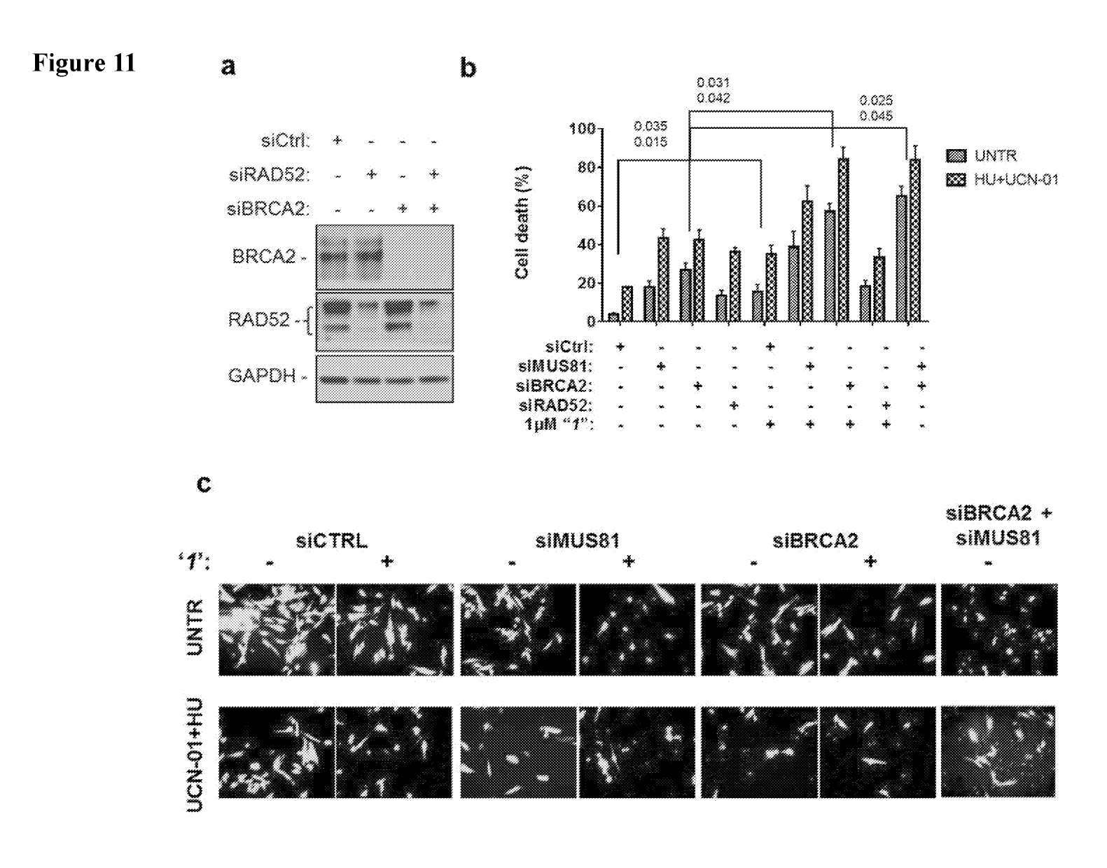

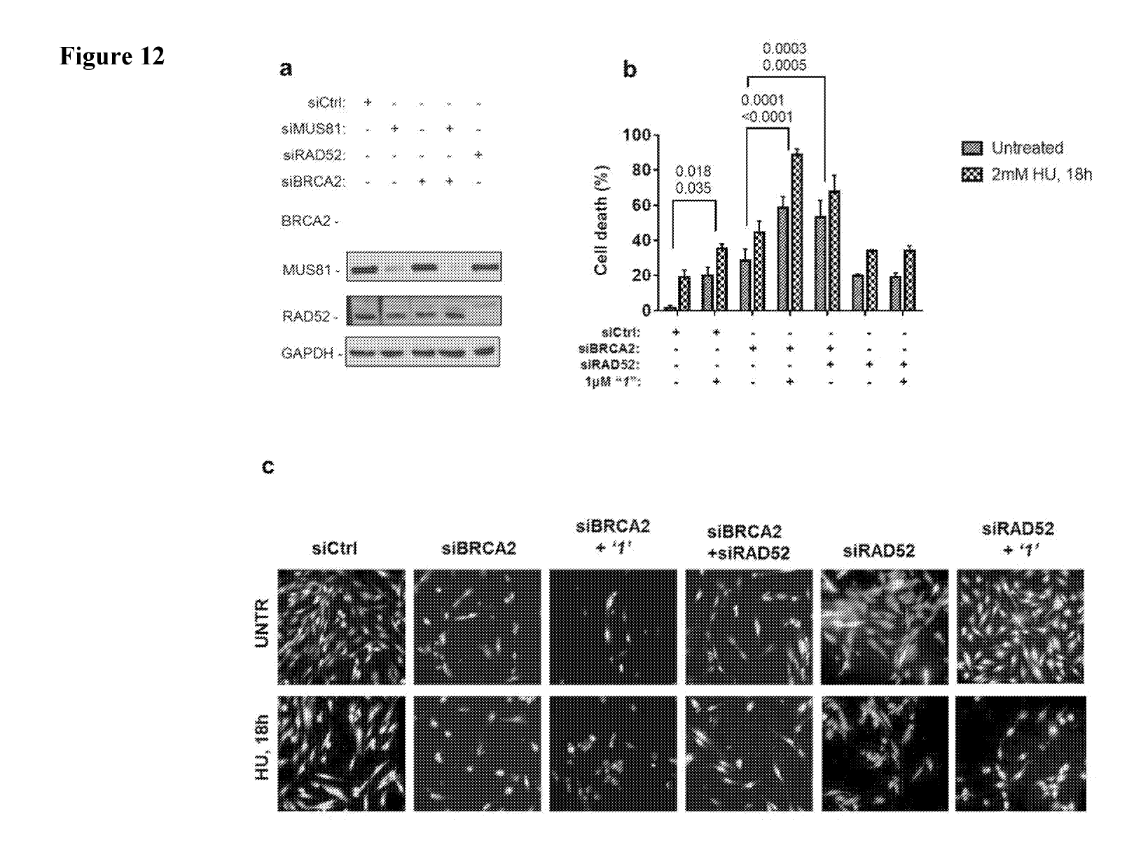

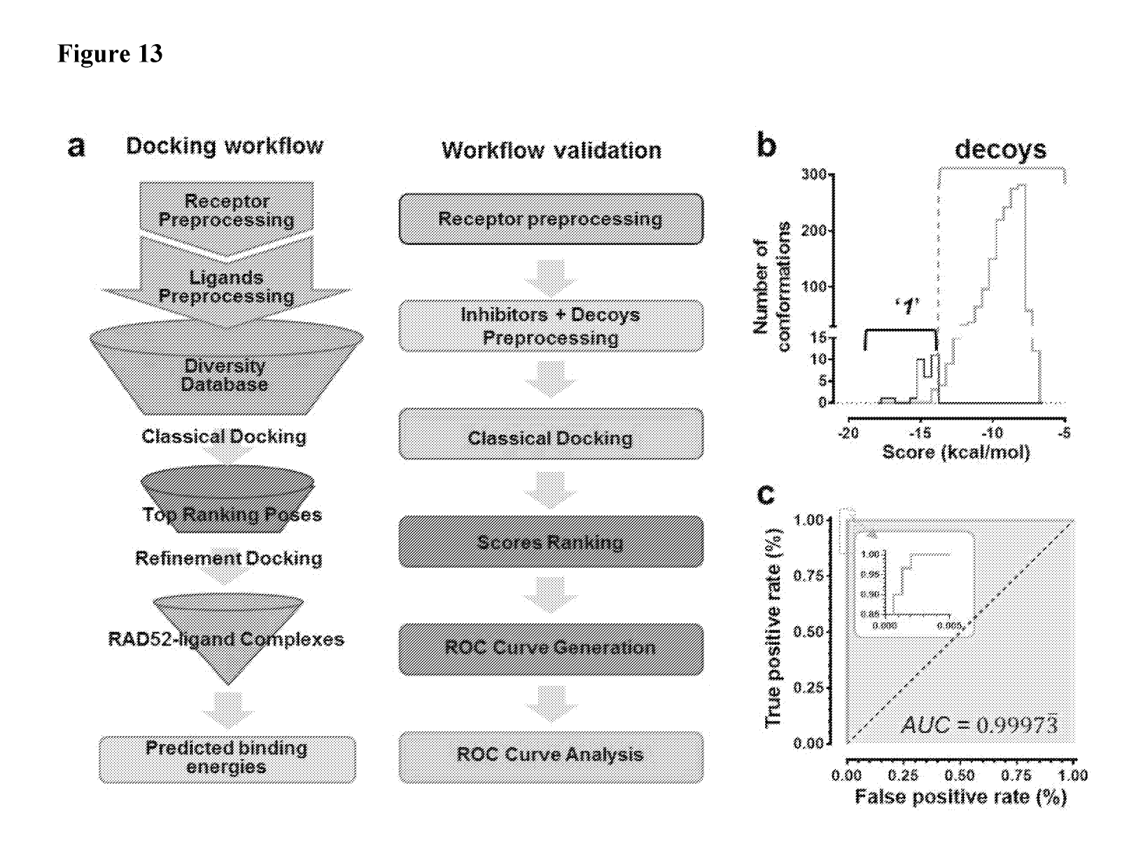

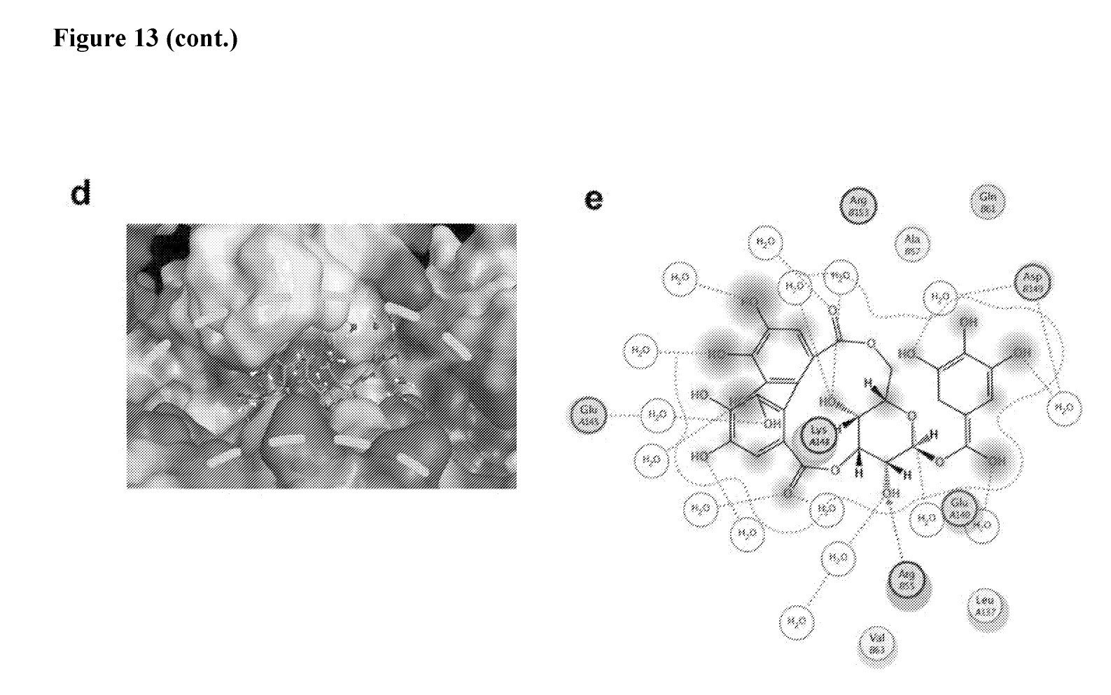

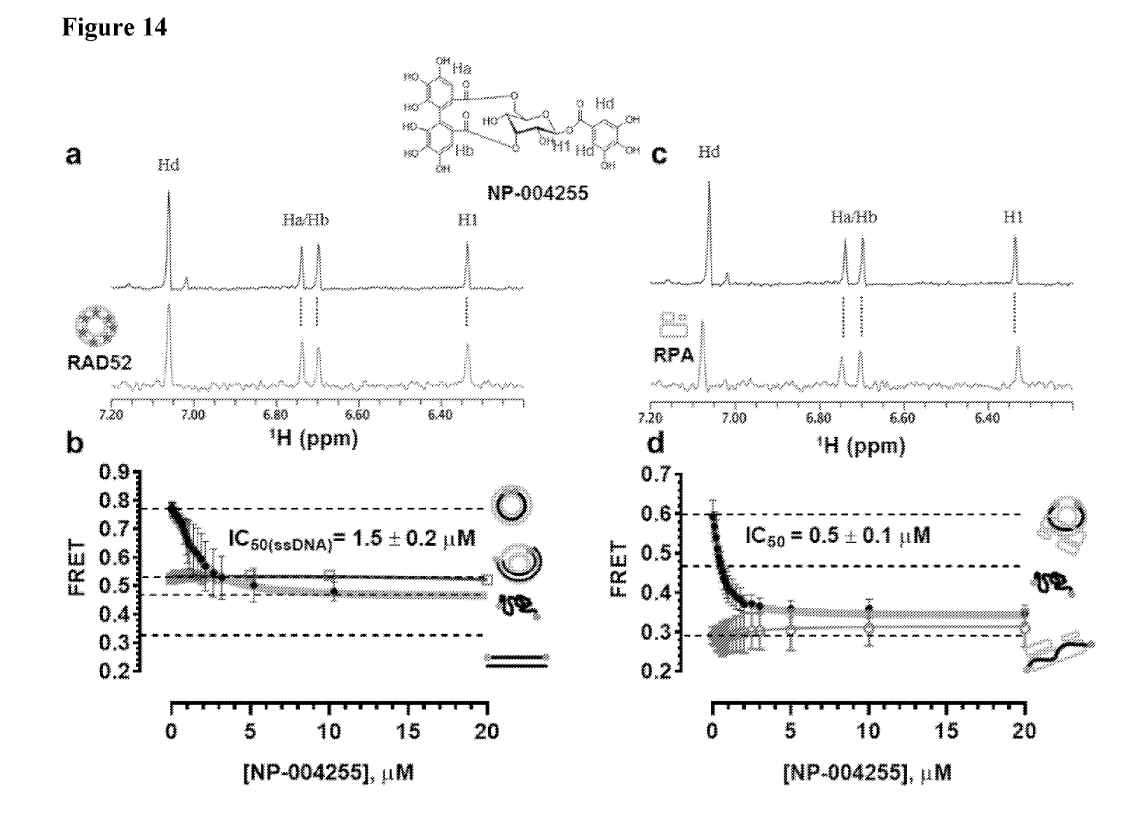

Natural product NP-00425 physically interacts with RAD52 and RPA proteins, inhibits the RAD52 binding to ssDNA and the ssDNA-RPA complex, but does not affect RAD52-dsDNA interaction or the ssDNA binding by RPA: The biochemical assays that were carried out for the compounds ‘1’ and ‘6’, were repeated for the NP-004255 assay (FIG. 9). The WaterLOGSY spectra suggest that similar to ‘1’ and ‘6’, NP-004255 physically interacts with RAD52 protein (FIG. 9a), while the FRET-based competition assay (FIG. 9b) confirmed that this natural product does indeed inhibit the RAD52-ssDNA interaction with the IC₅₀=1.5±0.2 μM, which is a potency similar to ‘1’. Also similar to ‘1’, the macrocycle compound was specific for the RAD52-ssDNA complex and had no effect on the RAD52-dsDNA interaction (FIG. 9b).

Similar to ‘6’, NP-004255 also bound RPA (FIG. 9c), but did not affect the RPA-ssDNA complex (FIG. 9d). It did, however, inhibit the RAD52 binding to the RPA-coated ssDNA with the IC₅₀=0.5±0.1 μM, i.e. it was more effective in perturbing this interaction that that involving the protein-free ssDNA.

Discussion

Maintenance of genetic integrity, as well as the ability to accurately and timely repair damaged DNA and complete DNA replication are essential for all living organisms (Heyer, 2015a, Abbas et al., 2013). While these basic processes and the central protein players are conserved, significant variation exists between eukaryotic lineages. The mechanisms that ensure faithful DNA replication and repair are exceedingly more complex in mammalian cells compared to simpler eukaryotes with more alternative interconnected pathways that may share proteins as well as the regulatory enzymes. HR and the pathways that employ the machinery of HR are expected to be responsible for the most accurate repair of the most deleterious DNA lesions including DSBs, DNA interstrand cross-links, and damaged replication forks (Head, 2010, Li and Heyer, 2008, Couedel et al., 2004, Moynahan and Jasin, 2010, Jasin and Rothstein, 2013).

In yeast, Rad52 functions as a recombination mediator by facilitating replacement of RPA with Rad51 recombinase on ssDNA and thereby allowing formation of the Rad51 nucleoprotein filament, which is the active species in the DNA strand exchange reaction. Analogously, RAD51 nucleoprotein filament formation and its activity in human cells is facilitated by a recombination mediator, BRCA2 (Xia et al., 2001, Yang et al., 2005, Carreira et al., 2009) with multiple RAD51 paralogs playing roles in ensuring assembly and stability of the active RAD51 nucleoprotein filament (Yang et al., 2005, Chun et al., 2013). Whether and how human RAD52 substitutes for BRCA2 mediator activity remains unclear. Synthetic lethality between BRCA2 defects and RAD52 depletion suggests that either RAD52 is indeed a recombination mediator, or that it participates in an alternative pathway that becomes prominent in the absence of BRCA2 function, such as for example SSA (single-strand annealing) (Feng et al., 2011, Lok and Powell, 2012). More intriguingly, RAD52 depletion is also synthetically lethal with defects in BRCA1, a tumor suppressor that acts upstream of BRCA2 in HR and at the branch point in the DSB repair that promotes homology-directed DNA repair through HR or SSA over the NHEJ (non-homologous end joining) (Singleton et al., 2002a). It is unknown which pathway(s) allow survival and proliferation of BRCA-deficient cells. These pathways, however, have to depend on the activities or interactions of RAD52. We showed recently that human RAD52 plays an important role in allowing cellular recovery under conditions of pathological replication (Murfuni et al., 2013). Similarly, a sub-pathway of HR that is Rad51 (Rhp51) independent, but Mus81/Eme1/Rad52 (Rad22) dependent has been described in yeast and represents an important mechanism of DNA repair during replication in fission yeast (Doe et al., 2004, Vejrup-Hansen et al., 2011). Whether this pathway, at least in part, compensates for the BRCA-deficiency in human cells remains to be determined.

We chose to target the well characterized ssDNA binding activity of RAD52, which we expected to underlie RAD52 functions both in supporting replication and in promoting the survival of BRCA-deficient cells. The ssDNA-binding groove of RAD52 is an interesting target for small-molecule binding in that it spans the circumference of the RAD52 oligomeric ring and offers a repetitive pattern of potential binding pockets. This deep and circular groove surprisingly yields reasonable druggability scores, as described in the Results section. Nevertheless, it is a highly exotic cavity, and very distinct from enzyme and receptor pockets. The ssDNA binding groove consists of an alternating arrangement of hydrophobic and hydrophilic regions. Twelve compounds that inhibit RAD52-ssDNA interaction identified in this study (Table 1) are predicted to bind within the ssDNA binding groove of RAD52 ring. In retrospect, it is not surprising that molecules such as the current suite of polyphenols have high affinity for this cavity. Additionally, although there are a number of hydrophobic regions in the ssDNA binding groove, one does not see the kind of significant desolvation that is usually found in enzyme active sites. Nevertheless, a number of waters are revealed in the crystal structure, and are maintained in the docking simulations. However, the nature and importance of these water networks to ligand optimization is not known. It appears there is significant room for improvement in terms of matching the shape of the binding groove with the van der Waals surface of prospective ligands. It will be interesting to see whether such chemical space is extant or may be designed to optimize this unusual surface. The computational studies on the complexation of ‘1’ and ‘6’ with RAD52 indicate the presence of a ubiquitous layer of interstitial water interactions with RAD52, yet these ligands are almost completely shielded from bulk solvent. The presence of these extensive interstitial water contacts further complicates hypotheses concerning which, if any, RAD52 functional groups are dominating the binding energy contacts. Rather, it may be that our HTS-generated hits possess the right combination of Van der Waals shape complementarity, and the ability to be both hydrogen bond donors and acceptors (with both interstitial waters and functional moieties) in the narrow DNA binding groove. Indeed, it may be that this shape complementarity and the ability to utilize the resident waters dominates the binding determinants (both for the identified ligands, as well as the native ssDNA substrate). Our iterative approach of compound discovery followed by the in silico screening was clearly successful in expanding the chemical space of our lead compounds, but more importantly provides a platform for strengthening the structure-activity relationship in an exceedingly challenging target pocket. Indeed, the discovery of the secondary plant metabolite, NP-004255, a macrocycle, as a means to effectively compete with a native substrate macromolecule (ssDNA and the ssDNA-RPA complex) may prove to be a new strategy in the field of disrupting protein-nucleic acid interactions.

Recently, Chandramouly and colleagues (Chandramouly et al., 2015) identified a small-molecule RAD52 inhibitor, 6-hydroxy-DL-dopa, that acts differently from the molecules reported here. This inhibitor interferes with the RAD52 oligomerization and the supramolecular assembly by an unresolved mechanism. It may act by binding at the RAD52 monomer-monomer interface, or at a different site on the protein and act allosterically. The existence of the distinct classes of RAD52 inhibitors, exemplified by ‘1’ and 6-hydroxy-DL-dopa, suggests that disrupting the RAD52-ssDNA interaction or the integrity of the RAD52 oligomeric ring bears negative consequences for the RAD52 cellular functions. Considering that the efficient homology search and the DNA strand annealing requires the two complementary DNA strands (or the complementary ssDNA-RPA complexes) to be wrapped around the two different RAD52 oligomeric rings (Grimme et al., 2010, Rothenberg et al., 2008), this is not surprising, and offers an exciting opportunities for development of more specific and potent agents for targeting recombination-deficient tumors.

While this manuscript was under review, two studies reporting small-molecule inhibitors of RAD52 were published. In the first study, Huang et al (Huang et al., 2016) carried out an HTS campaign to identify 17 compounds that inhibit RAD52-mediated annealing in vitro with IC₅₀ values ranging between 1.7 and 17 μM, physically bind RAD52, and selectively, albeit at high concentrations, inhibit the single-strand annealing pathway of DSB repair over homologous recombination. In another study, Sullivan et al (Sullivan et al., 2016) reported an in silico docking screen of a library of drug-like compounds. Among 36 predicted small-molecules, 9 compounds inhibited RAD52-ssDNA interaction in vitro, and 1 in cells. As with all previous publications, the authors screened a different libraries, resulting in compounds that represent a different chemical space from those that emerged from our campaign.

Since the expected role of RAD52 in the recovery of stalled replication forks in the absence of cellular checkpoint is to produce an intermediate that can be cleaved by MUS81/EME1 nuclease, we predicted that the ssDNA binding/annealing activity of RAD52 is required to fulfill this role. As expected, we found that ‘1’ and ‘6’ recapitulate inhibition of DSB formation by siRNA mediated RAD52 depletion (FIG. 5). In the case of ‘1’, 1 μM of the inhibitor was sufficient to achieve the same level in DSB reduction as siRNA treatment. Notably, no further inhibition was observed when the cells were treated with both siRNA and the small-molecule inhibitor suggesting that the effect is specific to RAD52. A higher concentration of ‘6’ was required to achieve the level of reduction in the RAD52/MUS81 dependent DSBs comparable with siRNA depletion of RAD52. This may be due to metabolic instability of this compound or due to potential off-target binding. For this reason, we placed an increased focus on ‘1’. The ability of ‘1’ to inhibit DSB formation, which is required for the recovery of damaged replication forks in the checkpoint deficient cells confirms that the ability of RAD52 to bind ssDNA is required for MUS81-dependent cleavage at stalled replication forks. Moreover, it is consistent with the mechanism we previously proposed whereby, in these cells, RAD52 used its ssDNA-binding activity to create a substrate for MUS81/EME1 and to recruit this structure selective nuclease. Consistent with our previous finding (Murfuni et al., 2013), RAD52 inhibition with ‘1’ acted additively with MUS 81 depletion eliciting an effect comparable with the RAD52 depletion. At 1 μM concentration of the inhibitor, approximately 40% of untreated and 60% of checkpoint-deficient, HU treated cells were dead (FIG. 6b). This is notable because the inhibitor interferes only with biochemical function of RAD52, namely its ability to bind ssDNA, while leaving the protein itself and its cellular concentration unperturbed, and also because even temporary loss of this biochemical activity during the exposure to replication stress is sufficient to exert the additive effect on viability. This result also illustrates the potentially powerful utility of these inhibitors in elucidating the function of RAD52 in the cell. We observed that inhibition of RAD52 during replication stress, which is induced by blocking DNA synthesis in the absence of the CHK1 activity, in a MUS81 knock-down background results in a comparable effect on viability as the concomitant depletion of both proteins. This observation strengthens the hypothesis that loss of MUS81 and RAD52 produces an additive lethal effect on replication stress (Murfuni et al., 2013) because, while RAD52 and MUS81 collaborate to resolve demised forks, MUS81 is subsequently required for resolution of recombination intermediates in a RAD52-independent pathway (FIG. 5 Supplement 1). More interestingly, ‘1’ was able to act at least additively with BRCA2 knock-down (FIG. 6b). An increase in the cell death when BRCA2 depletion was combined with ‘1’ was comparable to or even exceeding that of MUS81 depletion by siRNA. Notably, the effect of ‘1’ was further enhanced by replication stress induced by short HU treatment and concomitant CHK1 inhibition, as well as by a prolonged exposure to HU. Treatments inducing replication stress are widely used in cancer therapy (e.g. CPT, Gemcitabin). Therefore, RAD52 inhibitors could be useful in combination with drugs which elicit replication stress. Tumors in which MUS81 is mutated or downregulated have been described (Wu et al., 2011). While it is unclear whether the role RAD52 plays in supporting survival of the MUS81-deficient cells is akin to its role in supporting viability in the absence of BRCA1, BRCA2 or PALB2, RAD52 inhibitors may represent a new means of treatment for the MUS81-deficient tumors as well as the BRCA-deficient tumors.

In addition to its obvious uses in cancer therapy, the RAD52-ssDNA binding inhibitors can be utilized as molecular probes to assist in distinguishing the cellular pathways that depend on the main biochemical activity of RAD52. RAD52 may act together with other HR proteins, such as RAD51 paralogs to ensure formation of the active RAD51 nucleoprotein filament during RAD51-dependent HR. An understanding of the common players which might bind RAD52 in the absence of BRCA1 or BRCA2 and how they are regulated in the BRCA-deficient cells may require development of specific inhibitors of RAD52 protein-protein interactions and/or combining our inhibitors with other treatments that challenge homology directed DNA repair and replication.

Materials and Methods

Materials: The HPLC purified ssDNA substrate (Cy3-dT₃₀-Cy5), Target28Cy3 (T-28) (5′-ATAGTTATGGTGAGGACCC/iCy3/CTTTGTTTC-3′), Probe28Cy5 (P-28) (5′ GAAACAAAGGGGTCC/iCy5/TCACCATAACTAT-3′) Oligo28-REV (5′-(Cy5)-GCAATTAAGCTCAAGCCATCCGCAACG-(Cy3)-3′, Cy3-Oligo28-Cy5 (5′-CGTTGCGGATGGCTTAGAGCTTAATTGC-3′, and Poly dT100 were purchased from Integrated DNA Technologies (Coralville, Iowa). All chemicals were reagent grade (Sigma). All compounds were purchased from MicroSource and Sigma. Purity and structures of the purchased compounds were assessed from 1H NMR spectra collected on a Varian Unity Inova 600 MHz NMR spectrometer at 0.5 mM concentrations diluted into DMSO-d6.

Proteins: The 6× His-tagged human RAD52 protein was expressed and purified as previously described (Rothenberg et al., 2008), except a size exclusion chromatography (HiPrep 16/60 Sephacryl S-300 HR GE) step was added between the heparin and Resource S columns to remove low molecular weight impurities. RAD52 protein concentration was determined by measuring absorbance at 280 nm using extinction coefficient 40,380 M⁻¹ cm⁻¹. RPA protein was purified as described in (Henricksen et al., 1994, Grimme et al., 2010) (and its concentration was determined by measuring absorbance at 280 nm using extinction coefficient 84,000 M⁻¹ cm⁻¹.

High-Throughput Screening Assay for RAD52-ssDNA binding inhibition: HTS against the MicroSource Spectrum collection (Microsource, Gaylordsville, Conn.) was performed in nine 384 well plates. All measurements were carried out in the RAD52-HTS buffer containing 20 mM Hepes pH7.5, 1 mM DTT, and 0.1 mg/mL BSA. Each 384 well plate contained two columns of negative and positive controls as follow: Columns 1 and 24 were the positive controls, which contained 100 nM RAD52 (monomers) and 15 nM (molecules) Cy3-dT₃₀-Cy5 ssDNA in the RAD52-HTS buffer. Columns 2 and 23 in addition to 100 nM RAD52 and 15 nM Cy3-dT₃₀-Cy5 also contained 10 nM poly(dT)-100. These were designated as negative controls as 10 nM poly(dT)-100 was sufficient to fully inhibit formation of the wrapped RAD52-Cy3-dT₃₀-Cy5 complex under the selected experimental conditions (data not shown). Using a Multiflo dispenser (Biotek), 50 μL of the positive and negative controls were dispensed into their respective wells. Then 15 nM Cy3-dT₃₀-Cy5 and 100 nM RAD52 in RAD52-HTS buffer were dispensed into each well. Next, 1 μL of each compound at 833 μM in DMSO (for a final concentration of 15 μM compound) was dispensed into the wells in columns 3-22 using a Microlab Star liquid handling robot (Hamilton) and were mixed 3 times. Thus, 320 compounds were assayed per 384 well. The plate was incubated at 25° C. for 30 minutes and the fluorescent signal of the Cy3 (λ_{ex(Cy3)}=530 nm; λ_{em(Cy3)}=565 nm) and the Cy5 (λ_{em(Cy5)}=660 nm) dyes were recorded using a Wallac, Envision Manager. The apparent FRET was calculated as

$${FRET}_{app} = \frac{{I}_{Cy 5}}{{I}_{Cy 5} + {I}_{Cy 3}}$$

Assay performance was assessed across the screen using the following parameters: The signal-to-noise ratio

$$S / N = \frac{( {μ}_{n} - {μ}_{p} )}{{SD}_{n}} ,$$

the signal-to-background ratio

$$S / B = \frac{{μ}_{n}}{{μ}_{p}} ,$$

and the Z′-factor

$${Z}^{′} = 1 - \frac{3 * ( {SD}_{n} + {SD}_{p} )}{( {μ}_{n} - {μ}_{p} )} ,$$

where SDₚ and SDₙ are standard deviations, and μₙ and μₚ are means of the negative and positive control (Zhang et al., 1999).

Compounds from the wells that showed apparent FRET values at least 5 SD lower than the positive control were considered potential hits and were selected for the follow up analysis. Ninety six compounds were re-screened to assess reproducibility of hits. Twenty two of these compounds were removed due to high background signal. Twelve compounds, which showed reproducible reduction in FRET from screening of the individual plates and re-screening in cherry picked plates, were selected for biochemical validation.

WaterLOGSY NMR analysis of the compound binding to RAD52 and RPA proteins: Compound binding to RAD52 and RPA proteins was analyzed using water-ligand observation with gradient spectroscopy (WaterLOGSY) NMR experiments (Dalvit et al., 2001, Dalvit et al., 2000). The WaterLOGSY spectra of compounds in the presence of RAD52 or RPA were acquired using a water NOE mixing time of 1 s and a T₂ relaxation filter of 50 ms just before data acquisition to suppress the broad signals derived from protein. The protein buffer used in the experiments contains 10 mM Tris-d11, 75 mM KCl, 0.25 mM EDTA, pH 7.5, and 10% D₂0. All NMR data were acquired on a Bruker Avance II 800 MHz NMR spectrometer equipped with a sensitive cryoprobe and recorded at 25° C. The ¹H chemical shifts were referenced to 2,2-dimethyl-2-silapentane-5-sulfonate (DSS). NMR spectra were processed using NMR Pipe (Delaglio et al., 1995) and analysed using NMR View (Johnson and Blevins, 1994).

FRET-based DNA binding and annealing assays: FRET-based ssDNA binding, dsDNA binding, and annealing assays were carried out as previously described (Grimme et al., 2010, Grimme and Spies, 2011) using Cary Eclipse spectrofluorimeter (Varian) at 25° C. in buffer containing 30 mM Tris-Acetate pH7.5, 1 mM DTT, and 0.1 mg/mL BSA. Cy3 dye was excited at 530 nm and its emission was monitored at 565 nm. Emission of Cy5 acceptor fluorophore excited through the energy transfer from Cy3 donor is monitored at 660 nm simultaneously with emission of Cy3 dye. Both the excitation and the emission slit widths were set to 10 nm.

To confirm that selected compounds inhibit RAD52 mediated binding and wrapping of ssDNA, compounds were titrated into stoichiometric complex containing 1 nM T30 and 8 nM RAD52. All experiments were performed in triplicates, and the data are shown as averages and standard deviations for three independent measurements. To remove possible experimental artifacts associated with chromogenic or fluorogenic compounds, as well as with the compounds that may quench or enhance Cy3 or Cy5 fluorescence, we also performed control titrations whereby we titrated each compound into 1 nM Cy3-dT₃₀-Cy5 in the absence of RAD52. For each compound concentration we subtracted the difference in the FRET signal in the presence and absence of the compound from the respective FRET signal in the presence of RAD52. The FRET signal corrected for the compound fluorescence was calculated using the equation:

$${FRET}_{app} = \frac{4.2 * {I}_{Cy 5}}{4.2 * {I}_{Cy 5} + 1.7 * {I}_{Cy 3}}$$

as previously described (Grimme et al., 2010, Grimme and Spies, 2011) and plotted as a function of compound concentration and fitted to the following inhibition model”

$$FRET ( [ small molecule ] ) = \frac{{FRET}_{0} - {FRET}_{min}}{1 + {10}^{( ( LogIC 50 - LOG ( [ small molecule ] ) + HillSlope )}} ,$$

where FRET₀ is the initial FRET value of RAD52-ssDNA complex in the absence of the compound and FRETₘᵢₙ is the FRET value at complete inhibition. FRET values were calculated as an average of three or more independent annealing reactions plotted against the concentration of the compound. Inhibition of the RAD52-dsDNA interaction was assayed in a similar experiment, except the stoichiometric complexes containing 1 nM molecules of Cy3-Oligo28-Cy5 duplex DNA and 10 nM RAD52. To assess if selected compounds inhibit RPA-ssDNA mediated binding and wrapping by RAD52 we titrated compounds into stoichiometric complexes containing 1 nM T30, 1 nM RPA, and 10 nM RAD52. In all experiments, the FRET values for each data point were corrected for the effects of the compounds on the respective substrate in the absence of RAD52.

Annealing of complementary oligonucleotides by RAD52 was monitored under identical conditions as the binding assays described above. For each assay, the reaction master mixture containing 8 nM RAD52 protein in the presence and absence of the compounds at varying concentrations was prepared at room temperature and divided into two half reactions. Following baseline buffer and protein measurements, 0.5 nM of T-28 ssDNA substrate was added to the reaction cuvette and the signal was allowed to stabilize. The annealing reaction was initiated upon addition of the second half-reaction pre-incubated with 0.5 nM P-28 ssDNA substrate. The fluorescence of Cy3 and Cy5 were measured simultaneously over the reaction time course (500 s). FRETₐₚₚ values were calculated as an average of three or more independent annealing reactions plotted against time (s). The average FRET values were fitted to a double exponential to calculate the final extent of annealing using Graphpad Prism4 software. The calculated annealing extent was plotted as a function of compound concentration and fitted to the same model as we used to determine IC₅₀ values for the inhibition of DNA annealing.

Docking, Molecular Mechanics (MM) and Generalized Born (GB)/Surface Area (SA) (MMGB/SA)-based Free Energy Scoring for RAD52 Ligands: Our initial computational workflow employed a combination of classical docking, using the Triangle Matcher approach and scoring using the London dG scoring function (an empirical scoring function which attempts approximate the binding energy of the docked ligand) in MOE 2013.08 (Molecular Operating Environment (MOE) 2013.08, 2013), followed by force field (MMFF94x (Halgren, 1996))-based ligand refinement and finally rescoring using an MM/GBSA-based approach (which is described in more detail below). Initially, top scoring poses in either Triangle Matcher (Placement), London dG (affinity scoring function) or MM/GBSA (physics-based scoring) were retained for further analysis. Often the top scoring poses from each metric were highly distinct from one another, suggesting that a generally poor consensus between the different metrics used in this early phase of the work flow. This lack of consensus in the scoring of the possible ligand binding in the sub-pockets within the DNA-binding groove of RAD52 (PDB: 1KNO) motivated us to apply the much more computationally rigorous all atom simulated annealing studies, which are detailed below. All lead ligands were subjected to docking using MOE 2013.08 to a portion of the DNA binding groove of RAD52 spanning nearly a quarter of the circumference (3 adjacent monomers of the protein ring). The top 30 poses for each docked and scored (London dG scoring function) were subjected to energy minimization with a rigid RAD52 receptor using the MMFF94x force field, followed by rescoring (in order to estimate the AG of binding) of each distinct pose with the MM/GBSA methodology (Naïm, et al., 2007), which includes an implicit solvation energy calculation and captures changes in the solvent exposed surface area of the pose, which is a highly parameterized version of the popular MM/PBSA and MM/GBSA methodologies (Steinbrecher and Labahn, 2010, Wang and Kollman, 2000).

All Atom Simulated Annealing Energy Minimization with the YASARA2 Knowledge-based Forcefield, and Rescoring with VINA: The all atom Simulated Annealing Energy Minimization (here referred to SAEM for brevity), which is followed by a local docking protocol (as described below) is a customized protocol that was automated with a script using the Python-based Yanaconda scripting language, and use of the Yamber03 knowledge-based force field (Krieger et al., 2004). Briefly, each complex was placed into a simulation cell and solvated, and charge-neutralized to yield physiological conditions, followed by an optimization of the solvent and H-bonding network, and finally a phased simulated annealing minimization was performed (a similar process is described in Whalen et al., 2011 (Whalen et al., 2011)). No restraints were placed in any of these systems (i.e. all atoms in the ligand and the entire RAD52 complex, ions and solvent were free to move in the simulation). The affinity of the ligand in this optimized complex was then determined by scoring with AutoDock VINA (Trott and Olson, 2010). Water molecules that were interstitial were automatically retained in the VINA scoring.

Neutral Comet Assay: To induce RAD52-MUS81-dependent cleavage at arrested replication forks (Murfuni et al., 2013), hTERT-immortalized wild-type human fibroblasts (GM01604) were treated with 2 mM HU and 300 nM UCN01 for 6 h, in the presence and absence of varying concentrations of ‘1’ or ‘6’. Where indicated, the GM01604 cells were cells were transfected with siRNAs directed against GFP (Ctrl), or against RAD52 (Qiagen) 48 hours prior to induction of the replication stress and/or inhibitor treatment. After that cells were subjected to neutral comet assay as described in Murfuni et al (Murfuni et al., 2013). Slides were analyzed by a computerized image analysis system (Comet IV, Perceptive UK). To assess the quantity of DNA damage, computer-generated tail moment values (tail length×fraction of total DNA in the tail) were used. Apoptotic cells (smaller comet head and extremely larger comet tail) were excluded from the analysis to avoid artificial enhancement of tail moment. A minimum of 100 cells were analyzed for each compound concentration point.

Cell Viability Live/Dead Assays: GM01604 cells were transfected with siRNAs directed against GFP (Ctrl), or against MUS81 (Qiagen), BRCA2 (Sigma-Aldrich), and RAD52 (Qiagen) 48 hours prior to addition of 1 μM ‘1’. Where indicated, the conditions of pathological replication were induced by treating cells with 2 mM HU and 300 nM UCN01 for 6 h or by a 18 hours treatment with 2 mM HU, in the presence or absence of ‘1’. Viability was evaluated by the LIVE/DEAD assay (Sigma-Aldrich) according to the manufacturer's instructions. Cell number was counted in randomly chosen fields and expressed as percent of dead cells (number of red nuclear stained cells divided by the total cell number) corrected for the cell loss observed in the population. For each time point, at least 200 cells were counted.

In Silico Screening Leading to Identification of Novel Inhibitor NP-004255 Using SAR from HTS: The AnalytiCon Discovery MEGx Natural Products Screen Library, which is the in silico version of an actual library of purified natural products from plant, fungal and microbial sources, which is available for purchase, was subjected to an in silico screening campaign. The campaign was designed based on the ability to optimally minimize false positives and false negatives, and to maximize true positives and true negatives. More specifically, the experimental hits identified in the HTS campaign described above, constitute the true positives, while specifically selected decoy compounds constitute the true negatives. Decoy compounds were generated using the Database of Useful Decoys-Enhanced (DUD-E) website (Mysinger et al., 2012). Decoys are compounds that resemble active ligands in physicochemical properties, but are distinct in chemical topology to true binders, so that separation bias is avoided (Huang et al., 2006). Decoys are property-matched to compounds of interest using molecular weight, estimated water-octanol partition coefficient (miLogP), rotatable bonds, hydrogen bond acceptors, hydrogen bond donors, and net charge (Mysinger et al., 2012). An average of 50 decoys are obtained for each ligand.

In order to validate the selected protocol (FIG. 1b ), we employed a statistical method in which ‘Receiver Operating Characteristic’ or ROC curves are used to optimize the balance of true positives, false positives, true negatives and false negatives (Varnek et al., 2008). ROC curves were created using MatLab (R2015a; Mathworks, Natick, Mass., USA) from scoring ranks of active versus inactive poses for each of the best HTS hits (Varnek et al., 2008). This plot represents the percentage of true positives versus percentage of false positives for a wide range of choices of score cutoffs. This procedure also allows the determination of the best score threshold for cutoff of compounds regarding the particular protein target.

Specific Docking and Scoring Procedures: A database containing the AnalytiCon Natural Products compound library, and a control selected from the initial HTS hits, were created and preprocessed for virtual docking (as described above). The top 30 final poses, generated using the Dock utility of MOE (as described above) were written to an output database. Poses of all compounds were ranked based on their scores. Compounds with the poses most favorable to binding, i.e. the poses with the lowest energy scores from the London dG scoring function were selected for further analysis. Those poses with better scores than the highest scoring pose of our control (ie, a “true positive” in the ROC curve context) were selected, and then subjected to a refining docking step involving force field-based energy minimization with the MMFF94x force field in MOE. Binding energies were ranked, and evaluated.

Construction of Receiver Operator Characteristic (ROC) Curves: The docking scores (kcal/mol) were used for determining the ROC threshold values (see (DeLong et al., 1988), for precise description of the how to determine the threshold value). Each original compound of interest and its poses were to be the only “predicted positives”, and the DUDs (decoys) and its poses were to be the “predicted negatives”; any poses above the threshold were to be the “actual positives” and the poses below the threshold were to be the “actual negatives”. The curves were analyzed using the metric of area under the curves (AUC) (DeLong et al., 1988). The scores of the poses for the most active compounds exhibited bimodal frequency distribution (FIG. 8b), and the docking protocols' ability to distinguish between active compounds and decoys was verified (FIG. 8c). We have determined a protocol for sorting compounds of interest among a database that provides reliable results with high cutoff limits,

### REFERENCES

ABBAS, T., KEATON, M. A. & DUTTA, A. 2013. Genomic Instability in Cancer. Cold Spring Harbor Perspectives in Biology, 5.

BENSON, F. E., BAUMANN, P. & WEST, S. C. 1998. Synergistic actions of Rad51 and Rad52 in recombination and DNA repair. Nature, 391, 401-4.

BUISSON, R., NIRAJ, J., PAUTY, J., MAITY, R., ZHAO, W., COULOMBE, Y., SUNG, P. & MASSON, J. Y. 2014. Breast cancer proteins PALB2 and BRCA2 stimulate polymerase eta in recombination-associated DNA synthesis at blocked replication forks. Cell Rep, 6, 553-64.

CARREIRA, A., HILARIO, J., AMITANI, I., BASKIN, R. J., SHIVA M. K. K., VENKITARAMAN, A. R. & KOWALCZYKOWSKI, S. C. 2009. The BRC Repeats of BRCA2 Modulate the DNA-Binding Selectivity of RAD51. Cell, 136, 1032-1043.

CHANDRAMOULY, G., MCDEVITT, S., SULLIVAN, K., KENT, T., LUZ, A., GLICKMAN, J. F., ANDRAKE, M., SKORSKI, T. & POMERANTZ, R. T. 2015. Small-Molecule Disruption of RAD52 Rings as a Mechanism for Precision Medicine in BRCA-Deficient Cancers. Chem Biol, 22, 1491-504.

CHEN, R. & WOLD, M. S. 2014. Replication protein A: Single-stranded DNA's first responder: Dynamic DNA-interactions allow replication protein A to direct single-strand DNA intermediates into different pathways for synthesis or repair. Bioessays, 36, 1156-61.

CHUN, J., BUECHELMAIER, E. S. & POWELL, S. N. 2013. Rad51 Paralog Complexes BCDX2 and CX3 Act at Different Stages in the BRCA1-BRCA2-Dependent Homologous Recombination Pathway. Molecular and Cellular Biology, 33, 387-395.

COUEDEL, C., MILLS, K. D., BARCHI, M., SHEN, L., OLSHEN, A., JOHNSON, R. D., NUSSENZWEIG, A., ESSERS, J., KANAAR, R., LI, G. C., ALT, F. W. & JASIN, M. 2004. Collaboration of homologous recombination and nonhomologous end-joining factors for the survival and integrity of mice and cells. Genes Dev, 18, 1293-304.

CRAMER-MORALES, K., NIEBOROWSKA-SKORSKA, M., SCHEIBNER, K., PADGET, M., IRVINE, D. A., SLIWINSKI, T., HAAS, K., LEE, J., GENG, H., ROY, D., SLUPIANEK, A., RASSOOL, F. V., WASIK, M. A., CHILDERS, W., COPLAND, M., MUSCHEN, M., CIVIN, C. I. & SKORSKI, T. 2013. Personalized synthetic lethality induced by targeting RAD52 in leukemias identified by gene mutation and expression profile. Blood, 122, 1293-304.

DALVIT, C., FOGLIATTO, G., STEWART, A., VERONESI, M. & STOCKMAN, B. 2001. WaterLOGSY as a method for primary NMR screening: practical aspects and range of applicability. J Biomol NMR, 21, 349-59.

DALVIT, C., PEVARELLO, P., TATO, M., VERONESI, M., VULPETTI, A. & SUNDSTROM, M. 2000. Identification of compounds with binding affinity to proteins via magnetization transfer from bulk water. J. Biomol. NMR, 18, 65-68.

DELAGLIO, F., GRZESIEK, S., VUISTER, G., ZHU, G., PFEIFER, J. & BAX, A. 1995. NMRPipe: A multidimensional spectral processing system based on UNIX pipes. Journal of Biomolecular NMR, 6, 277-293.

DELONG, E. R., DELONG, D. M. & CLARKE-PEARSON, D. L. 1988. Comparing the areas under two or more correlated receiver operating characteristic curves: a nonparametric approach. Biometrics, 44, 837-45.

DOE, C. L., OSMAN, F., DIXON, J. & WHITBY, M. C. 2004. DNA repair by a Rad22-Mus81-dependent pathway that is independent of Rhp51. Nucleic Acids Res, 32, 5570-81.

DRAY, E., ETCHIN, J., WIESE, C., SARO, D., WILLIAMS, G. J., HAMMEL, M., YU, X., GALKIN, V. E., LIU, D., TSAI, M. S., SY, S. M., SCHILD, D., EGELMAN, E., CHEN, J. & SUNG, P. 2010. Enhancement of RAD51 recombinase activity by the tumor suppressor PALB2. Nat Struct Mol Biol, 17, 1255-9.

ELLINGSON, S. R., MIAO, Y., BAUDRY, J. & SMITH, J. C. 2015. Multi-conformer ensemble docking to difficult protein targets. J Phys Chem B, 119, 1026-34.

FENG, Z., SCOTT, S. P., BUSSEN, W., SHARMA, G. G., GUO, G., PANDITA, T. K. & POWELL, S. N. 2011. Rad52 inactivation is synthetically lethal with BRCA2 deficiency. Proc Natl Acad Sci USA, 108, 686-91.

GRIMME, J. M., HONDA, M., WRIGHT, R., OKUNO, Y., ROTHENBERG, E., MAZIN, A. V., HA, T. & SPIES, M. 2010. Human Rad52 binds and wraps single-stranded DNA and mediates annealing via two hRad52-ssDNA complexes. Nucleic Acids Res, 38, 2917-30.

GRIMME, J. M. & SPIES, M. 2011. FRET-based assays to monitor DNA binding and annealing by Rad52 recombination mediator protein. Methods Mol Biol, 745, 463-83.

HALGREN, T. A. 1996. Merck molecular force field. I. Basis, form, scope, parameterization, and performance of MMFF94. Journal of Computational Chemistry, 17, 490-519.

HEAD, M. S. 2010. Docking: a domesday report. In: Kenneth M. Merz, Jr et al. (eds.) Drug Design. pp. 98-119., Cambridge University Press.

HENRICKSEN, L. A., UMBRICHT, C. B. & WOLD, M. S. 1994. Recombinant replication protein A: expression, complex formation, and functional characterization. J Biol Chem, 269, 11121-32.

HEYER, W.-D. 2015a. Regulation of Recombination and Genomic Maintenance. Cold Spring Harbor Perspectives in Biology, 7.

HEYER, W. D. 2015b. Regulation of recombination and genomic maintenance. Cold Spring Harb Perspect Biol, 7, a016501.

HONDA, M., OKUNO, Y., YOO, J., HA, T. & SPIES, M. 2011. Tyrosine phosphorylation enhances RAD52-mediated annealing by modulating its DNA binding. EMBO J, 30, 3368-82.

HUANG, F., GOYAL, N., SULLIVAN, K., HANAMSHET, K., PATEL, M., MAZINA, O. M., WANG, C. X., AN, W. F., SPOONAMORE, J., METKAR, S., EMMITTE, K. A., COCKLIN, S., SKORSKI, T. & MAZIN, A. V. 2016. Targeting BRCA1- and BRCA2-deficient cells with RAD52 small molecule inhibitors. Nucleic Acids Res, 44, 4189-99.

HUANG, N., SHOICHET, B. K. & IRWIN, J. J. 2006. Benchmarking sets for molecular docking. J Med Chem, 49, 6789-801.

JASIN, M. & ROTHSTEIN, R. 2013. Repair of strand breaks by homologous recombination. Cold Spring Harb Perspect Biol, 5, a012740.

JENSEN, R. B., CARREIRA, A. & KOWALCZYKOWSKI, S. C. 2010. Purified human BRCA2 stimulates RAD51-mediated recombination. Nature, 467, 678-83.

JOHNSON, B. & BLEVINS, R. 1994. NMR View: A computer program for the visualization and analysis of NMR data. Journal of Biomolecular NMR, 4, 603-614.

KAGAWA, W., KAGAWA, A., SAITO, K., IKAWA, S., SHIBATA, T., KURUMIZAKA, H. & YOKOYAMA, S. 2008. Identification of a second DNA binding site in the human Rad52 protein. J Biol Chem, 283, 24264-73.

KAGAWA, W., KURUMIZAKA, H., IKAWA, S., YOKOYAMA, S. & SHIBATA, T. 2001. Homologous pairing promoted by the human Rad52 protein. J Biol Chem, 276, 35201-8.

KAGAWA, W., KURUMIZAKA, H., ISHITANI, R., FUKAI, S., NUREKI, O., SHIBATA, T. & YOKOYAMA, S. 2002. Crystal structure of the homologous-pairing domain from the human Rad52 recombinase in the undecameric form. Mol Cell, 10, 359-71.

KASS, E. M. & JASIN, M. 2010. Collaboration and competition between DNA double-strand break repair pathways. FEBS Lett, 584, 3703-8.

KIM, H. & D'ANDREA, A. D. 2012. Regulation of DNA cross-link repair by the Fanconi anemia/BRCA pathway. Genes Dev, 26, 1393-408.

KOWALCZYKOWSKI, S. C. 2015. An Overview of the Molecular Mechanisms of Recombinational DNA Repair. Cold Spring Harb Perspect Biol, 7.

KRIEGER, E., DARDEN, T., NABUURS, S. B., FINKELSTEIN, A. & VRIEND, G. 2004. Making optimal use of empirical energy functions: force-field parameterization in crystal space. Proteins, 57, 678-83.

LI, X. & HEYER, W. D. 2008. Homologous recombination in DNA repair and DNA damage tolerance. Cell Research, 18, 99-113.

LIU, J., DOTY, T., GIBSON, B. & HEYER, W. D. 2010. Human BRCA2 protein promotes RAD51 filament formation on RPA-covered single-stranded DNA. Nat Struct Mol Biol, 17, 1260-2.

LLOYD, J. A., FORGET, A. L. & KNIGHT, K. L. 2002. Correlation of biochemical properties with the oligomeric state of human rad52 protein. J Biol Chem, 277, 46172-8.

LLOYD, J. A., MCGREW, D. A. & KNIGHT, K. L. 2005. Identification of residues important for DNA binding in the full-length human Rad52 protein. J Mol Biol, 345, 239-49.

LOK, B. H., CARLEY, A. C., TCHANG, B. & POWELL, S. N. 2012. RAD52 inactivation is synthetically lethal with deficiencies in BRCA1 and PALB2 in addition to BRCA2 through RAD51-mediated homologous recombination. Oncogene.

LOK, B. H. & POWELL, S. N. 2012. Molecular pathways: understanding the role of Rad52 in homologous recombination for therapeutic advancement. Clin Cancer Res, 18, 6400-6.

MCILWRAITH, M. J. & WEST, S. C. 2008. DNA repair synthesis facilitates RAD52-mediated second-end capture during DSB repair. Mol Cell, 29, 510-6.

MOLECULAR OPERATING ENVIRONMENT (MOE) 2013.08, C. C. G. I., 1010 SHERBOOKE ST. WEST, SUITE #910, MONTREAL, QC, CANADA, H3A 2R7 2013.

MORTENSEN, U. H., ERDENIZ, N., FENG, Q. & ROTHSTEIN, R. 2002. A molecular genetic dissection of the evolutionarily conserved N terminus of yeast Rad52. Genetics, 161, 549-62.

MORTENSEN, U. H., LISBY, M. & ROTHSTEIN, R. 2009. Rad52. Current Biology, 19, R676-R677.

MOYNAHAN, M. E. & JASIN, M. 2010. Mitotic homologous recombination maintains genomic stability and suppresses tumorigenesis. Nat Rev Mol Cell Biol, 11, 196-207.

MURFUNI, I., BASILE, G., SUBRAMANYAM, S., MALACARIA, E., BIGNAMI, M., SPIES, M., FRANCHITTO, A. & PICHIERRI, P. 2013. Survival of the replication checkpoint deficient cells requires MUS81-RAD52 function. PLoS Genet, 9, e1003910.

MYSINGER, M. M., CARCHIA, M., IRWIN, J. J. & SHOICHET, B. K. 2012. Directory of useful decoys, enhanced (DUD-E): better ligands and decoys for better benchmarking. J Med Chem, 55, 6582-94.

NAIM, M., BHAT, S., RANKIN, K. N., DENNIS, S., CHOWDHURY, S. F., SIDDIQI, I., DRABIK, P., SULEA, T., BAYLY, C. I., JAKALIAN, A. & PURISIMA, E. O. 2007. Solvated Interaction Energy (SIE) for Scoring Protein-Ligand Binding Affinities. 1. Exploring the Parameter Space. Journal of Chemical Information and Modeling, 47, 122-133.

OAKLEY, G. G. & PATRICK, S. M. 2010. Replication protein A: directing traffic at the intersection of replication and repair. Front Biosci, 15, 883-900.

PARK, J. Y., ZHANG, F. & ANDREASSEN, P. R. 2014. PALB2: the hub of a network of tumor suppressors involved in DNA damage responses. Biochim Biophys Acta, 1846, 263-75.

PRAKASH, R., ZHANG, Y., FENG, W. & JASIN, M. 2015. Homologous Recombination and Human Health: The Roles of BRCA1, BRCA2, and Associated Proteins. Cold Spring Harbor Perspectives in Biology, 7.

RIJKERS, T., VAN DEN OUWELAND, J., MOROLLI, B., ROLINK, A. G., BAARENDS, W. M., VAN SLOUN, P. P., LOHMAN, P. H. & PASTINK, A. 1998. Targeted inactivation of mouse RAD52 reduces homologous recombination but not resistance to ionizing radiation. Mol Cell Biol, 18, 6423-9.

ROTHENBERG, E., GRIMME, J. M., SPIES, M. & HA, T. 2008. Human Rad52-mediated homology search and annealing occurs by continuous interactions between overlapping nucleoprotein complexes. Proc Natl Acad Sci USA, 105, 20274-9.

SCHLACHER, K., CHRIST, N., SIAUD, N., EGASHIRA, A., WU, H. & JASIN, M. 2011. Double-strand break repair-independent role for BRCA2 in blocking stalled replication fork degradation by MRE11. Cell, 145, 529-42.

SCHLACHER, K., WU, H. & JASIN, M. 2012. A distinct replication fork protection pathway connects Fanconi anemia tumor suppressors to RAD51-BRCA1/2. Cancer Cell, 22, 106-16.

SINGLETON, M. R., WENTZELL, L. M., LIU, Y., WEST, S. C. & WIGLEY, D. B. 2002a. Structure of the single-strand annealing domain of human RAD52 protein. Proceedings of the National Academy of Sciences, 99, 13492-13497.

SINGLETON, M. R., WENTZELL, L. M., LIU, Y., WEST, S. C. & WIGLEY, D. B. 2002b. Structure of the single-strand annealing domain of human RAD52 protein. Proc Natl Acad Sci USA, 99, 13492-7.

SOGA, S., SHIRAI, H., KOBORI, M. & HIRAYAMA, N. 2007. Use of amino acid composition to predict ligand-binding sites. J Chem Inf Model, 47, 400-6.

STASIAK, A. Z., LARQUET, E., STASIAK, A., MULLER, S., ENGEL, A., VAN DYCK, E., WEST, S. C. & EGELMAN, E. H. 2000. The human Rad52 protein exists as a heptameric ring. Curr Biol, 10, 337-40.

STEINBRECHER, T. & LABAHN, A. 2010. Towards accurate free energy calculations in ligand protein-binding studies. Curr Med Chem, 17, 767-85.

SULLIVAN, K., CRAMER-MORALES, K., MCELROY, D. L., OSTROV, D. A., HAAS, K., CHILDERS, W., HROMAS, R. & SKORSKI, T. 2016. Identification of a Small Molecule Inhibitor of RAD52 by Structure-Based Selection. PLoS One, 11, e0147230.

THORSLUND, T., MCILWRAITH, M. J., COMPTON, S. A., LEKOMTSEV, S., PETRONCZKI, M., GRIFFITH, J. D. & WEST, S. C. 2010. The breast cancer tumor suppressor BRCA2 promotes the specific targeting of RAD51 to single-stranded DNA. Nat Struct Mol Biol, 17, 1263-5.

TROTT, O. & OLSON, A. J. 2010. AutoDock Vina: improving the speed and accuracy of docking with a new scoring function, efficient optimization, and multithreading. J Comput Chem, 31, 455-61.

VAN DYCK, E., STASIAK, A. Z., STASIAK, A. & WEST, S. C. 2001. Visualization of recombination intermediates produced by RAD52-mediated single-strand annealing. EMBO Rep, 2, 905-9.

VARNEK, A., TROPSHA, A. & ROYAL SOCIETY OF CHEMISTRY (GREAT BRITAIN). 2008. Chemoinformatics approaches to virtual screening, Cambridge, RSC Pub.

VEJRUP-HANSEN, R., MIZUNO, K., MIYABE, I., FLECK, O., HOLMBERG, C., MURRAY, J. M., CARR, A. M. & NIELSEN, O. 2011. Schizosaccharomyces pombe Mms1 channels repair of perturbed replication into Rhp51 independent homologous recombination. DNA Repair (Amst), 10, 283-95.

WANG, W. & KOLLMAN, P. A. 2000. Free energy calculations on dimer stability of the HIV protease using molecular dynamics and a continuum solvent model. J Mol Biol, 303, 567-82.

WARREN, G. L., ANDREWS, C. W., CAPELLI, A. M., CLARKE, B., LALONDE, J., LAMBERT, M. H., LINDVALL, M., NEVINS, N., SEMUS, S. F., SENGER, S., TEDESCO, G., WALL, I. D., WOOLVEN, J. M., PEISHOFF, C. E. & HEAD, M. S. 2006. A critical assessment of docking programs and scoring functions. J Med Chem, 49, 5912-31.

WHALEN, K. L., CHANG, K. M. & SPIES, M. A. 2011. Hybrid Steered Molecular Dynamics-Docking: An Efficient Solution to the Problem of Ranking Inhibitor Affinities Against a Flexible Drug Target. Mol Inform, 30, 459-471.

WOLD, M. S. 1997. Replication Protein A: A heterotrimeric, single-stranded DNA-binding protein required for eukaryotic DNA metabolism. Annu Rev Biochem 66, 61-92.

WU, F., SHIRAHATA, A., SAKURABA, K., KITAMURA, Y., GOTO, T., SAITO, M., ISHIBASHI, K., KIGAWA, G., NEMOTO, H., SANADA, Y. & HIBI, K. 2011. Downregulation of Mus81 as a novel prognostic biomarker for patients with colorectal carcinoma. Cancer Sci, 102, 472-7.

XIA, F., TAGHIAN, D. G., DEFRANK, J. S., ZENG, Z. C., WILLERS, H., ILIAKIS, G. & POWELL, S. N. 2001. Deficiency of human BRCA2 leads to impaired homologous recombination but maintains normal nonhomologous end joining. Proceedings of the National Academy of Sciences of the United States of America, 98, 8644-8649.

YAMAGUCHI-IWAI, Y., SONODA, E., BUERSTEDDE, J. M., BEZZUBOVA, O., MORRISON, C., TAKATA, M., SHINOHARA, A. & TAKEDA, S. 1998. Homologous recombination, but not DNA repair, is reduced in vertebrate cells deficient in RAD52. Mol Cell Biol, 18, 6430-5.

YANEZ, R. J. & PORTER, A. C. 2002. Differential effects of Rad52p overexpression on gene targeting and extrachromosomal homologous recombination in a human cell line. Nucleic Acids Res, 30, 740-8.

YANG, H. J., LI, Q. B., FAN, J., HOLLOMAN, W. K. & PAVLETICH, N. P. 2005. The BRCA2 homologue Brh2 nucleates RAD51 filament formation at a dsDNA-ssDNA junction. Nature, 433, 653-657.

YAP, J. L., CHAUHAN, J., JUNG, K.-Y., CHEN, L., PROCHOWNIK, E. V. & FLETCHER, S. 2012. Small-molecule inhibitors of dimeric transcription factors: Antagonism of protein-protein and protein-DNA interactions. MedChemComm, 3, 541-551.

ZHANG, J. H., CHUNG, T. D. Y. & OLDENBURG, K. R. 1999. A simple statistical parameter for use in evaluation and validation of high throughput screening assays. Journal of Biomolecular Screening, 4, 67-73.

### Example 2

### Identification of Additional Compounds

Additional compounds were identified and tested using methods similar to the methods of Example 1, as illustrated in Table 2.

TABLE 2

| | | | IC₅₀ (DNA binding); |
|---|---|---|---|
| | | | FRET value |
| No. | Name | Structure | at saturation |
| R20 | 2-hydroxy-6-methyl-2-(trifluoromethyl)- 2,3-dihydro-4H-chromen-4-one | 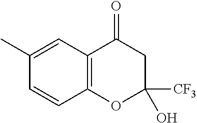 | 938 ± 12 nM; 0.49 ± 0.01 |
| R21 | 2-hydroxy-2-(trifluoromethyl)-2,3- dihydro-4H-chromen-4-one oxime | 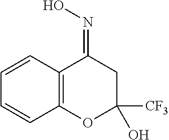 | 76.0 ± 5.57 μM; 0.483 ± 0.01 |
| R22 | 2-hydroxy-5,5-dimethyl-2- (trifluoromethyl)tetrahydro-4H-pyran-one | 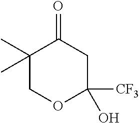 | 184 ± 12 μM; 0.49 ± 0.01 |
| R23 | 3-hydroxy-3-(2-hydroxypropyl)-1-methyl- 1,3-dihydro-2H-indol-2-one | 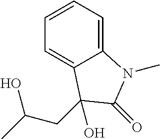 | 84.1 ± 1.04 μM; 0.39 ± 0.01 |
| R24 | 2-(1-hydroxy-1methylethyl)-2,3-dihydro- 7H-furo[3,2-g]crhomen-7-one | 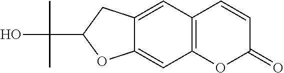 | No inhibition |
| R25 | 6-(1,3-benzodioxol-5-yl)-3-(benzylthio)- 6,7-dihydro[1,2,4]triazino[5,6-d][3,1] benzoxazepine | 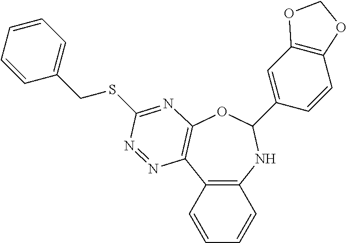 | 1.6 ± 0.15 μM; 0.45 ± 0.01 n = 3 |
| R26 | 3-(benzylthio)-6-(4-ethoxyphenyl)-6,7- dihydro[1,24]triazino[5,6-d][3,1] benzoxazepine | 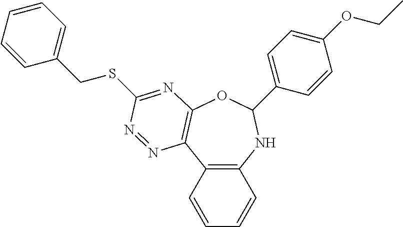 | 1.9 ± 0.5 μM; 0.46 ± 0.01 n = 3 |
| R27 | 2-[(9H-purin-6-ylthio)methyl]-3-[3- (trifluoromethyl)phenyl]-4(3H)- qhinazolinone | 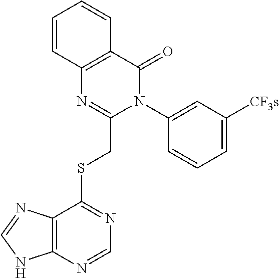 | Insoluble in 30 mM Tris-Acetate pH 7.5, 1 mM DTT at 115 μM |
| R28 | 7-{(3-ethoxy-4-methyoxyphenyl)[(4- methyl-2-pyridinyl)amino]methyl}-2- methyl-8-quinolinol | 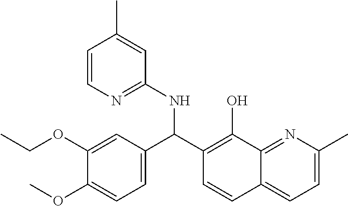 | Insoluble in 30 mM Tris-Acetate pH 7.5, 1 mM DTT at 10 μM |
| R29 | N-(2-ethoxyphenyl)-2-{[(4- methoxyphenyl)acetyl]amino)-5,6- dihydro-4H-cyclopenta[b]thiophene-3- carboxamide | 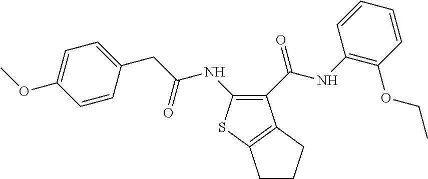 | Compound is insoluble in DMSO at 25 mM |
| R30 | Tetrahydro-2-furanylmethyl 4-(4- hydroxy-3-methoxyphenyl)-2,7,7- trimethyl-5oxo-1,4,5,6,7,8-hexahydro- 3-quinolinecarboxylate | 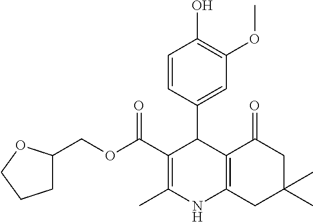 | 1.303 ± 0.19 μM; 0.59 ± 0.01 n = 3 |
| R31 | 1-(4-chlorophenyl)-N-ethyl-7,7- dimethyl-2,5-dioxo-N-phenyl- 1,2,5,6,7,8-hexahydro-3- quinolinecaroxamide | 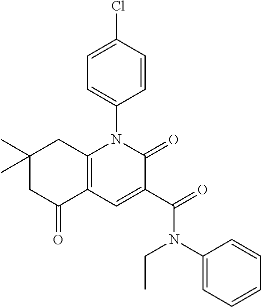 | |
| R32 | N-[1-(anilinocarbonyl)-2-methylpropyl]- 2-[(4-methoxybenzoyl)amino]benzamide | 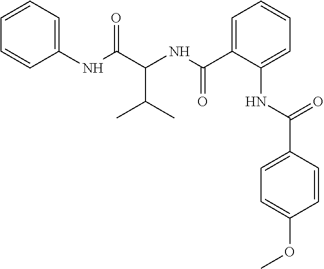 | 1.246 ± 0.34 μM; 0.45 ± 0.01 n = 3 |
| R33 | 4-hydroxy-N′-(4-hydroxy-3- methoxybenzyliden)pentanohydrazide | 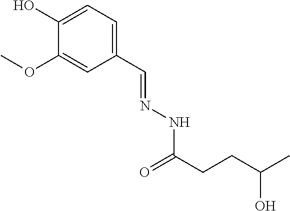 | 0.849 ± 0.21 μM; 0.56 ± 0.01 n = 3 |

Compounds R20-R24 were identified by screening the local ChemBridge database against one of 30 pharmacophore models. Compounds R20-R24 were selected based on having the lowest number of rotatables and the lowest RMSD value relative to the 30 filtering model. The results identified 19 compounds from which compounds R20-R24 were selected for testing.

Compounds R25-R35 were selected by using the hRA052(1-209) crystal structure and by using implicate force field docking into a druggable pocket of hRA052 that was identified by the present inventors. The length of the simulations was X. The compounds were found to exhibit two predominant orientations in which they overlap in what is thought to be the single-stranded binding pocket of hRA052. Because the docking is based on force field, the final calculated binding energies provide an approximate rank-ordering from the DNA binding IC₅₀ values. The compound having the structure (smiles=O1c2c(C[C@@H](OC(═O)c3cc(O)c(O)c(O)c3)[C@H]1c1cc(O)c(O)cc1)c(O)cc(O)c2) was used as a template for filtering the ChemBridge database based on shape fingerprints and 58 compound were identified that had >85% shape similarity. Docking was performed on these compounds. There were −1600 poses that were scored, and the best pose of the control compound placed 9^{th} with a score of −7.03 kcal/mol. There were 8 compounds above the control, with the highest having a score of −7.4 kcal/mol. As such, the control is ranked relatively high. We also examined the placement of these 8 compounds in the subpocket, in terms of the degree to which they superpose with the control compound as follows: (R26=good overlap; R31=good overlap; R32=good overlap; R29=partial overlap; R 30=partial overlap; R27=minimal overlap; R28=minimal overlap).

It will be readily apparent to one skilled in the art that varying substitutions and modifications may be made to the invention disclosed herein without departing from the scope and spirit of the invention. The invention illustratively described herein suitably may be practiced in the absence of any element or elements, limitation or limitations which is not specifically disclosed herein. The terms and expressions which have been employed are used as terms of description and not of limitation, and there is no intention in the use of such terms and expressions of excluding any equivalents of the features shown and described or portions thereof, but it is recognized that various modifications are possible within the scope of the invention. Thus, it should be understood that although the present invention has been illustrated by specific embodiments and optional features, modification and/or variation of the concepts herein disclosed may be resorted to by those skilled in the art, and that such modifications and variations are considered to be within the scope of this invention.

Citations to a number of patent and non-patent references are made herein. The cited references are incorporated by reference herein in their entireties. In the event that there is an inconsistency between a definition of a term in the specification as compared to a definition of the term in a cited reference, the term should be interpreted based on the definition in the specification.

1. A method for treating breast cancer in a subject in need thereof, wherein the breast cancer is associated with RAD52 biological activity and the breast cancer is selected from the group consisting of BRCA1-deficient breast cancer, BRCA2-deficient breast cancer, and PALB2-deficient cancer, and the method comprises administering a therapeutic agent that inhibits of RAD52 mediated annealing of ssDNA with an IC₅₀ of less than about 10 μM.

2.-5. (canceled)

6. The method of claim 1, wherein the therapeutic agent inhibits binding of RAD52 to ssDNA.

7. The method of claim 6, wherein the therapeutic agent inhibits binding of RAD52 to ssDNA with an IC₅₀ of less than about μM.

8.-11. (canceled)

12. The method of claim 1, wherein the ssDNA is RPA-coated ssDNA.

13. (canceled)

14. The method of claim 1, wherein the therapeutic agent is selected from the following compounds, hydrates thereof, or pharmaceutically acceptable salts thereof:

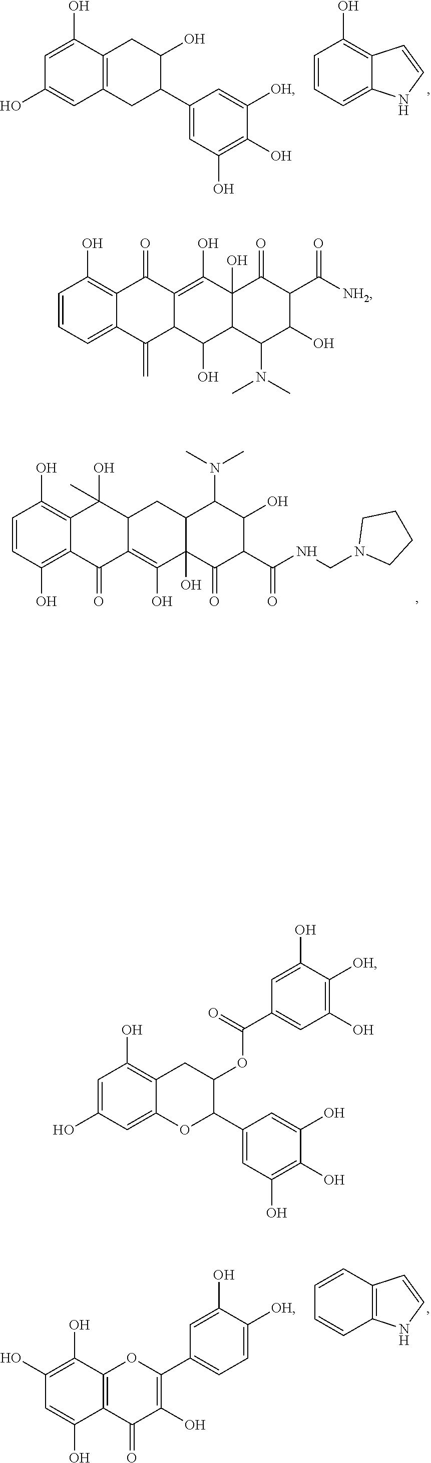

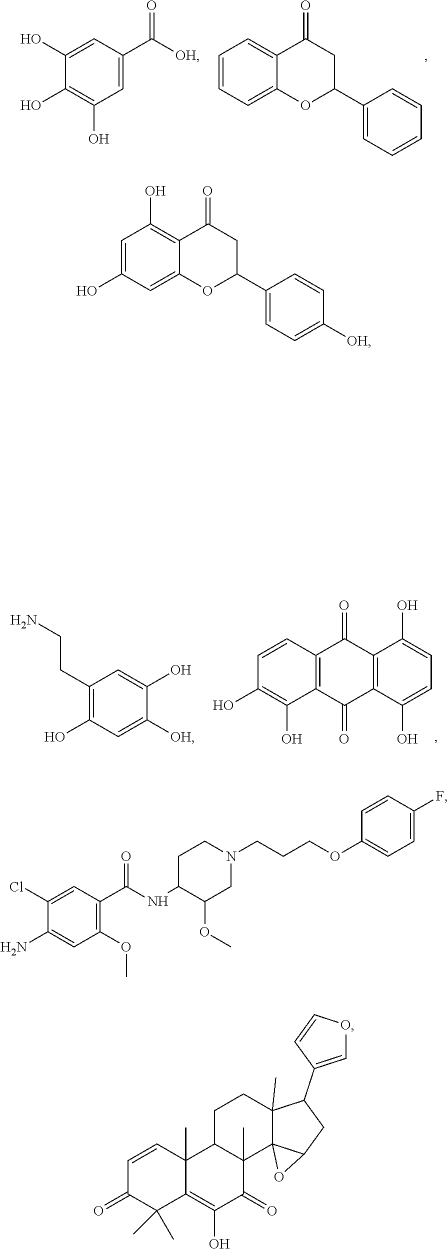

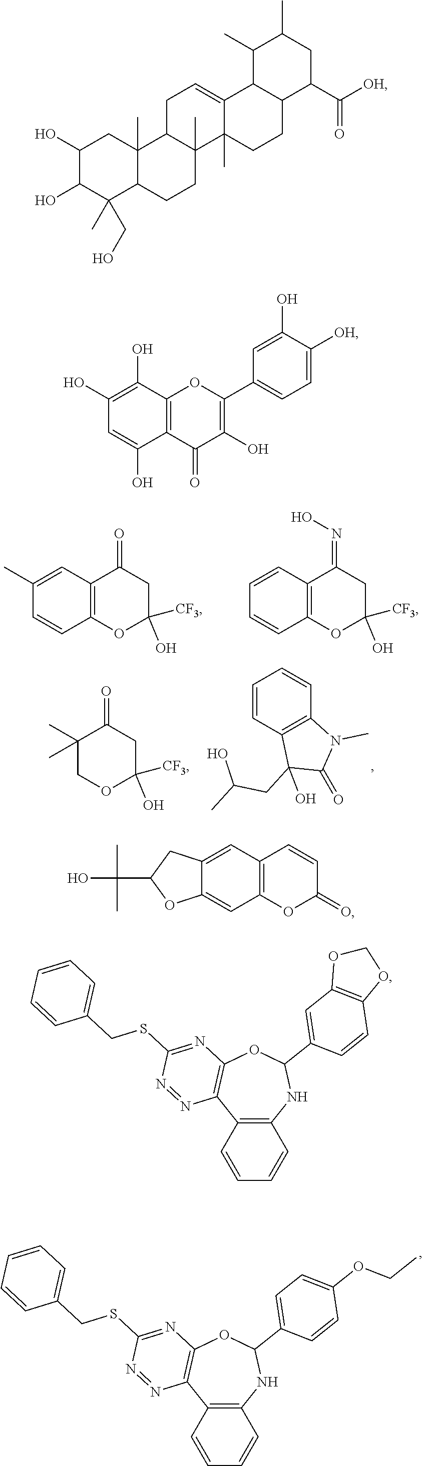

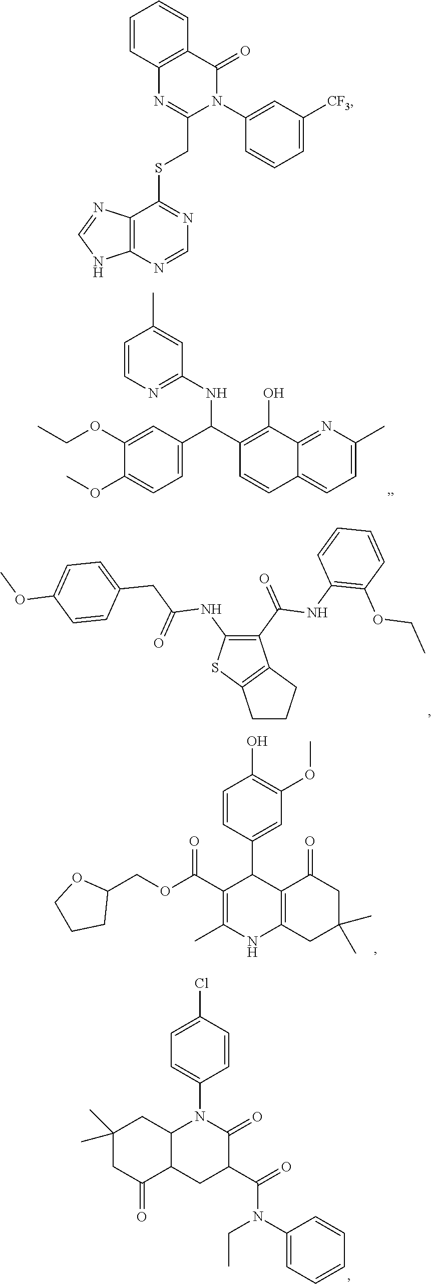

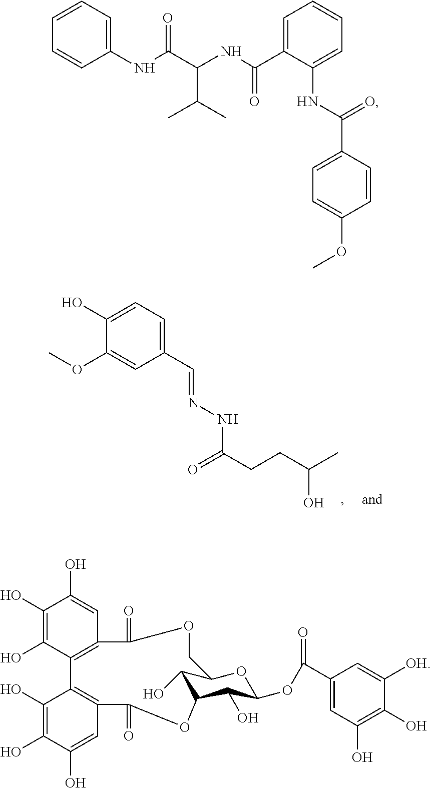 [SMILES: COc1ccc(C(=O)Nc2ccccc2C(=O)NC(C(=O)Nc2ccccc2)C(C)C)cc1 ; COc1cc(/C=N/NC(=O)CCC(C)O)ccc1O ; O=C(O[C@@H]1OC2COC(=O)c3cc(O)c(O)c(O)c3-c3c(cc(O)c(O)c3O)C(=O)O[C@H](C1O)[C@@H]2O)c1cc(O)c(O)c(O)c1]

15. The method of claim 1, wherein the compound has a structure: 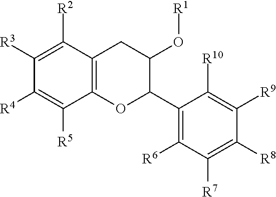
wherein R¹ is H, C1-C6 alkyl, C1-C6 alkoxy, or 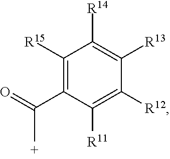
wherein R¹¹, R¹², R¹³, R¹⁴, and R¹⁵ are each independently selected from H, —OH, halo, C1-C6 alkyl, and C1-C6 alkoxy; and
wherein R², R³, R⁴, R⁵, R⁶, R⁷, R⁸, R⁹ and R¹⁰ are each independently selected from H, —OH, halo, C1-C6 alkyl, and C1-C6 alkoxy.

16. The method of claim 15 wherein the compound has a formula:

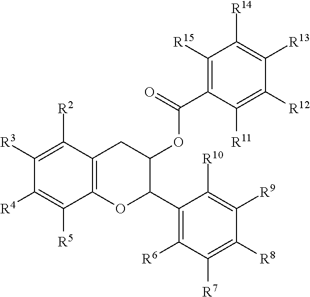

17. The method of claim 1, wherein the compound is selected from the group consisting of (−)-epigallocatechin, epigallocatechin-3-monogallate, naringenin, taxifolin, myricetin, tricetin, cyanidin, eriodictyol, 3-methylquercetin, robinetin, tamarixetin, 3′-O-methylepicatechin, meciadanol, theaflavine, 5,7,3′-trihydroxy-3,4′-dimethoxyflavone, 2H-1-benzopyran-3,7-dio1,2-(3,4-dihydroxyphenyl)-3,4-dihydro-, petunidin, 4′-methylepigallocatechin, delphinidin, (+)-Epicatechin, taxifolin, mearnsetin, Fisetin 3-methyl ether, 7,3′,4′,5′-Tetrahydroxyflavone, 3,5,7,4′-Tetrahydroxyflavan, fustin, leucocyanidin, melacacidin, cyrtominetin, (−)-Gallocatechin, 2H-1-Benzopyran-5,7-diol, 3,4-dihydro-2-(4-hydroxyphenyl)-, robinetinidol, 3′-O-methylcatechin, Epicatechin 3′,4′-dimethyl ether, leukoefdin, 2-(3,4-dihydroxyphenyl)-3,5,7-trihydroxy-2,3-dihydronaphthalene-1,4-dione, luteoforol, 7,4′-Dihydroxyflavan, luteoforol, leukoefdin, afzelechin, Fisetinidol, Apiforol, Dihydrokaempferide, leukoefdin, Laricitrin, 5,7,4′-Tri-O-methylcatechin, (?)-Epicatechin quione, 4H-1-Benzopyran-4-one, 5,7 ,8-trihydroxy-2-(3,4,5-trihydroxyphenyl)-, 3′-Hydroxy-4′-O-methylglabridin, Mesquitol, Tricetinidin, (+)-Epiaromadendrin, L-Epicatechin, 1,2-Benzenediol, 4-(3,4-dihydro-7-hydroxy-2H-1-benzopyran-2-yl)-, Pinomyricetin, Epidistenin, 4′-O-methyepicatechin, Hibiscetin, Epimesquitol-4beta-ol, 4H-1-Benzopyran-4-one, 5,7-dihydroxy-2-(4-hydroxyphenyl)-3-methyl-, 4H-1-Benzopyran-4-one, 5-hydroxy-2-(3,4,5-trihydroxyphenyl)-, 2,3-Dihydrogossypetin, 2-(4-hydroxyphenyl)-3,4-dihydro-2h-chromene-4,5,7-triol, epi-Catechol, (2S)-dihydrotricetin, Taxifolin 3-O-acetate, Arachidoside, Leuco-fisetinidin, 3,5,7-trihydroxy-2-(3,4,5-trihydroxyphenyl)-3,4-dihydro-2H-1-benzopyran-4-one, “Isoetin, Guibourtinidol, 4′-O-Methylcatechin, Epicatechin 5,3′-dimethyl ether, 3-O-Methylepicatechin, Keto-teracacidin, Apigeniflavan, and 3,5,8,3′,4′,5′-Hexahydroxyflavone.

18. A pharmaceutical composition comprising as a therapeutic agent a compound selected from the following compounds, hydrates thereof, or pharmaceutically acceptable salts thereof:

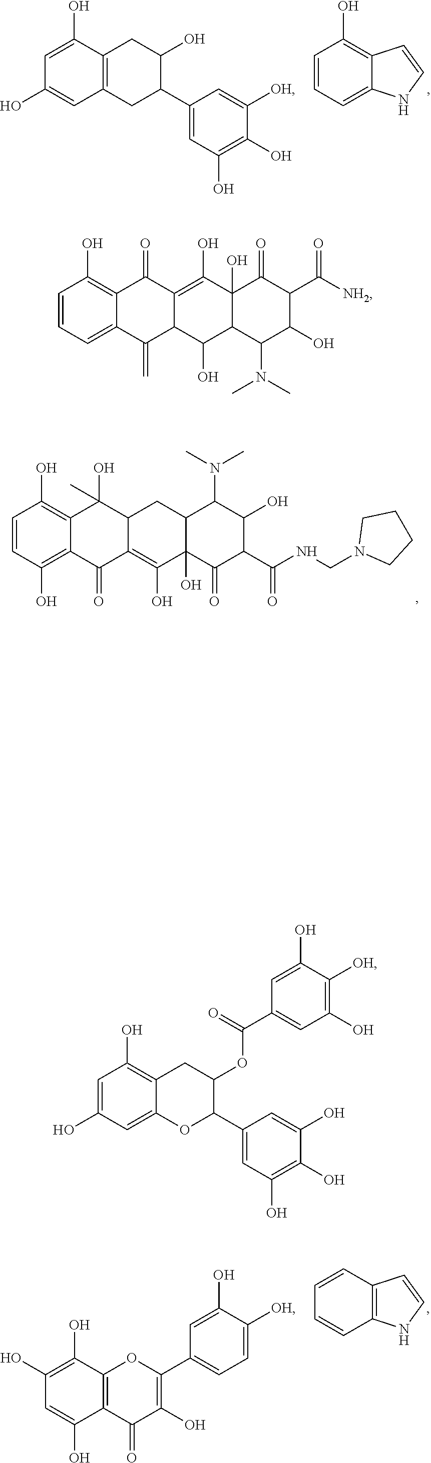

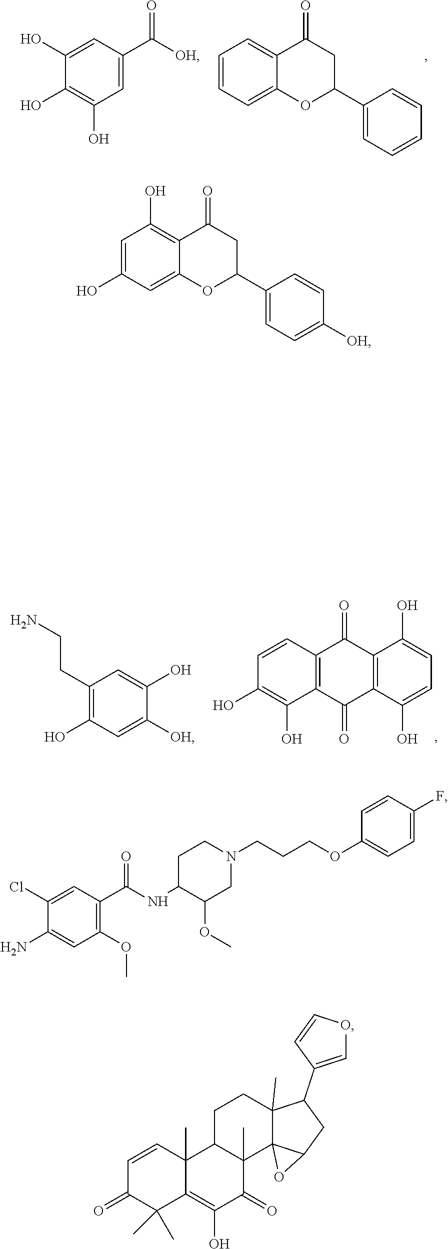

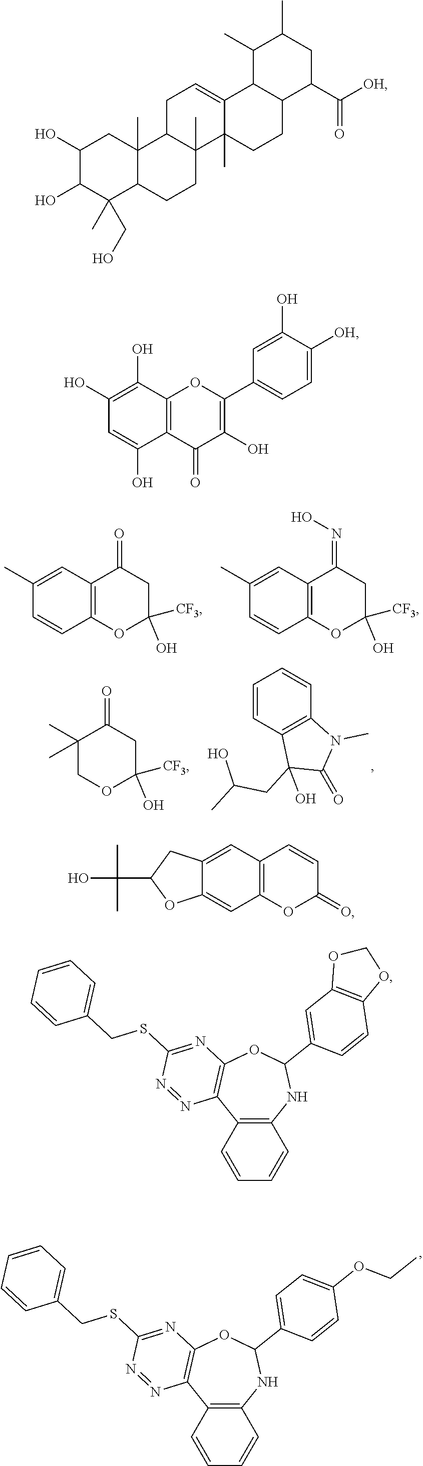

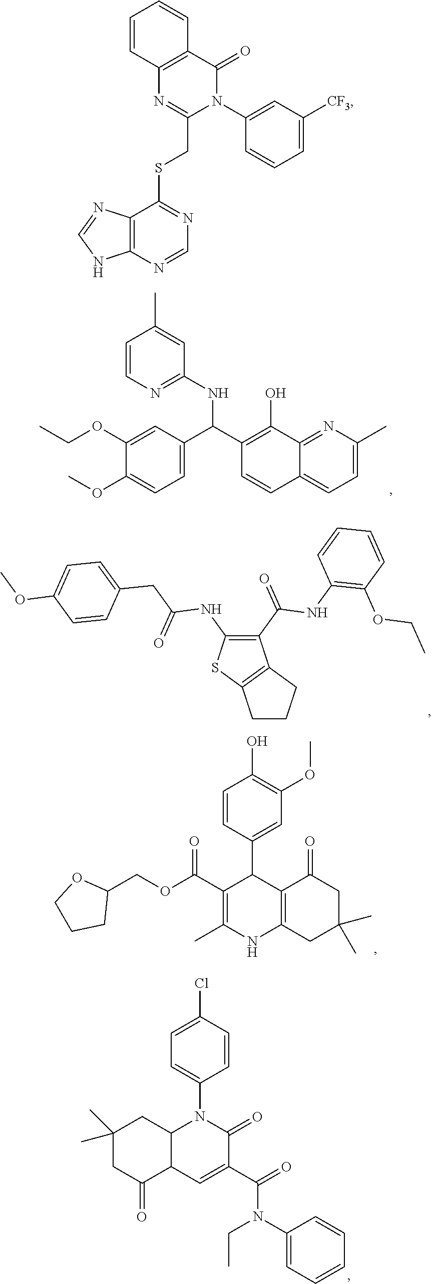

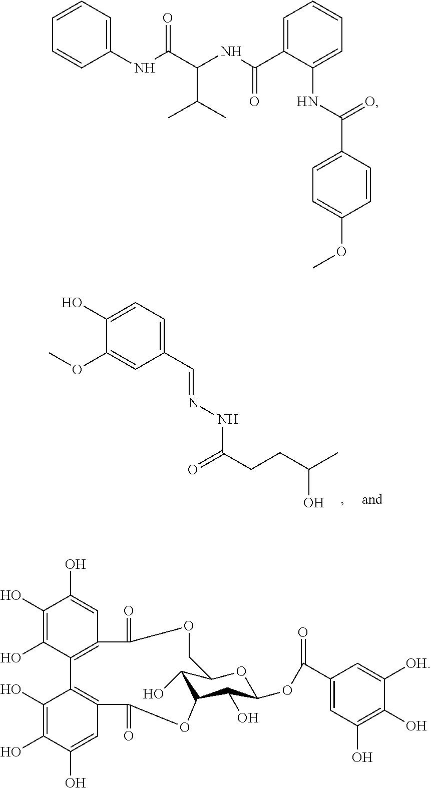 [SMILES: COc1ccc(C(=O)Nc2ccccc2C(=O)NC(C(=O)Nc2ccccc2)C(C)C)cc1 ; COc1cc(/C=N/NC(=O)CCC(C)O)ccc1O ; O=C(O[C@@H]1OC2COC(=O)c3cc(O)c(O)c(O)c3-c3c(cc(O)c(O)c3O)C(=O)O[C@H](C1O)[C@@H]2O)c1cc(O)c(O)c(O)c1]

19.-21. (canceled)

22. A method for identifying an inhibitor of RAD52 biological activity, the method comprising contacting RAD52 with a compound and determining if the compound binds RAD52 and/or inhibits binding of RAD52 to ssDNA and/or inhibits RAD52 annealing of ssDNA, thereby identifying the inhibitor of RAD52 biological activity.
